SEQUENCE LISTING (47 sequences)

SEQ ID NO: 1 - protein, 1368 aa, Artificial Sequence
  Synthetic Polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1368 aa, S. pyogenes [sequence omitted]

SEQ ID NO: 3 - DNA, 12 bp, Artificial Sequence
  Synthetic Polynucleotide
tgcgcaccctta

SEQ ID NO: 4 - DNA, 74 bp, Artificial Sequence
  Synthetic Polynucleotide
aattgtgagcggataacaattgacattgtgagcggataacaagatactgagcacatcagcaggacgcactgacc

SEQ ID NO: 5 - DNA, 74 bp, Artificial Sequence
  Synthetic Polynucleotide
tccctatcagtgatagagattgacatccctatcagtgatagagatactgagcacatcagcaggacgcactgacc

SEQ ID NO: 6 - DNA, 82 bp, Artificial Sequence
  Synthetic Polynucleotide
atgcgcacccttagcgagaggtttatcattaaggtcaacctctggatgttgtttcggcatcctgcattgaatctgagttact

SEQ ID NO: 7 - DNA, 131 bp, Artificial Sequence
  Synthetic Polynucleotide
gtcagaaaaaacgggtttcctgaattccaacatggatgctgatttatatgggtataaatgggcccgcgataatgtcgggcaatcaggtgcgacaatctatcggaattcaggaaaacagacagtaactcaga

SEQ ID NO: 8 - DNA, 134 bp, Artificial Sequence
  Synthetic Polynucleotide
gtcagaaaaaacgggtttcctgaattccagctaatttccgcgctcggcaagaaagatcatgccctcttgatcgattgccgctcactggggaccaaagcagtttccgaattcaggaaaacagacagtaactcaga

SEQ ID NO: 9 - DNA, 134 bp, Artificial Sequence
  Synthetic Polynucleotide
gtcagaaaaaacgggtttcctgaattcacccaacttaatcgccttgcagcacatccccctttcgccagctggcgtaatagcgaagaggcccgcaccgatcgccctgaattcaggaaaacagacagtaactcaga

SEQ ID NO: 10 - DNA, 30 bp, Artificial Sequence
  Synthetic Polynucleotide
ggttgatattgattcagaggtataaaacga

SEQ ID NO: 11 - DNA, 30 bp, Artificial Sequence; identical to SEQ ID NO: 10
  Synthetic Polynucleotide

SEQ ID NO: 12 - DNA, 81 bp, Artificial Sequence
  Synthetic Polynucleotide
gtcagaaaaaacgggtttcctgaattcgggtataaatgggcccgcgataatggaattcaggaaaacagacagtaactcaga

SEQ ID NO: 13 - DNA, 101 bp, Artificial Sequence
  Synthetic Polynucleotide
gtcagaaaaaacgggtttcctgaattctgatttatatgggtataaatgggcccgcgataatgtcgggcaatcgaattcaggaaaacagacagtaactcaga

SEQ ID NO: 14 - DNA, 121 bp, Artificial Sequence
  Synthetic Polynucleotide
gtcagaaaaaacgggtttcctgaattcacatggatgctgatttatatgggtataaatgggcccgcgataatgtcgggcaatcaggtgcgacagaattcaggaaaacagacagtaactcaga

SEQ ID NO: 15 - DNA, 131 bp, Artificial Sequence; identical to SEQ ID NO: 7
  Synthetic Polynucleotide

SEQ ID NO: 16 - DNA, 221 bp, Artificial Sequence
  Synthetic Polynucleotide
gtcagaaaaaacgggtttcctgaattcgagccatattcaacgggaaacgtcttgctcgaggccgcgattaaattccaacatggatgctgatttatatgggtataaatgggcccgcgataatgtcgggcaatcaggtgcgacaatctatcgattgtatgggaagcccgatgcgccagagttgtttctgaaacagaattcaggaaaacagacagtaactcaga

SEQ ID NO: 17 - DNA, 134 bp, Artificial Sequence
  Synthetic Polynucleotide
gtcagaaaaaacgggtttcctgaattcggtgatcttcttgccggcgattttttcaaccgagactcactaacaccagccgtcgatctcgtaggtttcgaggggcaggaattcaggaaaacagacagtaactcaga

SEQ ID NO: 18 - DNA, 134 bp, Artificial Sequence
  Synthetic Polynucleotide
gtcagaaaaaacgggtttcctgaattcctgcccctcgaaacctacgagatcgacggctggtgttagtgagtctcggttgaaaaaatcgccggcaagaagatcaccgaattcaggaaaacagacagtaactcaga

SEQ ID NO: 19 - DNA, 128 bp, Artificial Sequence
  Synthetic Polynucleotide
gtcagaaaaaacgggtttcctgaattcaccgcctgggccgagtgcggagatacgacgtttgtgcgtaatctcagacagcgggttgttctggtccataaagaattcaggaaaacagacagtaactcaga

SEQ ID NO: 20 - DNA, 128 bp, Artificial Sequence
  Synthetic Polynucleotide
gtcagaaaaaacgggtttcctgaattcgaatggctgcgccatgcggacgatcgtgtcatagaccgccgagtcaccatggggatggtatttaccgattacgaattcaggaaaacagacagtaactcaga

SEQ ID NO: 21 - DNA, 156 bp, Artificial Sequence
  Synthetic Polynucleotide
  misc_feature (63)..(68): n is a, c, g, or t
  misc_feature (87)..(92): n is a, c, g, or t
gtcagaaaaaacgggtttcctgaattcaatgtgagttagctcactcattaggcaccccaggcnnnnnnctttatgcttccggctcgnnnnnngtgtggaattgtgagcggataacaatttcacacaggaattcaggaaaacagacagtaactcaga

SEQ ID NO: 22 - DNA, 597 bp, Artificial Sequence
  Synthetic Polynucleotide
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 23 - DNA, 20 bp, Artificial Sequence
  Synthetic Polynucleotide
tgagcggcaatcgattcatt

SEQ ID NO: 24 - DNA, 20 bp, Artificial Sequence
  Synthetic Polynucleotide
tcacgcgaattatttaccgc

SEQ ID NO: 25 - DNA, 20 bp, Artificial Sequence
  Synthetic Polynucleotide
gcttaccgtcattcatcatt

SEQ ID NO: 26 - DNA, 17 bp, Artificial Sequence
  Synthetic Polynucleotide
  misc_feature (6)..(11): n is a, c, g, or t
atgccnnnnnnatcgat

SEQ ID NO: 27 - DNA, 30 bp, Artificial Sequence
  Synthetic Polynucleotide
  misc_feature (1)..(6): n is a, c, g, or t
  misc_feature (25)..(30): n is a, c, g, or t
nnnnnnctttatgcttccggctcgnnnnnn

SEQ ID NO: 28 - DNA, 1149 bp, Artificial Sequence
  Synthetic Polynucleotide
atgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgcctaatgaatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctga

SEQ ID NO: 29 - DNA, 136 bp, Artificial Sequence
  Synthetic Polynucleotide
  misc_feature (63)..(67): n is a, c, g, or t
gtcagaaaaaacgggtttcctgaattcgctaatttccgcgctcggcaagaaagatcatgcctnnnnnbtcgattgccgctcactggggaccaaagcagtttccatgcgaattcaggaaaacagacagtaactcaga

SEQ ID NO: 30 - DNA, 136 bp, Artificial Sequence
  Synthetic Polynucleotide
  misc_feature (65)..(69): n is a, c, g, or t
gtcagaaaaaacgggtttcctgaattcgttggcagcgaatacaacacccgtcgtgaacagtgcgnnnnnhgtgcgcgtttcttccagcagccagccctgcgtgatgtgaattcaggaaaacagacagtaactcaga

SEQ ID NO: 31 - DNA, 14 bp, Artificial Sequence
  Synthetic Polynucleotide
ggcgatctaacgcg

SEQ ID NO: 32 - DNA, 20 bp, Artificial Sequence
  Synthetic Polynucleotide
ggactgccgctcgctggcga

SEQ ID NO: 33 - DNA, 45 bp, Artificial Sequence
  Synthetic Polynucleotide
  misc_feature (21)..(25): n is a, c, g, or t
acacgacgctcttccgatctnnnnnctggaaagcgggcagtgagc

SEQ ID NO: 34 - DNA, 61 bp, Artificial Sequence
  Synthetic Polynucleotide
  misc_feature (32)..(36): n is a, c, g, or t
cggcattcctgctgaaccgctcttccgatctnnnnncccagtcacgacgttgtaaaacgac

SEQ ID NO: 35 - DNA, 54 bp, Artificial Sequence
  Synthetic Polynucleotide
  misc_feature (21)..(25): n is a, c, g, or t
acacgacgctcttccgatctnnnnngtttgtaggctgtaactgcgggatcatgg

SEQ ID NO: 36 - DNA, 56 bp, Artificial Sequence
  Synthetic Polynucleotide
  misc_feature (32)..(36): n is a, c, g, or t
cggcattcctgctgaaccgctcttccgatctnnnnntcacgcagggctggctgctg

SEQ ID NO: 37 - DNA, 48 bp, Artificial Sequence
  Synthetic Polynucleotide
  misc_feature (21)..(25): n is a, c, g, or t
acacgacgctcttccgatctnnnnngctcggcaagaaagatcatgcca

SEQ ID NO: 38 - DNA, 56 bp, Artificial Sequence
  Synthetic Polynucleotide
  misc_feature (32)..(36): n is a, c, g, or t
cggcattcctgctgaaccgctcttccgatctnnnnnctgctggaagaaacgcgcag

SEQ ID NO: 39 - DNA, 18 bp, Artificial Sequence
  Synthetic Polynucleotide
  misc_feature (7)..(12): n is a, c, g, or t
atgcctnnnnnntcgatt

SEQ ID NO: 40 - DNA, 18 bp, Artificial Sequence
  Synthetic Polynucleotide
  misc_feature (7)..(12): n is a, c, g, or t
agtgcgnnnnnngtgcgc

SEQ ID NO: 41 - DNA, 17 bp, Artificial Sequence
  Synthetic Polynucleotide
atgccctcttgatcgat

SEQ ID NO: 42 - DNA, 17 bp, Artificial Sequence
  Synthetic Polynucleotide
atgcctaatgaatcgat

SEQ ID NO: 43 - DNA, 18 bp, Artificial Sequence
  Synthetic Polynucleotide
catgcctaatgaatcgat

SEQ ID NO: 44 - DNA, 18 bp, Artificial Sequence
  Synthetic Polynucleotide
catgcccaattgatcgat

SEQ ID NO: 45 - DNA, 18 bp, Artificial Sequence
  Synthetic Polynucleotide
catgccctcttgatcgat

SEQ ID NO: 46 - DNA, 174 bp, Artificial Sequence
  Synthetic Polynucleotide
aagaaagatcatgcctaatgaatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcag

SEQ ID NO: 47 - DNA, 174 bp, Artificial Sequence
  Synthetic Polynucleotide
aataaagatcatgccacgtacttcgattgccgctcactggggaccaaagcagtttccatgcccaaaggtgtggctgtcgtcatcatcaacagtaacttcaaacgtaccctggttggcagcgaatacaacacccgtcgtgaacagtgcgagttatctgcgcgtttcttccagcag

(19) United States
(12) Patent Application Publication
(10) Pub. No.: US 2018/0127759 A1
(43) Pub. Date: May 10, 2018

(54) DYNAMIC GENOME ENGINEERING

(71) Applicant: Massachusetts Institute of Technology, Cambridge, MA (US)

(72) Inventors: Timothy Kuan-Ta Lu, Cambridge, MA (US); Fahim Farzadfard, Boston, MA (US)

(21) Appl. No.: 15/796,551

(22) Filed: Oct. 27, 2017

Related U.S. Application Data
(60) Provisional application No. 62/442,788, filed on Jan. 5, 2017.
(60) Provisional application No. 62/421,839, filed on Nov. 14, 2016.
(60) Provisional application No. 62/414,633, filed on Oct. 28, 2016.

(51) Int. Cl. C12N 15/113; C12N 9/22; C12N 15/70; C12N 9/12; C12Q 1/6876; C12N 15/74; C07K 16/00; C12N 15/90

(52) U.S. Cl. CPC C12N 15/1137; C12N 9/22; C12N 15/70; C12N 9/1276; C12Y 207/07049; C07K 2317/14; C12N 15/74; C07K 16/00; C12N 15/902; C12N 2310/20; C12Q 1/6876

(57) ABSTRACT

Provided herein, in some embodiments, are genomic editing constructs that can achieve nearly 100% recombination efficiency within a select population of bacterial cells.

### RELATED APPLICATIONS

This application claims the benefit under 35 U.S.C. § 119(e) of U.S. provisional application No. 62/442,788, filed Jan. 5, 2017, U.S. provisional application No. 62/421,839, filed Nov. 14, 2016 and U.S. provisional application No. 62/414,633, filed Oct. 28, 2016, each of which is incorporated by reference herein in its entirety.

### GOVERNMENT SUPPORT

This invention was made with Government support under Grant No. N00014-13-1-0424 awarded by the Office of Naval Research and under Grant Nos. OD008435 and P50 GM098792 awarded by the National Institutes of Health. The Government has certain rights in the invention.

### BACKGROUND

Genomic DNA is an evolvable functional memory that records history of adaptive changes over evolutionary time-scales. Evolution is a continuous process of genetic diversification and phenotypic selection that tunes genetic makeup of living organisms and maximizes their fitness in a given environment over evolutionary timescales. Although genetic variation is the driving force of evolution, elevating mutation rate globally is a highly inefficient strategy to optimize the fitness of the cells, as infrequent beneficial mutations are often masked by much more frequent deleterious ones. As the size of mutable genetic materials increases, the likelihood of occurrence of deleterious mutations over beneficial mutations also increases. For example, the mutation rate (per nucleotide base pair) of asexually reproducing organisms (e.g., prokaryotes) is negatively correlated with an organism's genome size. Changing environments pose a challenge to living organisms. The ability to selectively increase diversity in specific regions of a genome, and to adjust such response in response to certain cues, enables an organism to tune its ability to evolve and adapt in uncertain environments.

### SUMMARY

The gene editing systems and nucleic acid constructs (‘gene editing constructs’) of the present disclosure enable high-efficiency, precise, autonomous and dynamic genomic editing/writing of select bacterial genomes within a larger bacterial community, for example. Unexpectedly, this high-efficiency gene editing technology, which is based in part on synthetic oligonucleotide recombineering principles, may be implemented in bacterial cells having a fully active mismatch repair (MMR) system. Additionally, this system can achieve a selective increase of more than eight orders of magnitude in the rate of incorporation of pre-defined mutations into specific genomic regions over the background mutation rate. The gene editing constructs of the present disclosure integrate certain elements from the SCRIBE genomic editing systems (Farzadfard, T. K. Lu, Science 346, 1256272 (2014), incorporated herein by reference) and the CRISPR genomic editing systems (Jinek et al., Science 337, 6096, 816-821 (2012), incorporated herein by reference) to provide tools that can achieve nearly 100% recombination efficiency within a select population of bacterial cells, while avoiding lethal double-strand breaks in genomic DNA. Unlike current gene editing strategies, the gene editing system of the present disclosure does not require cis-encoded sequence on the target and, thus, the entire genome (any loci within the genome) may be used for high-efficiency editing and memory applications. Further, unlike gene editing strategies that rely on counterselection by CRISPR-Cas9 nucleases, the gene editing system of the present disclosure, in some embodiments, does not require the presence of a PAM sequence on the target, thereby enabling multiple rounds of allele replacement on the same target.

Experimental data presented herein show (1) that this gene editing system can be transcriptionally controlled, thus enabling computation and memory applications; (2) that the system can be delivered into cells via various delivery mechanisms, including transduction and conjugation, enabling efficient and specific genome writing in bacteria within bacterial communities; and (3) that high-efficiency gene editing can be used to record transient spatial information into genomic DNA, allowing the reduction of multidimensional interactomes into a one-dimensional DNA sequence space, thus facilitating the study of complex cellular interactions. Additionally, when combined with a continuous delivery system, this high-efficiency gene editing platform enables the continuous optimization of a trait of interest when coupled to appropriate selections or screens. This system can also be used to selectively increase the de novo mutation rate of desired genomic loci while minimizing the background mutation rate, as opposed to using a generalized hypermutator phenotype, thus allowing one to tune the evolvability of specific genomic segments. Thus, the high-efficiency gene editing (writing) system as provided herein enables unprecedented genomic editing, cellular memory, connectome mapping, and targeted evolution applications.

Provided herein, in some embodiments, is an engineered nucleic acid construct comprising: (a) a nucleotide sequence encoding a guide RNA targeting an exonuclease; (b) a nucleotide sequence encoding a single-stranded msrRNA and a single-stranded msdDNA modified to contain a targeting sequence, wherein (b) is flanked by a pair of inverted repeat sequences; and (c) a one nucleotide sequence encoding a reverse transcriptase protein.

Also provided herein are compositions and kits comprising the engineered nucleic acid constructs (gene editing constructs) of the present disclosure.

A cell may comprise, for example, (a) an engineered nucleic acid construct, (b) a single-stranded DNA-annealing recombinase protein, and (c) a catalytically-inactive Cas9 protein.

In some embodiments, a cell comprises (a) an engineered nucleic acid encoding a guide RNA targeting an exonuclease, and (b) an engineered nucleic acid comprising (i) a nucleotide sequence encoding a single-stranded msrRNA and a single-stranded msdDNA modified to contain a targeting sequence, wherein (b) is flanked by a pair of inverted repeat sequences, and (ii) a nucleotide sequence encoding a reverse transcriptase protein.

In some embodiments, a cell comprises (a) an engineered nucleic acid encoding a guide RNA targeting an exonuclease, (b) an engineered nucleic acid encoding a single-stranded msrRNA and a single-stranded msdDNA modified to contain a targeting sequence, wherein (b) is flanked by a pair of inverted repeat sequences, and (c) an engineered nucleic acid encoding a reverse transcriptase protein.

A cell may further comprise, in some embodiments, an engineered nucleic acid encoding a single-stranded DNA-annealing recombinase protein. In some embodiments, a cell further comprises an engineered nucleic acid encoding a catalytically-inactive Cas9 protein.

Also provided herein are methods comprising delivering to a cell an engineered nucleic acid construct of the present disclosure, wherein the cell comprises at least one target nucleotide sequence that is complementary to the targeting sequence of the single-stranded msdDNA.

In some embodiments, a method comprises delivering to a cell (a) an engineered nucleic acid constructs of the present disclosure, (b) a single-stranded DNA-annealing recombinase protein, and (c) a catalytically-inactive Cas9 protein.

In some embodiments, a method comprises delivering to a cell (a) an engineered nucleic acid encoding a guide RNA targeting an exonuclease, and (b) an engineered nucleic acid comprising (i) a nucleotide sequence encoding a single-stranded msrRNA and a single-stranded msdDNA modified to contain a targeting sequence, wherein (b) is flanked by a pair of inverted repeat sequences, and (ii) a nucleotide sequence encoding a reverse transcriptase protein.

In some embodiments, a method comprises delivering to a cell (a) an engineered nucleic acid encoding a guide RNA targeting an exonuclease, (b) an engineered nucleic acid encoding a single-stranded msrRNA and a single-stranded msdDNA modified to contain a targeting sequence, wherein (b) is flanked by a pair of inverted repeat sequences, and (c) an engineered nucleic acid encoding a reverse transcriptase protein.

Also provided herein are methods of modifying a bacterial cell subpopulation, comprising delivering to at least one bacterial cell of the subpopulation an engineered nucleic acid construct comprising (a) a nucleotide sequence encoding a guide RNA targeting an exonuclease, and (b) a nucleotide sequence encoding a single-stranded msrRNA and a single-stranded msdDNA modified to contain a targeting sequence that targets a gene specific to the bacterial cell subpopulation, wherein (b) is flanked by a pair of inverted repeat sequences, and (c) a nucleotide sequence encoding a reverse transcriptase protein.

Further provided herein are methods of activating a naturally silent gene in a bacterial cell, comprising delivering into the bacteria cell an engineered nucleic acid construct comprising (a) a nucleotide sequence encoding a guide RNA targeting an exonuclease, and (b) a nucleotide sequence encoding a single-stranded msrRNA and a single-stranded msdDNA modified to contain a targeting sequence that targets a naturally silent gene in a bacterial cell, wherein (b) is flanked by a pair of inverted repeat sequences, and (c) a nucleotide sequence encoding a reverse transcriptase protein.

Some embodiments provide methods of diversifying a genomic locus in a cell, comprising delivering to the cell an engineered nucleic acid construct comprising (a) a nucleotide sequence encoding a guide RNA targeting an exonuclease, (b) a nucleotide sequence encoding a single-stranded msrRNA and a single-stranded msdDNA modified to contain a targeting sequence that targets a genomic locus in a cell, and (c) a nucleotide sequence encoding an error-prone reverse transcriptase protein, wherein (b) is flanked by a pair of inverted repeat sequences.

Other embodiments provide methods of mapping cellular interactions, comprising (a) delivering to a donor cell within a population of recipient cells a transfer vector comprising a gene editing system that introduces a genetic barcode into a locus of the genome of the donor cells and a locus of the genome of the recipient cells, (b) collecting the donor cell and at least one recipient cell, and (c) sequencing the locus of the genome of the donor cells and the locus of the genome of the at least one recipient cell to map interactions among the donor cell and the at least one recipient cell.

Gene editing systems used in methods of mapping cellular interactions may comprise, for example, (a) a nucleotide sequence encoding a guide RNA targeting an exonuclease, (b) a nucleotide sequence encoding a single-stranded msrRNA and a single-stranded msdDNA modified to contain a targeting sequence that targets in a bacterial cell a nucleotide sequence encoding an antibody, wherein (b) is flanked by a pair of inverted repeat sequences, and (c) a nucleotide sequence encoding an error-prone reverse transcriptase protein.

Methods of improving fitness of bacterial cells are also provided. For example, such methods may include (a) delivering to bacterial cells an engineered nucleic acid construct comprising (i) a nucleotide sequence encoding a guide RNA targeting an exonuclease, (ii) a nucleotide sequence encoding a single-stranded msrRNA and a single-stranded msdDNA modified to contain a targeting sequence that targets an allele of a bacterial cell gene that adversely effects fitness of the bacterial cell under a stress condition, and (iii) a nucleotide sequence encoding an error-prone reverse transcriptase protein, wherein (ii) is flanked by a pair of inverted repeat sequences, (b) culturing bacterial cells of (a) under a stress condition; and (c) collecting viable bacterial cells of (b).

Also provided herein are bacterial cells that displays surface antibodies, comprising an engineered nucleic acid construct comprising (a) a nucleotide sequence encoding a guide RNA targeting an exonuclease, (b) a nucleotide sequence encoding a single-stranded msrRNA and a single-stranded msdDNA modified to contain a targeting sequence that targets in a bacterial cell a nucleotide sequence encoding an antibody, wherein (b) is flanked by a pair of inverted repeat sequences, and (c) a nucleotide sequence encoding an error-prone reverse transcriptase protein.

### BRIEF DESCRIPTION OF THE DRAWINGS

FIGS. 1A-1E: Genome editing system efficiency. (FIG. 1A) SCRIBE DNA writing efficiency in different knockout backgrounds determined by KanR reversion assay. (FIG. 1B) Model for retron-mediated recombineering. Intracellular recombinogenic oligonucleotides are generated likely due to degradation of template plasmid as well as msdDNA. ssDNA specific cellular exonucleases (XonA and RecJ) can further process these oligonucleotides into smaller non-recombinogenic (oligo)nucleotides. Alternatively, beta protein can bind to, protect and recombine these oligonucleotides into their genomic target loci. (FIG. 1C) Using CRISPRi to knockout cellular exonucleases for high efficiency genome editing using a genome editing system of the present disclosure. (FIG. 1D) High-efficiency genome editing for a screenable phenotype (galK reversion assay). galK_{OFF} reporter cells (white) were transformed with SCRIBE(galK)_{ON} plasmid, outgrown for 1 hour in LB and plated on MacConkey+Gal+antibiotic plates. The number of galK positive cells (pink) per transformants was used as a measure of recombinant frequency (FIG. 1E) Combining SCRIBE DNA writing with CRISPR nuclease to counter select against undesired (wild-type) alleles to increase the rate of enrichment of desired alleles within the population. gRNA against the galK_{OFF} locus under was placed under the control of aTc-inducible promoter and cloned into the SCRIBE(galK)_{ON} Plasmid. This plasmid was transformed into galK_{OFF} reporter strain harboring aTc-inducible Cas9 or dCas9 (as negative control) plasmids. After transformation, cells were outgrown for one hour, and plated on appropriate antibiotic plates. Single colonies from these plates were picked after 24 hours (˜30 generations), diluted to ˜10⁶ cells/ml in LB+Carb+Cm at presence or absence of aTc and grown for 12 hours up to saturation (˜10 generations). The allele frequency was determined by PCR amplification of the galK locus followed by high-throughput sequencing by MiSeq.

FIGS. 2A-2K: High-efficiency genome editing in MG1655 E. coli by delivering SCRIBE plasmid via different delivery methods and genome editing of bacteria via synthetic bacterial communities. (FIG. 2A) The SCRIBE(galK)_{ON}, dCas9, guide RNAs targeting recJ and xonA (collectively referred to as χSCRIBE(galK)_{ON}) were placed in a synthetic operon and cloned into a ColE1 plasmid encoding both M13 origin and RP4 origin of transfer. The plasmid was delivered into MG1655 galK_{OFF} reporter strain via different delivery method (chemical transformation, transduction and conjugation). For the transduction experiment, F plasmid was conjugated into the reporter strain from CJ236 strain. The gRNAs are flanked by Hammerhead Ribozyme (HHR) and hepatitis delta virus Ribozyme (HDVR) to allow in vivo processing and release of these gRNAs from the synthetic operon transcript. (FIG. 2B) Allele frequency within single colonies transformed with SCRIBE plasmids were determined by colony PCR of galK locus from transformants (24 hours after transformation) followed by Illumina sequencing. (FIG. 2C) Genome editing within a bacterial community via delivery of SCRIBE by transduction. Spontaneous Streptomycin mutants of the reporter strain (MG1655 St^{R} galK_{OFF}) were mixed (1:1) and co-cultured with an undefined bacterial culture obtained from mouse stool. Phagemid particles were added at MOI=50. The recombinant frequency was calculated as the number of pink colonies obtained on MacConkey+gal+St+Carb plates. (FIG. 2D) Genome editing within a bacterial community via delivery of SCRIBE by conjugation. MFDpir strains harboring SCRIBE(galK)_{ON} or SCRIBE(NS) plasmids were used as donor strains. The synthetic bacterial community described in FIG. 2C was used as the recipient culture. The donor and recipient cells were mixed with 100:1 ratio. The recombination efficiency was calculated as the number of pink colonies obtained on MacConkey+gal+St+Carb plates. (FIG. 2E) A schematic representation of a genetic circuit used to assess writing efficiency (left panel) as well as a schematic representation of enrichment of mutant alleles within a single transformant colony (right panel). (FIG. 2F) MG1655 exo− galKOFF reporter cells were transformed with the δHiSCRIBE(galK)_{ON} plasmid and population-wide recombinant frequency was measured by the galK reversion assay. The frequencies of galK_{ON} and galK_{OFF} alleles in individual transformant colonies obtained on LB plates were assessed one and two days after transformation using Sanger sequencing (FIG. 2G) as well as high-throughput Illumina sequencing (FIG. 2H). The sequences in FIG. 2G, from top to bottom, correspond to SEQ ID NOs: 43-45. (FIG. 2I) Allele frequencies of individual transformant colonies obtained on LB with appropriate selection were measured 24 hours after transformation by Illumina sequencing. (FIG. 2J) A conjugative χHiSCRIBE plasmid (harboring RP4 origin of transfer) was used to edit the MG1655 galKOFF StrR reporter strain in clonal population as well as within a synthetic bacterial community. (FIG. 2K) Efficiency of delivery of χHiSCRIBE plasmid by transduction and conjugation. To assess transduction efficiency of χHiSCRIBE phagemids, transduction mixtures were serially diluted and plated on LB+Str and LB+Str+Carb plates to measure the number of viable target cells and transductants, respectively. The ratio between the transductants and viable target cells was reported as transduction efficiency. To measure the conjugation efficiency of delivering the χHiSCRIBE plasmids, conjugation mixtures were serially diluted and plated on LB+Str and LB+Str+Carb plates, to measure the number of viable target cells and transconjugates, respectively. The ratio between the transconjugants and recipient cells was reported as conjugation efficiency.

FIGS. 3A-3E: Continuous evolution of a desired genomic locus via high efficiency SCRIBE (also referred to herein as HiSCRIBE). (FIG. 3A) diversity generation enabled by HiSCRIBE can be coupled to continuous selection to accelerate the rate of evolution of desired target sites. A randomized δHiSCRIBE (HiSCRIBE in a nuclease knockout background) library was encoded on phagemids that were continuously delivered into cells. In the presence of a selective pressure, δHiSCRIBE-mediated mutations lead to adaptive genetic changes that increase fitness. An increase in fitness results in faster replication and amplification of the associated genotype, increasing the chance that cells containing the genotype can undergo additional rounds of diversification. (FIG. 3B) The sequences of −35 and −10 boxes of the wild-type _{Plac} (Plac(WT)) and mutated P_{lac} (P_{lac}(mut)) targeted by a phagemid-encoded randomized δHiSCRIBE(P_{lac})_{rand} library in the evolution experiment. (FIG. 3C) Schematic representation of the evolution experiment. The −35 and −10 boxes of the Plac locus were targeted with an ssDNA library produced in vivo from a δHiSCRIBE phagemid library delivered by phagemid transduction. Cells that acquired beneficial mutations in their P_{lac} locus were expected to metabolize lactose better (indicated by darker gray shading) and be enriched in the population over time. (FIG. 3D) Growth rate profiles of cell populations exposed to δHiSCRIBE(P_{lac})rand and δHiSCRIBE(NS) (top) as well as the dynamics of P_{lac} alleles over the course of the experiment are shown as time series for cells exposed δHiSCRIBE(P_{lac})_{rand} phagemid library (middle). The bottom panel shows the identities of the most frequent alleles at the end of the experiment as well as the fold-change in β-galactosidase activity of those alleles in comparison to the WT and parental alleles. Alleles that are likely ancestors/descendants are linked by brackets. (FIG. 3E) The left panel shows the diversity of P_{lac} alleles observed as well as two additional parallel cultures, reported as the number of unique variants per sequencing read. The diversity of the P_{lac} locus in cultures exposed to the HiSCRIBE(P_{lac})rand phagemid library was significantly higher than those exposed to δHiSCRIBE(NS) phagemids. The right panel shows the dynamics of P_{lac} alleles for cultures that were exposed to δHiSCRIBE(NS) phagemids. The dynamics of allele enrichment for cells exposed to δHiSCRIBE(NS) and additional parallel evolution experiments are presented in FIGS. 9A and 9B.

FIGS. 4A-4E: De novo targeted mutagenesis via HiSCRIBE. (FIG. 4A) Instead of encoding a library of predefined mutations into HiSCRIBE, de novo mutations were introduced into HiSCRIBE-expressed ssDNAs during transcription and reverse transcription, since these processes are more error-prone than replication. Incorporation of these mutated ssDNAs into target loci results in targeted de novo diversity generation. To enhance the rate of ssDNA mutagenesis, AID was coexpressed with δHiSCRIBE. AID can deaminate cytidine in intracellularly expressed ssDNAs as well as ssDNA regions exposed during passage of replication forks, thus modulating mutation frequency and spectra. The δHiSCRIBE_AID operon was constructed by placing the AID gene into the δHiSCRIBE operon. Observed frequencies of RifR and NalR mutants were used to estimate locus-specific mutation rates of strains expressing different δHiSCRIBE plasmids at rpoB and gyrA loci, respectively, using the Maximum Likelihood Estimator (MSS-MLE) method. Error bars indicate 95% confidence intervals for each sample calculated based on 24 parallel cultures. Significant differences in mutation rates (p<0.01) are marked by asterisks. (FIG. 4B) Frequency of mutations observed in different positions along the rpoB locus. The light grey columns indicate on-target mutations (i.e., mutations that occurred within δHiSCRIBE(rpoB)_{WT} target site). Mutations in dC/dG positions are marked by plus signs. Fifty colonies were sequenced for each sample. (FIG. 4C) Mutation rates of rpoB and gyrA loci, estimated using MSS-MLE, in strains expressing the δHiSCRIBE_AID(rpoB)_{WT} plasmid and the aTc-inducible CRISPRi plasmid targeting E. coli Uracil-DNA glycosylase (ung). Error bars indicate 95% confidence intervals for each sample calculated based on 18 parallel cultures. Significant differences in mutation rates (p<0.01) are marked by asterisks. (FIG. 4D) Frequencies of RifR and NalR mutants, which harbor mutations in the rpoB and gyrA, respectively, observed in MG1655 ΔrecJ ΔxonA expressing different δHiSCRIBE plasmids. Bars indicate median and interquartile of each sample set. For each strain, the mutant frequencies in 24 parallel cultures were measured and the data was used to calculate the mutation rates shown in FIG. 4A. (FIG. 4E) Frequency of mutations at dC/dG positions based on the data in shown in FIG. 4B. AID expression increases the total frequency of mutations at dC/dG positions. However, in cells expressing δHiSCRIBE_AID(NS), dC/dG mutations mostly occur outside of the target sites. Expression of δHiSCRIBE_AID(rpoB)WT directs dC/dG mutations towards the target site (rpoB) and increases the frequency of on-target:total dC/dG mutations.

FIG. 5: Altering SCRIBE efficiency by modifying its expression level. DH5α PRO ΔrecJ ΔxonA kanR_{OFF} reporter cells were transformed with the constructs shown above and the recombinant efficiency was measured using kanR reversion assay. Using SCRIBE with a strong RBS upstream of beta resulted in highest recombinant frequency (˜36%).

FIG. 6: ssDNA homology length on SCRIBE DNA editing efficiency. Different KanR_{ON} ssDNA with different lengths of homology to the KanR_{OFF} target were tested by the KanR reversion assay. The efficiency of genome editing increases as the length of homology increase up to 35 bp homology. Larger homology size results in decrease in the editing efficiency, likely due to excessive secondary structures that could prevent efficient recombination, or alternatively inefficient ssDNA production by the retron system.

FIG. 7: Multiplexed writing in different loci using SCRIBE. A galK_{OFF} lacZ_{OFF} reporter strain was transduced with SCRIBE(galK)_{ON} or SCRIBE(lacZ)_{ON} (MOI=50) or both (MOI=100 each). Dilutions of the samples were spotted on LB+X-gal+IPTG+Carb or MacConkey+Gal+Carb plates to measure the frequency of recombinants in the lacZ locus (blue colonies) and galK locus (pink colonies), respectively.

FIG. 8: Genome editing in Pseudomonas putida. Two premature stop codons were introduced into the uracil phosphoribosyltransferase (Upp) ORF of P. putida using SCRIBE(Upp)_{OFF} targeting either the lagging strand or the leading strand of Upp ORF. While targeting the leading strand did not result in a significant increase in the editing efficiency, targeting the lagging strand promotes editing efficiency, demonstrating that SCRIBE is functional in other organisms. Knocking down the homologs of recJ and xonA in P. putida using CRISPRi result in a higher efficiency of editing, demonstrating that these exonucleases limit the efficiency of gene editing by SCRIBE in P. putida as well.

FIGS. 9A and 9B: Dynamics of P_{lac} alleles in the P_{lac} evolution experiment. Changes in P_{lac} alleles frequencies over the course of the experiment shown as time series for cells exposed to the δHiSCRIBE(NS) (top) or the δHiSCRIBE(P_{lac})_{rand} library phagemid particles (middle) for two additional parallel cultures of the experiment shown in FIG. 3. The identities of the most frequent alleles at the end of the experiment, as well as fold-change in β-galactosidase activity of the corresponding allele compared to the WT and parental alleles, are shown in the bottom tables. Alleles that are likely ancestors/descendants are linked by brackets. (FIG. 9A) Phagemid library #2. (FIG. 9B) Phagemid library #3.

FIG. 10: The E. coli genome contains 4 different ssDNA specific nucleases: recJ, xonA, exoVII (composed of two subunits encoded by xseA and xseB), and exoX. SCRIBE efficiency was improved by knocking out cellular exonucleases. The efficiency of SCRIBE was measured in different backgrounds using a kanR reversion assay. Knocking out exoX in the ΔrecJ ΔxonA background (which has been previously shown to result in improved efficiency), slightly increased the efficiency of SCRIBE. However, the viability of the triple nuclease mutant cells in the presence of SCRIBE cassette was significantly affected (a drop of approximately 2 logs in CFU count per ml in saturated cultures was observed), suggesting these cells are under a great stress. To avoid this selective pressure and possibility of occurrence of unwanted mutation, the double nuclease mutant (ΔrecJ ΔxonA) was chosen for the experiments. In addition to the exonucleases described above, recBCD nuclease is responsible for degradation of linear dsDNA in E. coli. Efforts to knock out recBCD in DH5alpha ΔrecJ ΔxonA background to investigate the effect of this nuclease on SCRIBE efficiency were unsuccessful, suggesting that the combination of these mutations are likely to be unviable in this background.

FIGS. 11A-11C: Mapping the connectome for conjugative mating pairs in a bacterial population. (FIG. 11A) Recording pairwise interactions (conjugation events) between conjugative pairs of bacteria using SCRIBE-based DNA memory. Interactions between a recipient cell and donor cell are recorded into neighboring DNA memory registers in the recipient cell genome (FIG. 11B) Number of unique variants (interactions) per million reads obtained from sequencing DNA registers in genomes of recipient cells after conjugation with donor cells. Unique variants in the SCRIBE-targeted registers (both Register 1 and Register 2) were three orders of magnitude higher than in randomly chosen non-targeted registers, indicating successful recording of conjugation events. (FIG. 11C) The connectivity matrix as well as the corresponding interaction subnetwork for the first 20 (alphabetically sorted) barcodes of donors and recipients in one of the samples are shown. The y- and x-axis show recipient genomic barcodes (recorded in Register 1) and donor barcodes (recorded in Register 2), respectively. Boxes depict connected barcodes, indicating that a conjugation event from the corresponding donor resulted in SCRIBE transfer and subsequent recording of the donor barcode into the specific recipient genome. In the interaction network shown, donor and recipient barcodes are indicated by dark gray (“d-barcode”) and light gray (“r-barcode”) rectangles, respectively. (FIG. 11D) Schematic representation of the barcode joining strategy used to record pairwise interactions (conjugation events) between conjugative pairs of bacteria using HiSCRIBE-based DNA writing. Upon successful conjugation, the interactions between a recipient cell and donor cell are recorded into neighboring DNA memory registers in the recipient cell genome. The edited registers are then amplified using allele-specific PCR (to deplete non-edited registers) and the identity of the interacting partners are retrieved by sequencing. A single nucleotide that was included in each barcode to distinguish between unedited and edited registers. These “writing control” nucleotides were then used to selectively amplify edited registers by allele-specific PCR using primers that match to these nucleotides but not to unedited registers. (FIG. 11E) Detecting the spatial organization of bacterial populations. Donor and recipient bacterial populations harboring δHiSCRIBE-encoded “d-barcode” (dark grey circles) and “r-barcode” (light grey circles), respectively, were spotted on nitrocellulose filters that were then placed on agar surface in the patterns shown in the left panel. Conjugation mixtures were harvested and the memory registers were amplified by allele-specific PCR and sequenced by Illumina sequencing (see Methods). Recorded barcodes in the two consecutive memory registers were parsed and the donor-recipient population connectivity matrix was calculated based on the percentage of reads corresponding to each possible pair-wise interaction of donors and recipient barcodes. The heatmap representation of the retrieved connectivity matrix (middle panel) as well as the corresponding interaction network (right panel) are shown. Light grey boxes in the heatmap depict connected barcodes, indicating that a conjugation event from the corresponding donor resulted in δHiSCRIBE transfer and subsequent recording of the donor barcode into the specific recipient genome. In the interaction network, donor and recipient barcodes are indicated by dark gray (“d-barcode”) and light gray (“r-barcode”) rectangles, respectively. (FIG. 11F) Conjugation donor and recipient cells harboring δHiSCRIBE-encoded “d-barcode” and “r-barcodes” were spotted on nitrocellulose filters placed on agar surface as indicated by circles, respectively. These plasmids were designed to introduce unique 6 bp barcodes, as well as additional mismatches (which serve as “writing control nucleotides” to discriminate between edited and unedited memory registers when selectively PCR amplifying the edited registers) into two adjacent memory register on the galK locus, once inside the recipient cells. Samples taken from the intersection of the donor and recipient populations were lysed and used as templates in allele-specific PCR. Allele-specific PCR using primers that bind to the “writing control nucleotides” (but not to the non-edited registers) was used to selectively amplify the edited registers and deplete non-edited registers. The identities of the two barcodes corresponding to the interacting donor and recipient populations were then retrieved by Sanger sequencing. The sequences from top to bottom, correspond to SEQ ID NOs: 46 and 47. (FIG. 11G) Additional examples of cellular patterns that were recorded by the barcode joining approach described in FIG. 11D. Their corresponding weighted connectivity matrices and interaction networks that were faithfully retrieved using high-throughput sequencing.

FIG. 12: Mapping cellular connectomes by DNA sequencing.

FIG. 13: Mapping transient interactions by dynamic genome engineering followed by DNA sequencing.

FIGS. 14A and 14B: A model for HiSCRIBE-mediated recombineering. (FIG. 14A) Genome editing efficiencies of SCRIBE harboring a catalytically inactive reverse transcriptase (dRT, in which the conserved YADD motif in the active site of the RT is replaced with YAAA) was determined by the kanR reversion assay in different knockout backgrounds. Error bars indicate standard error of the mean for three biological replicates. (FIG. 14B) Proposed model for retron-mediated recombineering. Intracellular recombinogenic oligonucleotides are likely generated due to degradation of template plasmid as well as msDNA (retron product). ssDNA-specific cellular exonucleases (XonA and RecJ) can process these oligonucleotides into smaller, non-recombinogenic (oligo)nucleotides. Alternatively, Beta can bind to, protect, and recombine these oligonucleotides into their genomic target loci. (FIG. 14C) Effect of ssDNA homology length on HiSCRIBE DNA writing efficiency. Different δHiSCRIBE(kanR)_{ON} plasmids expressing ssDNAs with different lengths of homology to the kanR_{OFF} target were tested by the kanR reversion assay in DH5αPRO ΔrecJ ΔxonA kanR_{OFF} reporter strain. Maximal editing efficiency was observed with ssDNAs encoding 35 bp homology arms. Error bars indicate standard errors for three biological replicates.

### DETAILED DESCRIPTION

Provided herein, in some embodiments, are genetically-encoded genomic editing systems (including, e.g., nucleic acid constructs, methods, cells, and kits) that enable efficient, autonomous and dynamic editing (writing) of bacterial genomes within bacterial communities, which may be expanded to genetically intractable organisms. These systems permit a selective increase in the rate of incorporation of (pre-defined) mutations to specific regions of a bacterial genome, for example, more than eight orders of magnitude over the background mutation rate. These systems can be delivered to subpopulations of host cells within a larger resident community via various delivery mechanisms. Following delivery, the systems can be coupled to host (natural or synthetic) cell regulatory circuits, for example, for single-cell computation and memory applications.

The high-efficiency genome editing systems, as provided herein, may be coupled to continuous delivery systems, thus enabling autonomous and continuous diversification of desired genomic loci. Such coupled system can then be combined with continuous selection/screening system, permitting continuously modification and selection of a trait of interest. Thus, the genome editing systems of the present disclosure may be used to selectively increase de novo mutation rate of desired genomic loci while minimizing background mutation rate, thereby evolving specific segments of a genome in a controlled, tunable manner.

While recent advances in genomic engineering technologies have enabled, to some extent, targeted modifications of bacterial genomes, the existing platforms are limited to a few laboratory model strains and specific conditions and often suffer from suboptimal editing efficiencies. As such, they can only be used under laboratory conditions and are not suitable to be applied in situ (in the context of natural bacterial communities). The genomic editing systems of the present disclosure, by contrast, are scalable system that enable continuous and dynamic manipulation of genomic DNA at nucleotide precision and with high efficiency. The systems, as provided herein, can be integrated with cellular regulatory networks and can autonomously respond to cellular cues, thus enabling the production of evolvable and self-sustainable cells and communities that can autonomously rewrite and tune their genomic make up over time in response to environmental cues (evolve). The systems also enable the production of cells that, under a suitable selective pressure, may undergo accelerated evolution toward desired evolutionary paths. The ability to selectively increase mutation rates of specific segments of a genome connected to a phenotype of interest (while preserving the background (global) mutation rate at the minimal level) may provide selective advantages to an organism for adaptation.

### Genomic Editing Constructs

SCRIBE (Synthetic Cellular Recorders Integrating Biological Events) is a platform for recording analog information into genomic DNA based on conditional and targeted genome editing of bacterial genome by in vivo expression of single-stranded DNA followed by recombineering (Farzadfard, T. K. Lu, Science 346, 1256272 (2014), incorporated herein by reference). The genomic editing constructs described herein enable high efficiency genome editing in any genetic background, including wild type genetic background with a fully active mismatch repair system (MMR). This is significant because it enables editing of a bacterial genome that cannot be otherwise manipulated, e.g., a bacterial genome within a bacterial community. In some embodiments, the high efficiency SCRIBE platform is also referred to herein as “HiSCRIBE.”

The high recombination efficiency of the genomic editing constructs of the present disclosure rely on the removal from the bacterial cell factors that limit their efficiency. Factors that limit the efficiency of current genome editing systems have been identified, e.g., the MMR and cellular exonucleases such as RecJ, XonA, and ExoX. Thus, the genomic editing constructs of the present disclosure, in some embodiments, contain genetic elements that downregulate these factors. In some embodiments, the exonuclease (e.g., RecJ, XonA, or ExoX) are knocked out from the genome of the bacterial cell harboring the SCRIBE platform. High efficiency SCRIBE (HiSCRIBE) in a nuclease knockout background is also herein referred to as the “δHiSCRIBE system.” In some embodiments, conditional knockout of the nucleases (e.g., RecJ, XonA, or ExoX) is achieved using the CRISPRi technology (e.g., as described in Qi et al., Repurposing CRISPR as an RNA-Guided Platform for Sequence-Specific Control of Gene Expression, Cell. 2013 Feb. 28; 152(5): 1173-1183, incorporated herein by reference). High efficiency SCRIBE (HiSCRIBE) in a conditional nuclease knockout background using CRISPRi is also herein referred to as the “χHiSCRIBE” system.

The genomic editing constructs described herein is an engineered nucleic acid construct. An “engineered nucleic acid construct” refers to an engineered nucleic acid having multiple genetic elements. Engineered nucleic acid constructs of the present disclosure, in some embodiments, include a promoter operably linked to a nucleic acid that comprises: (a) a nucleotide sequence encoding a guide RNA targeting an exonuclease; (b) a nucleotide sequence encoding a single-stranded msrRNA and a single-stranded msdDNA modified to contain a targeting sequence; and (c) a nucleotide sequence encoding a reverse transcriptase protein, wherein (b) is flanked by a pair of inverted repeat sequences. In some embodiments, the constructs also include a nucleotide sequence that encodes a Cas9 protein (e.g., a Streptococcus pyogenes Cas9). In some embodiments, the Cas9 protein may be an activate Cas9 nuclease. In some embodiments, the Cas9 protein may be a catalytically-inactive Cas9 (dCas9). In some embodiments, the constructs also include a nucleotide sequence that encodes a single-stranded DNA (ssDNA)-annealing recombinase protein (e.g., a Beta recombinase protein or a Beta recombinase protein homolog). The engineered nucleic acid construct may also comprise one or more additional elements, e.g., promoters, stop codons, and/or nucleotide sequences encoding one or more ribozymes.

The genomic editing constructs of the present disclosure, in some embodiments, include nucleotide sequences encoding a guide RNA, a msdDNA, a msrRNA and a reverse transcriptase, which enables dual-function genomic editing: oligonucleotide recombineering and CRISPR/Cas9-mediated targeted genetic manipulation. Thus, some aspects of the present disclosure are directed to engineered nucleic acid constructs that comprise nucleotide sequences encoding the CRISPR/Cas9 elements, e.g., guide RNAs, and/or Cas9 protein. The S. pyogenes Clustered Regularly-Interspaced Short Palindromic Repeats and CRISPR associated 9 (CRISPR/Cas9) system is an effective genome engineering system. The Cas9 protein is a nuclease that catalyzes double-stranded breaks and generates mutations at DNA loci targeted by a small guide RNA (sgRNA or simply gRNA). A “guide RNA,” as used herein, refers to a nucleotide sequence that can target (i.e., guide) a programmable nuclease (e.g., Cas9 or dCas9) to its target sequence. The native gRNA is comprised of a 20 nucleotide (nt) Specificity Determining Sequence (SDS), which specifies the DNA sequence to be targeted, and is immediately followed by a 80 nt scaffold sequence, which associates the gRNA with Cas9. In some embodiments, the SDS is about 20 nucleotides long. For example, the SDS may be 15, 16, 17, 18, 19, 20, 21, 22, 23, 24, or 25 nucleotides long. At least a portion of the target DNA sequence needs to be complementary to the SDS of the gRNA. For Cas9 to successfully bind to the target DNA sequence, a region of the target DNA sequence must be complementary to the SDS of the gRNA sequence and must be immediately followed by the correct protospacer adjacent motif (PAM) sequence (e.g., “NGG”). In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence.

When a gRNA “targets” a target sequence, e.g., a sequence in a genome, the SDS in the gRNA binds to the target sequence via sequence complementarity, and the Cas9 associated with the gRNA in the scaffold sequence also binds to the target sequence. Upon binding to the target DNA sequence, a wild type Cas9 introduces a double-stranded break in the target DNA locus. When the double-strand break is introduced in a eukaryotic genome, the break is repaired by either homologous recombination (when a repair template is provided) or error-prone non-homologous end joining (NHEJ) DNA repair mechanisms, resulting in mutagenesis (e.g., nucleotide deletions or insertions) of the targeted locus. In contrast, a double-stranded break introduced by Cas9-gRNA complex in a bacterial genome may not be repaired, leading to bacterial cell death.

In some embodiments, the Cas9 protein that may be used in accordance with the present disclosure is a catalytically-inactive Cas9 (dCas9). Unlike wild type Cas9 nuclease, upon binding to the target DNA sequence, the dCas9 does not introduce a double-stranded DNA break. However, in some embodiments, the binding of dCas9 to the target DNA sequence may exclude the binding of other proteins to the target DNA sequence via steric hindrance. Thus, for example, if the target DNA sequence is located in a regulatory region of a gene, binding of the dCas9-gRNA complex to the target DNA sequence prevents the binding of transcriptional regulators, e.g., a transcription activator or a transcription suppressor, thus modulating gene expression (also referred to as “CRISPRi,” Qi et al., Repurposing CRISPR as an RNA-Guided Platform for Sequence-Specific Control of Gene Expression, Cell. 2013 Feb. 28; 152(5): 1173-1183, incorporated herein by reference).

In some embodiments, the gRNA encoded by the genomic editing constructs of the present disclosure targets bacterial cellular genes that reduce genomic editing efficiency, e.g., mismatch repair system (MMR) factors (e.g., mutS) and exonucleases (e.g., recJ, xonA, exoX, etc.). In some embodiments, the gRNA targets the mutS gene. In some embodiments, the gRNA targets a bacterial cellular exonuclease. In some embodiments, the gRNA targets the recJ gene. In some embodiments, the gRNA targets the xonA gene. In some embodiments, the gRNA targets the exoX gene. In some embodiments, the genomic editing constructs described herein comprises nucleotide sequences encoding more than one gRNAs. For example, the genome-editing construct may comprise nucleotide sequences encoding 2, 3, 4, 5, or more gRNAs. In some embodiments, the genome-editing construct comprises a nucleotide sequence encoding a gRNA targeting the recJ gene and a nucleotide sequence encoding a gRNA targeting the xonA gene. In some embodiments, the genome-editing construct comprises a nucleotide sequence encoding a gRNA targeting the recJ gene, a nucleotide sequence encoding a gRNA targeting the xonA gene, and a nucleotide sequence encoding a gRNA targeting the exoX gene.

In some embodiments, the genome-editing construct described herein further comprises a nucleotide sequence encoding a Cas9 protein. In some embodiments, the CRISPR/Cas9 elements are used herein to disrupt (e.g., reduce or knockdown) the expression of bacterial cellular exonucleases. As such, in some embodiments, the genome-editing construct comprises a nucleotide sequence encoding a catalytically inactive Cas9 (dCas9) protein. In some embodiments, the nucleotide sequence encoding a dCas9 may encode the S. pyogenes dCas9 protein comprising the amino acid sequence of SEQ ID NO: 1. Compare to the wild-type S. pyogenes Cas9 protein, the S. pyogenes dCas9 protein comprises a D10A and a H840A mutation. In some embodiments, the nucleotide sequence encoding a dCas9 may encode a homolog of the S. pyogenes dCas9 comprising an amino acid sequence that is at least 70%, at least 80%, at least 85%, at least 90%, at least 95%, or at least 99% identical to SEQ ID NO: 1, and comprising mutations corresponding to the D10A and H840A mutations in SEQ ID NO: 1.

S. pyogenes dCas9 sequence
(SEQ ID NO: 1)
MDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD
(single underline: D10A and H840A mutation)

To target the binding of the dCas9 to the exonuclease genes and disrupt their expression (e.g., using CRISPRi), in some embodiments, the gRNA may target a regulatory region upstream of the said genes.

When the target genes, e.g., the bacterial cellular exonucleases, are targeted by the gRNA-dCas9 complexes, the expression level of the proteins encoded by these genes reduces. In some embodiments, the expression level or activity (i.e., exonuclease activity) level may be reduce by at least 30%. For example, the expression level may be reduced by at least 30%, at least 40%, at least 50%, at least 60%, at least 70%, at least 80%, at least 90%, at least 95%, at least 99%, or more. In some embodiments, the expression level or activity (i.e., exonuclease activity) level may be reduced by 100%. As such, the remaining protein level or activity (i.e., exonuclease activity) level in the bacterial cell may be no more than 70%, no more than 60%, no more than 50%, no more than 40%, no more than 30%, no more than 20%, no more than 10%, no more than 5%, no more than 1%, or less as compared to that of cells without the gRNA-Cas9 complexes. In some embodiments, the remaining protein level or activity (e.g., exonuclease activity) level in the bacterial cell may be 0% as compared to that of cells without the gRNA-dCas9 complexes.

In some embodiments, the CRISPR/Cas9 elements in the engineered nucleic acid construct of the present disclosure (e.g., see FIG. 1E) may be used to target an unmodified version of a target sequence, e.g., an undesired allele of a gene, to counter select against the unmodified target sequence and enhance the genomic editing efficiency. In these instances, the gRNA may be designed to target the unmodified target sequence and a wild type Cas9 nuclease may be used such that when the Cas9 nuclease is targeted to the unmodified target sequence, it introduces a double-strand DNA break, leading to bacterial cell death. In contrast, cells that contain a target sequence that is modified via recombineering will not be targeted.

In some embodiments, the nucleotide sequence encoding a wild type Cas9 may encode the wild-type S. pyogenes Cas9 comprising the amino acid sequence of SEQ ID NO: 2. In some embodiments, the nucleotide sequence encoding a wild type Cas9 may encode a homolog of the S. pyogenes Cas9 comprising an amino acid sequence that is at least 70%, at least 80%, at least 85%, at least 90%, at least 95%, or at least 99% identical to SEQ ID NO: 2.

Wild Type Cas9 nuclease sequence
(SEQ ID NO: 2)
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

In some embodiments, the genomic editing construct described herein may be transcribed into a polycistronic mRNA, e.g., when all genetic elements in the construct are placed downstream of one promoter. A “polycistronic mRNA” refers to a messenger RNA which encodes two or more end products, e.g., gRNAs and proteins. For gRNAs to guide the Cas9 protein (e.g., dCas9) to its target sequence, it needs to be released from the polycistronic mRNA. Thus, some aspects of the present disclosure provide genetic elements that allow the release of the gRNAs from the polycistronic mRNA upon its transcription. In some embodiments, the said genetic element is a ribozyme. A “ribozyme” refers to a ribonucleic acid (RNA) enzyme that catalyzes a chemical reaction. The ribozyme catalyzes specific reactions in a similar way to that of protein enzymes. Some ribozymes have been found to be able to cleave itself from the rest of the mRNA it is transcribed in, e.g., the hammerhead ribozyme (HHR) or the hepatitis delta virus ribozyme (HDVR). In some embodiments, a nucleotide sequence encoding the ribozyme is inserted between each nucleotide sequence encoding a gRNA and the next genetic element in the construct, i.e., downstream (e.g., toward the 3′ end) of the nucleotide sequence encoding the gRNA but upstream (e.g., toward the 5′ end) of the nucleotide sequence encoding the next genetic element. In some embodiments, the ribozyme is a hammerhead ribozyme. In some embodiments, the ribozyme is a hepatitis delta virus ribozyme. In some embodiments, more than one ribozymes may be used. For example, a nucleotide sequence encoding both hammerhead ribozyme (HHR) and the hepatitis delta virus ribozyme (HDVR) may be inserted between each nucleotide sequence encoding the gRNA and the next genetic element in the construct. In some embodiment, the HDVR is upstream of the HHR, while in other embodiments, the HHR is upstream of the HDVR.

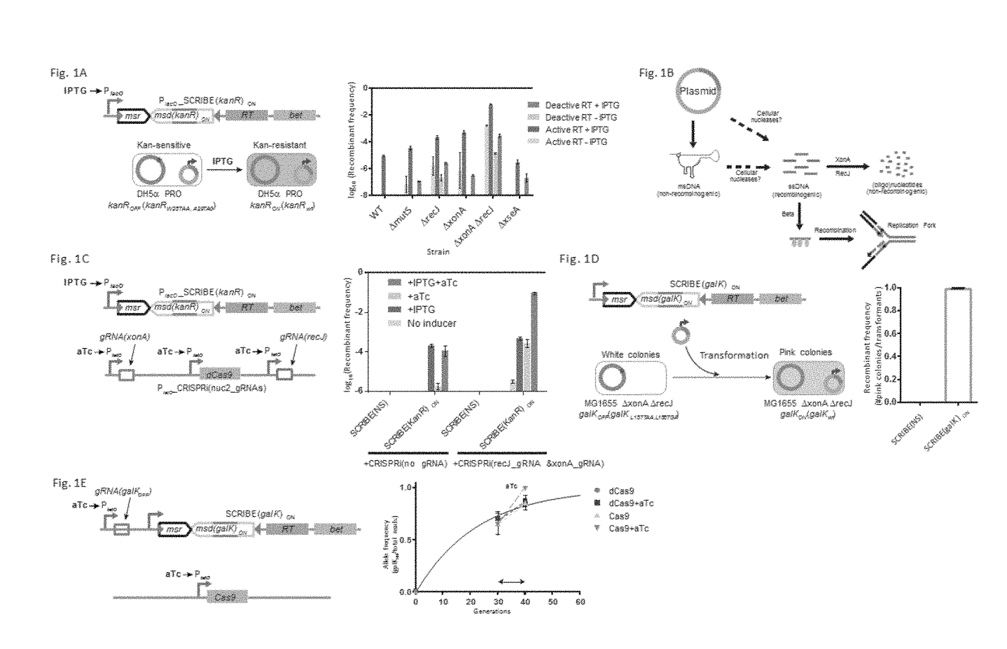

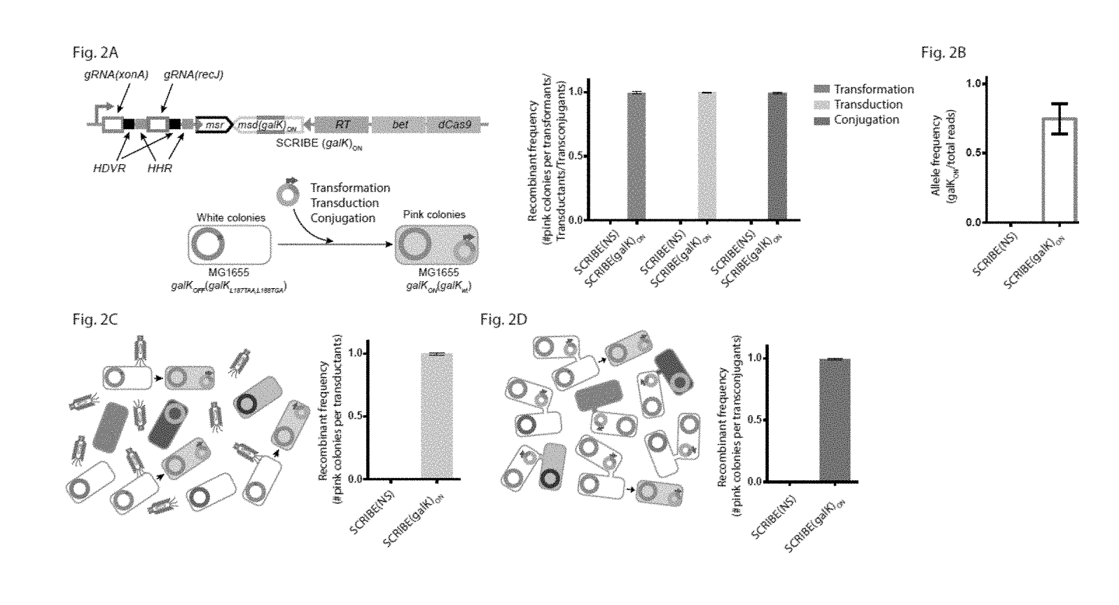

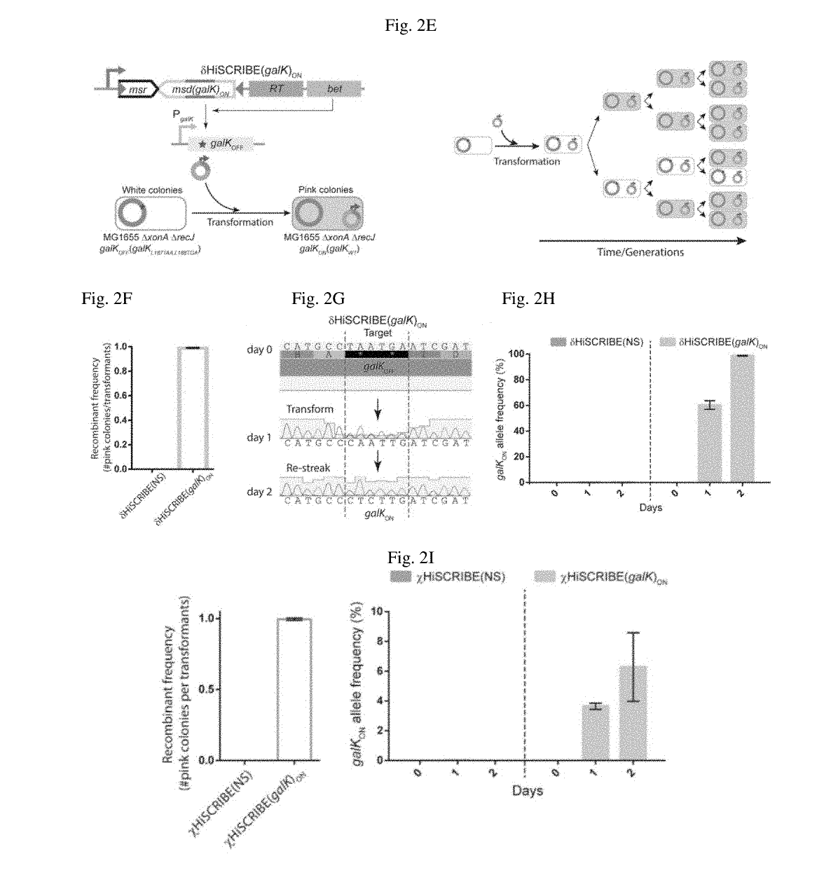

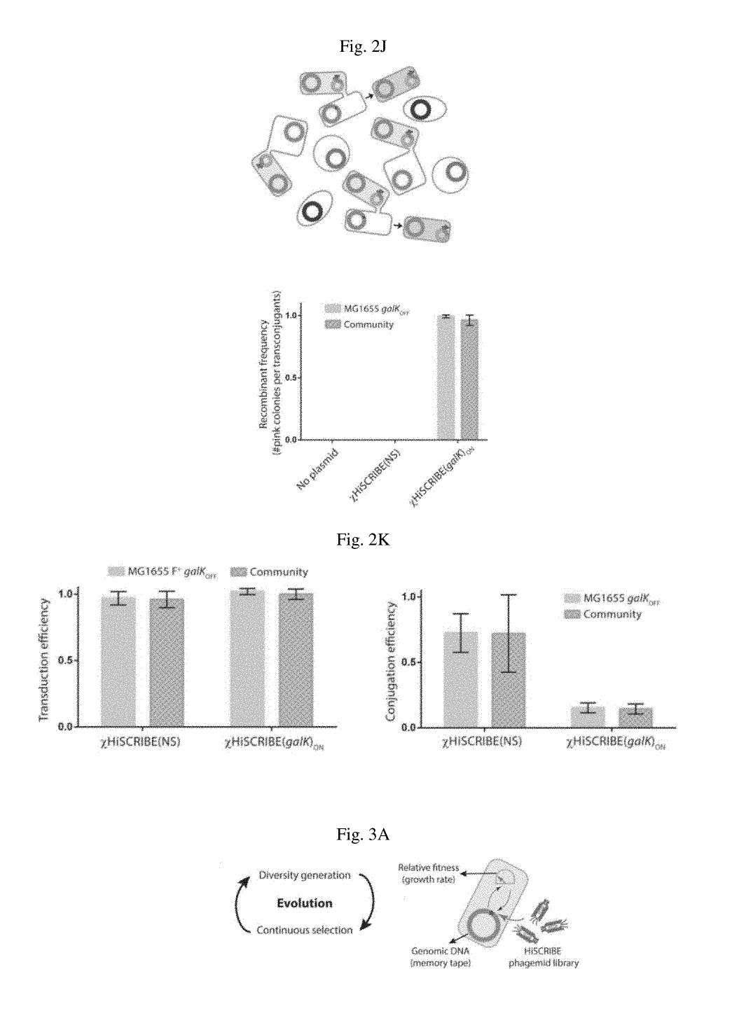

In addition to the CRISPR/Cas9 elements, the genomic editing construct of the present disclosure further comprises elements for ssDNA-mediated recombineering, which are adapted from the bacterial retron elements including an msdDNA, an msrRNA, and a reverse transcriptase. A wild-type (e.g., unmodified) retron is a type of prokaryotic retroelement responsible for the synthesis of small extra-chromosomal satellite DNA referred to as multicopy single-stranded (ms) DNA. A wild-type msdDNA is composed of a small, single-stranded DNA, bound to a small, single-stranded RNA. Internal base pairing creates various stem-loop/hairpin secondary structures in the msdDNA. The msr-msd sequence in the retron is flanked by two inverted repeats (FIG. 2A, gray triangles). Once transcribed, the msr-msd RNA folds into a secondary structure guided by the base-pairing of the inverted repeats and the msr-msd sequence. The RT recognizes this secondary structure and uses a conserved guanosine residue in the msr as a priming site to reverse transcribe the msd sequence and produce a hybrid ssRNA-ssDNA molecule referred to as msdDNA. It is known that the middle part of the msd sequence is dispensable and can be replaced with a template to produce ssDNAs of interest (e.g., see FIG. 2A, (galK)_{ON}) in vivo.

Thus, in some embodiments, the genomic editing construct of the present disclosure comprises a nucleotide acid sequence encoding a single-stranded msrRNA and a single-stranded msdDNA modified to contain a targeting sequence, and a nucleotide sequence encoding a reverse transcriptase. A “targeting sequence” refers to a nucleotide sequence (e.g., DNA) within a single-stranded msd DNA that is complementary or partially complementary to a target sequence (e.g., genomic sequence). A targeting sequence, when bound by a ssDNA-annealing recombinase, anneals to and recombines with its target sequence. A “target sequence” may be, for example, located genomically in a cell or otherwise present in a cell (e.g., located on an episomal vector).

In some embodiments, a targeting sequence has a length of at least 15 nucleotides. For example, a targeting sequence may have a length of 15 to 100 nucleotides, or 15 to 200 nucleotides, or more. In some embodiments, a targeting sequence has a length of 15 to 50, 15 to 60, 15 to 70, 15 to 80, or 15 to 90 nucleotides. In some embodiments, a targeting sequence has a length of 20 to 50, 20 to 60, 20 to 70, 20 to 80, 20 to 90, or 20 to 100 nucleotides.

In some embodiments, a targeting sequence comprises at least 15 nucleotides (e.g., contiguous nucleotides) that are complementary to a target genomic sequence of a cell into which an engineered nucleic acid construct containing the targeting sequence has been delivered. In some embodiments, a targeting sequence comprises at least 20, at least 30, at least 40, at least 50, at least 60, at least 70, at least 80, at least 90, or at least 100 nucleotides (e.g., contiguous nucleotides) that are complementary a target genomic sequence of a cell into which an engineered nucleic acid construct containing the targeting sequence has been delivered. In some embodiments, a targeting sequence comprises 15 to 100, 15 to 90, 15 to 80, 15 to 70, 15 to 60, 15 to 50, 15 to 40, or 15 to 30 nucleotides (e.g., contiguous nucleotides) that are complementary to a target genomic sequence of a cell into which an engineered nucleic acid construct containing the targeting sequence has been delivered.

In some embodiments, a targeting sequence is 100% complementary to its target sequence. In some embodiments a targeting sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence. Such a targeting sequence with partially complementarity to its target sequence may be used, for example, to introduce mutations or other genetic changes (e.g., genetic elements such as stop codons) into its target sequence.

The nucleotide sequence encoding the msrRNA and the msdDNA is flanked by a pair of inverted repeat sequences. An “inverted repeat sequence” is a sequence of nucleotides followed upstream (e.g., toward the 5′ end) or downstream (e.g., toward the 3′ end) by its reverse complement. Inverted repeat sequences of the present disclosure typically flank an msr-msd sequence in a retron and, once transcribed, binding of the two sequences guides folding of the transcribed molecule into a secondary structure. Inverted repeat sequences are typically specific for each retron. For example, an inverted repeat sequence for the wild-type retron Ec86 (or for genetic elements obtained from the type retron Ec86) is TGCGCACCCTTA (SEQ ID NO: 3). In some embodiments, the length of an inverted repeat sequence is 5 to 15, or 5 to 20 nucleotides. For example, the length of an inverted repeat sequence may be 5, 6, 7, 8, 9, 10, 11, 12, 13, 14, 15, 16, 17, 18, 19 or 20 nucleotides. In some embodiments, the length of an inverted repeat sequence is longer than 20 nucleotides.

A “reverse transcriptase (RT)” is an enzyme used to generate complementary DNA from an RNA template. Reverse transcriptases may be obtained from prokaryotic cells or eukaryotic cells. Reverse transcriptases of the present disclosure are used to reverse transcribe template msd RNA into single-stranded msdDNA. In some embodiments, a reverse transcriptase is encoded by a retron ret gene. Other examples of reverse transcriptases (RTs) that may be used in accordance with the present disclosure include, without limitation, retroviral RTs (e.g., eukaryotic cell viruses such as HIV RT and MuLV RT), group II intron RTs and diversity generating retroelements (DGRs).

Recombination of ssDNA produced in vivo may be mediated by a ssDNA-annealing recombinase protein. Thus, the genome-editing construct of the present disclosure may further comprise nucleotide acid sequences encoding a single-stranded DNA (ssDNA)-annealing recombinases such as, for example, Beta recombinase protein (e.g., encoded by the bacteriophage lambda bet gene) or a homolog thereof. When expressed in cells (e.g., bacterial cells such as Escherichia coli cells) ssDNA-annealing recombinases mediate ssDNA recombination. The term “recombination” refers to the process by which two nucleic acids exchange genetic information (e.g., nucleotides). Non-limiting examples of ssDNA-annealing recombinases for use in accordance with the present disclosure include recombinases obtained from bacteriophages or prophages of Gram-positive bacteria Bacillus subtilis, Mycobacterium smegmatis, Listeria monocytogenes, Lactococcus lactis, Staphylococcus aureus, and Enterococcus faecalis as well as from the Gram-negative bacteria Vibrio cholerae, Legionella pneumophila, and Photorhabdus luminescens (S. Datta, et al. PNAS 105, 1616-1631 (2008)). Specific examples of recombinases for use as provided herein include, without limitation, those listed in Table 1.

TABLE 1
ssDNA- Annealing Recombinase Proteins (table 5 of earlier application)

| Original | | Accession | | Recombinase (R) Exonuclease |
|---|---|---|---|---|
| Host | Source | Number | Nucleotide | (E) genes and promoter (P) |
| bet/exo | Phage lambda; E. coli | NIH collection | NC_001416 | 32025-32810/31348-32028 |
| s065/s066 | SXT element; Vibrio | D. I. Friedman | AY055428 | 72817-73635/73921-74937 |
| | cholerae | | | |
| plu2935/ | Photorhabdus | A. Danchin | BX571868 | 324693-325613/325614-326297 |
| plu2936 | luminescens | | | |
| EF2132/ | Enterococcus faecalis | S. L. Adhya | AE016830 | 2041370-2042293/2040592-2041404 |
| EF2131 | | | | |
| recT/recE | Rac prophage; E. coli | NIH collection | NC_000913 | 1412008-1412817/1412810-1415410 |
| orfC/orfB | Legionella pneumophila | E. Lüneberg | AJ277755 | 1415-2299/560-1402 |
| gp35/gp34.1 | Phage SPP1; Bacillus | S. Moineau | X97918 | 32175-33038/30532-31467 |
| | subtilis | | | |
| gp61/gp60 | Phage Che9c; | G. Hatfull | AY129333 | 43643-44704/42706-43650 |
| | Mycobacterium | | | |
| | smegmatis | | | |
| orf48/orf47 | Phage A118; Listeria | R. Calender | AJ242593 | 32773-33588/31811-32770 |
| | monocytogenes | | | |
| orf245/- | Phage ul36.2; | S. Moineau | AF212847 | 1678-2415 |
| | Lactococcus lactis | | | |
| gp20/- | Phage phiNM3; | T. Bae | NC_008617 | 10317-11237 |
| | Staphylococcus aureus | | | |

Bacteriophage lambda Red Beta recombinase protein (referred to herein as “Beta recombinase”) mediates recombination-mediated genetic engineering, or “recombineering,” using ssDNA. Unlike recombineering with double-stranded DNA, recombineering with ssDNA does not require other bacteriophage lambda red recombination proteins, such as Exo and Gamma. Beta recombinase binds to ssDNA and anneals to the ssDNA to complementary ssDNA such as, for example, complementary genomic DNA. It can efficiently recombine linear DNA with homologs as short, for example, 20-70 bases (N. Constantino et al., Proc Natl Acad Sci USA 100(26): 15748-53 (2003)). Thus, in some embodiments, as discussed above, a targeting sequence has a length of 20 to 70 nucleotides. As used herein, the term “Beta recombinase,” in some embodiments, may include Beta recombinase homologs (S. Datta, et al. Proc Natl Acad Sci USA 105: 1626-1631 (2008)), in addition to the recombinases listed in Table 1.

In some embodiments, the CRISPR elements and the recombineering elements of a genomic editing construct described herein are arranged such that a promoter is located upstream of a nucleotide sequence encoding an gRNA, which is upstream of the nucleotide sequence encoding the msrRNA and the modified msdDNA, which is upstream of the nucleotide sequence encoding the reverse transcriptase, which is upstream of a nucleotide sequence encoding an ssDNA recombinase, which is upstream of a nucleotide sequence encoding the Cas9 protein (e.g., an active Cas9 nuclease or a dCas9), wherein the nucleotide sequence encoding the msrRNA and the modified msdDNA is flanked by a pair of inverted repeat sequences (FIG. 2A). That is, in some embodiments, the genetic elements of an engineered nucleic acid construct are arranged in the following 5′ to 3′ orientation: promoter, gRNA sequence and ribozyme sequences, optionally a second gRNA sequence and ribozyme sequences, optionally third gRNA sequence and ribozyme sequences, inverted repeat sequence, nucleotide sequence encoding a single-stranded msr RNA, nucleotide sequence encoding a single-stranded msdDNA, inverted repeat sequence, nucleotide sequence encoding a reverse transcriptase protein, nucleotide sequence encoding an ssDNA recombinase, and nucleotide sequence encoding a Cas9 protein. It should be understood that each “inverted repeat sequence” is one of a pair of inverted repeat sequences that are complementary to each other and bind to each once transcribed so as to assist in folding of the transcribed RNA into a secondary structure.

In some embodiments, the gRNA encoding sequences, the recombineering elements, or the Cas9 protein are operably linked to different promoters. For example, in some embodiments, the nucleotide sequence encoding one or more gRNAs may be operably linked to a first promoter, the nucleotide sequence encoding the recombineering elements (e.g., the msrRNA, the msdDNA, and the RT) is operably linked to a second promoter, and the nucleotide sequence encoding the Cas9 protein is operably linked to a third promoter, wherein the first promoter, the second promoter, and the third promoter are different from one another.

In some embodiments, the genetic elements of a genome-editing construct are arranged on separate nucleic acids. For example, the gRNAs and the recombineering elements may be encoded on separate nucleic acids. Similarly, the msrRNA and msdDNA may be encoded on separate nucleic acids as the reverse transcriptase. Or, the gRNAs and the recombineering elements may be on one nucleic acid construct, while the Cas9 protein is encoded on a different nucleic acid construct, and the ssDNA recombinase is encoded one yet another nucleic acid construct. It is to be understood that when different genetic elements are encoded on separate nucleic acid constructs, each genetic element on its own construct is operably linked to a promoter.

A “nucleic acid” refers to at least two nucleotides covalently linked together, and in some instances, may contain phosphodiester bonds (e.g., a phosphodiester “backbone”). In some embodiments, a nucleic acid (e.g., an engineered nucleic acid) of the present disclosure may be considered a nucleic acid analog, which may contain other backbones comprising, for example, phosphoramide, phosphorothioate, phosphorodithioate, O-methylphophoroamidite linkages, and/or peptide nucleic acids. Nucleic acids (e.g., components, or portions, of the nucleic acids) of the present disclosure may be naturally occurring or engineered. Nucleic acids of the present disclosure may be single-stranded (ss) or double-stranded (ds), as specified, or may contain portions of both single-stranded and double-stranded sequence (e.g., a single-stranded nucleic acid with stem-loop structures may be considered to contain both single-stranded and double-stranded sequence). It should be understood that a double-stranded nucleic acid is formed by hybridization of two single-stranded nucleic acids to each other. Nucleic acids may be DNA, including genomic DNA and cDNA, RNA or a hybrid/chimeric of any two or more of the foregoing, where the nucleic acid contains any combination of deoxyribonucleotides and ribonucleotides, and any combination of bases, including uracil, adenine, thymine, cytosine, guanine, inosine, xanthine, hypoxanthine, isocytosine, and isoguanine.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Recombinant nucleic acids and synthetic nucleic acids also include those molecules that result from the replication of either of the foregoing. Engineered nucleic acid constructs of the present disclosure may be encoded by a single molecule (e.g., included in the same plasmid or other vector) or by multiple different molecules (e.g., multiple different independently-replicating molecules).

Engineered nucleic acid constructs of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ′Y extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

Engineered nucleic acid constructs of the present disclosure may be included within a vector, for example, for delivery to a cell. A “vector” refers to a nucleic acid (e.g., DNA) used as a vehicle to artificially carry genetic material (e.g., an engineered nucleic acid construct) into a cell where, for example, it can be replicated and/or expressed. In some embodiments, a vector is an episomal vector (see, e.g., Van Craenenbroeck K. et al. Eur. J. Biochem. 261, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector.

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof.

A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. No. 4,683,202 and U.S. Pat. No. 5,928,906). Examples of promoters for use in accordance with the present disclosure include, without limitation, Piac0, Pteto, PiuxR, PλM and PfixK2. Other promoters are described below.

Promoters of an engineered nucleic acid construct may be “inducible promoters,” which refer to promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

In some embodiments, inducible promoters of the present disclosure function in prokaryotic cells (e.g., bacterial cells). Examples of inducible promoters for use prokaryotic cells include, without limitation, bacteriophage promoters (e.g. Pis Icon, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), aS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLac01, dapAp, FecA, Pspac-hy, pel, plux-cl, plux-lac, CinR, CinL, glucose controlled, modified Pr, modified Prm+, FecA, Pcya, rec A (SOS), Rec A (SOS), EmrR_regulated, Bet1_regulated, pLac_lux, pTet_Lac, pLac/Mnt, pTet/Mnt, LsrA/cI, pLux/cI, Lac1, LacIQ, pLacIQ1, pLas/cI, pLas/Lux, pLux/Las, pRecA with LexA binding site, reverse BBa_R0011, pLacI/ara-1, pLacIq, rrnB PI, cadC, hns, PfhuA, pBad/araC, nhaA, OmpF, RcnR), aS promoters (e.g., Lutz-Bujard LacO with alternative sigma factor σ38), σ32 promoters (e.g., Lutz-Buj ard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and σB promoters. Other inducible microbial promoters may be used in accordance with the present disclosure.

In some embodiments, inducible promoters of the present disclosure function in eukaryotic cells (e.g., mammalian cells). Examples of inducible promoters for use eukaryotic cells include, without limitation, chemically-regulated promoters (e.g., alcohol-regulated promoters, tetracycline-regulated promoters, steroid-regulated promoters, metal-regulated promoters, and pathogenesis-related (PR) promoters) and physically-regulated promoters (e.g., temperature-regulated promoters and light-regulated promoters).

### Cells

Other aspects of the present disclosure provide cells that comprise any of the engineered nucleic acid constructs described herein, e.g., the genomic editing construct. As such, the nucleic acid constructs are expressed in these cells. A broad range of host cell types may be used in accordance with the present disclosure, e.g., without limitation, bacterial cells, yeast cells, insect cells, mammalian cells or other types of cells.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bactewides spp., Prevotella spp., Clostridium spp., Bifidobacterium spp., or Lactobacillus spp. In some embodiments, the bacterial cells are from Bactewides thetaiotaomicron, Bactewides fragilis, Bactewides distasonis, Bactewides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Entewcoccus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. In some embodiments, the cell is an Escherichia coli cell. In some embodiments, the cell is a Pseudomonas putida cell. “Endogenous” bacterial cells refer to non-pathogenic bacteria that are part of a normal internal ecosystem such as bacterial flora.

In some embodiments, bacterial cells of the present disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, engineered nucleic acid constructs are expressed in mammalian cells. For example, in some embodiments, engineered nucleic acid constructs are expressed in human cells, primate cells (e.g., vero cells), rat cells (e.g., GH3 cells, OC23 cells) or mouse cells {e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1clc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-IOA, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

In some embodiments, the cell is an immune cell. Non-limiting examples of immune cells include B cells, dendritic cells, granulocytes, innate lymphoid cells (ILCs), megakaryocytes, monocytes/macrophages, natural killer (NK) cells, platelets, red blood cells (RBCs), T cells, and thymocytes. In some embodiments, an engineered nucleic acid construct as provided are delivered to B cells.

Cells of the present disclosure, in some embodiments, are modified. A modified cell is a cell that contains an exogenous nucleic acid or a nucleic acid that does not occur in nature (e.g., an engineered nucleic acid encoding a ssDNA-annealing recombinase protein such as Beta recombinase protein). In some embodiments, a modified cell contains a mutation in a genomic nucleic acid. In some embodiments, a modified cell contains an exogenous independently replicating nucleic acid (e.g., an engineered nucleic acid present on an episomal vector). In some embodiments, a modified cell is produced by introducing a foreign or exogenous nucleic acid into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C, et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88). In some embodiments, a cell is modified to express a reporter molecule. In some embodiments, a cell is modified to express an inducible promoter operably linked to a reporter molecule (e.g., a fluorescent protein such as green fluorescent protein (GFP) or other reporter molecule).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing an engineered nucleic acid into the cell in order to produce a genetic change of interest (e.g., via insertion or homologous recombination). In some embodiments, a cell overexpresses genes encoding the subunits of Exo VII of Escherichia coli. Thus, in some embodiments, a cell overexpressed one or more genes encoding XseA and/or XseB of Escherichia coli or homologs thereof.

The cells that may be used in accordance with the present disclosure may have different genetic backgrounds, e.g., unmodified, or comprising different modifications such as a gene deletion. For example, the present disclosure contemplates modified bacterial cells, such as modified E. coli cells. In some embodiments, the modified bacterial cells lack genes encoding RecJ and/or XonA, which are exonucleases. In some embodiments, modified bacterial cells lack one or more other exonucleases, e.g., ExoX nuclease.

The present disclosure also demonstrates, unexpectedly, that, ssDNA mediated recombineering can occur in cells with an active mismatch repair system (e.g., mutS⁺ in FIG. 1A). This is significant because the deactivation of the mismatch repair system (e.g., in a mutS⁻ background) results in elevated background mutation rate. Thus, in some embodiments, the bacterial cell has an intact mismatch repair system but is lacking cellular exonucleases, e.g., RecJ and/or XonA. The genomic editing construct described herein may achieve a high editing efficiency without elevated background mutation rate.

In some embodiments, an engineered nucleic acid construct may be codon-optimized, for example, for expression in mammalian cells (e.g., human cells) or other types of cells. Codon optimization is a technique to maximize the protein expression in living organism by increasing the translational efficiency of gene of interest by transforming a DNA sequence of nucleotides of one species into a DNA sequence of nucleotides of another species. Methods of codon optimization are well-known.

Engineered nucleic acid constructs of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., engineered nucleic acid) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein. Expression of nucleic acids in transiently-transfected and/or stably-transfected cells may be constitutive or inducible. Inducible promoters for use as provided herein are described above.

### Methods

Other aspects of the present disclosure relate to methods that include delivering to cells at least one of the genomic editing constructs as provided herein. Constructs may be delivered by any suitable means, which may depend on the residence and type of cell. For example, if cells are located in vivo within a host organism (e.g., an animal such as a human), engineered nucleic acid constructs may be delivered by injection into the host organism of a composition containing engineered nucleic acid constructs. Constructs may be delivered by a vector, such as a viral vector (e.g., bacteriophage or phagemid). For cells that are not located within a host organism, for example, for cells located ex vivo/in vitro or in an environmental (e.g., outside) setting, engineered nucleic acid constructs may be delivered to cells by electroporation, chemical transfection, fusion with bacterial protoplasts containing recombinant, transduction, conjugation, or microinjection of purified DNA directly into the nucleus of the cells.

Cells to which engineered nucleic acid constructs are delivered typically contain a nucleotide sequence, referred to as a “target sequence,” which is complementary to the targeting sequence of the construct. A target sequence may be located within the genome of the cell, or the target sequence may be located episomally (e.g., on a plasmid) within the cell. In some embodiments, a target sequence is located in an engineered nucleic acid construct. For example, one engineered nucleic acid construct may contain a nucleic acid encoding a targeting sequence that is complementary (or partially complementary) to a target sequence located in another engineered nucleic acid construct. In some embodiments, a cell comprises a reverse transcriptase, (e.g., an endogenous reverse transcriptase). Thus, in some embodiments, methods comprise delivering to such cells engineered nucleic acid constructs that do not encode a reverse transcriptase. In some embodiments, a cell does not comprise a reverse transcriptase. Thus, in some embodiments, methods comprise delivering to such cells engineered nucleic acid constructs that encode a reverse transcriptase. In some embodiments, for example, where a cell does not contain a reverse transcriptase, methods may comprise delivering to cells (a) at least one of the engineered nucleic acid constructs as provided herein that does not encode a reverse transcriptase, and (b) an engineered nucleic acid construct comprising a promoter operably linked to a nucleic acid encoding a reverse transcriptase.

In some embodiments, a cell comprises a ssDNA-annealing recombinase protein (e.g., an endogenous ssDNA-annealing protein such as an endogenous Beta recombinase protein). Thus, in some embodiments, methods comprise delivering to such cells engineered nucleic acid constructs that do not encode a ssDNA-annealing recombinase protein. In some embodiments, a cell does not comprise a ssDNA-annealing recombinase protein. Thus, in some embodiments, methods comprise delivering to such cells engineered nucleic acid constructs that encode a ssDNA-annealing recombinase protein. In some embodiments, for example, where a cell does not contain a ssDNA-annealing recombinase protein, methods may comprise delivering to cells (a) at least one of the engineered nucleic acid constructs as provided herein that does not encode a ssDNA-annealing recombinase protein, and (b) an engineered nucleic acid construct comprising a promoter operably linked to a nucleic acid encoding a single-stranded DNA (ssDNA)-annealing recombinase protein.

In some embodiments, a cell comprises a Cas9 protein, e.g., an endogenous Cas9 protein. Thus, in some embodiments, methods comprise delivering to such cells engineered nucleic acid constructs that do not encode a Cas9 protein. In some embodiments, a cell does not comprise a Cas9 protein (e.g., an active Cas9 nuclease or a dCas9 protein). Thus, in some embodiments, methods comprise delivering to such cells engineered nucleic acid constructs that encode a Cas9 protein or a dCas9 protein. In some embodiments, for example, where a cell does not contain a Cas9 or dCas9 protein, methods may comprise delivering to cells (a) at least one of the engineered nucleic acid constructs as provided herein that does not encode a ssDNA-annealing recombinase protein, and (b) an engineered nucleic acid construct comprising a promoter operably linked to a nucleic acid encoding a Cas9 or dCas9 protein.

Some bacterial cells are resistant to transformation, e.g., having low transformation efficiency. Thus, the present disclosure also contemplates alternative routes of nucleic acid delivering. For example, in some embodiments, the one or more engineered nucleic acid construct may be delivered via transduction. “Transduction” refers to a process by which foreign DNA is introduced into a cell by a virus or viral vector. When the cell is a bacterial cell, transduction is achieved via a bacteriophage (i.e., virus that infects bacteria). Genetic materials to be transferred may be encoded within a phagemid. A phagemid is a plasmid that contains an fl origin of replication from an fl phage. A phagemid may be replicated as a plasmid, and also be packaged as single stranded DNA in viral particles. For example, the genomic editing constructs described herein may be encoded within a phagemid and packaged into a phage particle in a packaging strain (Chasteen et al., Nucleic Acids Research, 34, e145 (2006), incorporated herein by reference). The phage particle may then be isolated and enriched for delivering into a desired cell.

In some embodiments, the genomic editing construct described herein may be delivered to a desired cell via conjugation. “Conjugation” refers to the transfer of genetic material between bacterial cells by direct cell-to-cell contact or by a bridge-like connection between two cells. The mechanism underlying the conjugation process is horizontal gene transfer. During conjugation, a donor cell provides a conjugative or mobilizable genetic element that is most often a plasmid or transposon. In some embodiments, the genomic editing constructs of the present disclosure may be constructed such that it may be maintained in a conjugation donor strain (e.g., a DAP-auxothrophic MFDpir strain), e.g., be constructed in a plasmid containing an origin of transfer (e.g., an oriT). The conjugation donor strain may then be contacted with the cell to be modified, thereby transferring the genomic editing construct via conjugation.

In some embodiments, a promoter (e.g., an inducible promoter) is operably linked to the nucleotide sequence encoding the genetic elements of the genome-editing construct described herein. As such, the expression of these genetic elements may be activated via a signal, e.g., a chemical or non-chemical. Thus, in some embodiments, methods comprise exposing cells that contain engineered nucleic acid constructs as provided herein to at least one signal that regulates transcription of at least one nucleic acid of a construct. A signal that regulates transcription of nucleic acid may be a signal (e.g., chemical or non-chemical) that activates, inactivates or otherwise modulates transcription of a nucleic acid. For transcription of a nucleic acid of an engineered nucleic acid construct of the present disclosure to be regulated, conditions under which cells are exposed should permit transcription. Such conditions will depend on the cells and the genetic elements used to construct the engineered nucleic acid constructs (e.g., exposing cells to signals (e.g., chemical or non-chemical conditions) known to regulate transcription of particular inducible promoters).

In some embodiments, a cell that contains engineered nucleic acid constructs is exposed more than once to a signal that regulates transcription of a nucleic acid of an engineered nucleic acid construct as provided herein. For example, a cell may be exposed to a signal 2, 3, 4, 5, 6, 7, 8, 9, 10 or more times. The cell exposure may occur over the period of minutes (e.g., 5, 10, 15, 20, 25, 30, 35, 40, 45, 50 or 55 minutes), hours (e.g., 1, 2, 3, 4, 5, 6, 7, 8, 9 10, 11, 12, 13, 14, 15, 16, 17, 18, 19, 20, 21, 22 or 23 hours), days (e.g., 2, 3, 4, 5 or 6 days), weeks (e.g., 1, 2, 3 or 4 weeks), or months (1, 2, 3, 4, 5, 6, 7, 8, 9, 10, 11 or 12 months), or for a shorter or longer duration. Cell exposure may be at regular intervals or intermittently.

In some embodiments, a signal that activates transcription is an endogenous signal, meaning that the signal is generated from within the cell or by the cell. For example, cell exposure to certain environmental conditions may cause the cell to produce, intracellularly or extracellular, a chemical or non-chemical signal that activates transcription of a nucleic acid of an engineered nucleic acid construct of the present disclosure.

In some embodiments, cells that contain one or more engineered nucleic acid construct of the present disclosure are permitted to express the constructs (e.g., incubated at conditions suitable for cell expression) for a prolonged period of time (e.g., at least 2 days, at least 3 days, at least 4 days, at least 5 days, at least 6 days, at least 7 days, at least 8 days, at least 9 days, at least 10 days, or more).

In some embodiments, cells that express the Exo VII complex and contain one or more engineered nucleic acid construct of the present disclosure are permitted to express the constructs for a shortened period of time (e.g., less than 2 days, less than 1 day, or less than 12 hours).

### Applications

Recently, different technologies for record of molecular events in DNA of living cells are described. Memory recording using site-specific recombinases and CRISPR spacer acquisition require cis-acting elements and recording is confined within a predefined sequence. The engineered constructs as provided herein do not require any cis-encoded sequence on the target and as such opens up the entire genomic repertoire for high-efficiency genomic editing and single-cell memory applications. Furthermore, unlike high-efficiency genomic editing strategies that rely on counter selection by CRISPR nuclease, engineered constructs as provided herein enable active and dynamic modification of bacterial genomes without requirement to introduce double-stranded DNA break and avoids associated cytotoxicity, chromosomal rearrangements and unwanted genome-wide sweeps, which are especially important in cases where precision modifications are desired or where cellular fitness is important (e.g., in the context of editing bacterial communities or evolution experiments).

The present disclosure offers a framework for dynamic engineering of bacterial genomes with high efficiency and precision and provides methods for recombineering in previously inaccessible organisms having limited transformation efficiency. By linking high-efficiency genomic editing with cellular cues, the CRISPRi/SCRIBE system of the present disclosure enables in situ engineering of bacterial genome within bacterial communities, continuous in vivo evolution of single-gene (e.g., protein function) or multi-gene (e.g., metabolic networks) traits, and directed evolution of specific segments of genomes in response to cellular and environmental cues.

In some embodiments, methods and compositions of the present disclosure may be used for high efficiency genomic editing in live cells of any genetic background, and in any context, e.g., a wild type bacterial cell within a bacterial community. In some embodiments, the methods and compositions of the present disclosure may be used to specifically modify the genome of a bacterial cell within a bacterial community in situ, without affecting other bacterial cells in the community. A “bacterial community,” as used herein, refers to a collection of bacteria of one or more species at a certain site, e.g., the human gastrointestinal tract. Different bacterial cells in a bacterial community may possess their unique genomic sequences and phenotypical traits, e.g., resistance to a certain antibiotic such as ampicillin. As such, a sub-population of the bacterial community may be modified using the genomic editing constructs and methods described herein. For example, to specifically modify a bacterial cell, e.g., a bacterial cell that is resistant to an antibiotic, the genomic editing construct may be designed so that the nucleotide sequence encoding the msdDNA is modified to contain a targeting sequence, e.g., a target sequence that targets the antibiotic resistance gene, wherein the target gene, e.g., the antibiotic resistance gene, comprises a nucleotide sequence that is complementary to the targeting sequence. In some embodiments, the genomic editing construct may be delivered to the bacterial community, e.g., via transduction or conjugation. Upon delivery into the bacterial cells in the bacterial community, the bacterial cell that contain the target gene, e.g., the antibiotic resistance gene, is modified and the antibiotic resistance gene is inactivated. It is to be understood that the genomic editing construct may also enter cells that do not contain the target gene. However, due to the absence of the target sequence, cells that do not contain the target gene will not be modified. Further, the efficiency of editing may be augmented by designing the genomic editing construct to encode gRNAs that target the cellular exonucleases, e.g., RecJ and/or XonA. Thus, the compositions and methods described herein, enable in situ modification of a bacterial cell within a bacterial community with high specificity and efficiency. Furthermore, such methods neutralize undesirable cells, e.g., an antibiotic resistance bacterial cell in a human gastrointestinal tract, without killing the cell, thus avoiding the negative effect that may result from a completely removal, e.g., killing, of a type of bacterial cell from a bacterial community. It is to be understood that the example is for illustration purpose only and is not meant to be limiting. The compositions and methods described herein may be used for any targeted modification of a bacterial cell in a bacterial community, for any desired purpose.

In some embodiments, the compositions and methods described herein may be used to functionalize a cell, e.g., to activate a naturally silent gene in the cell. As such, the genomic editing construct described herein may be designed so that the msdDNA contains a targeting sequence that targets the naturally silent gene, e.g., in a transcriptional suppressor binding site, to thereby activate the gene. In some embodiments, the targeting sequence may target a repressor gene of the naturally silent gene to deactivate the repressor gene. In some embodiments, the targeting sequence may target the promoter or ribosome binding site of the naturally silent gene, to create a stronger promoter or ribosome binding site, to thereby enhance the expression of the gene. Such naturally silent genes may be, without limitation, an enzyme, a transcriptional regulator, genes that encode small metabolites, or antibiotic resistance genes.

In some embodiments, the compositions and methods described herein may be used for evolution of a living cell or a biological molecule, e.g., a protein or a nucleic acid. Living cells are capable of sense environmental cues and in response, optimize their fitness in a given environment. Such response vary depending on the time-scale of the environmental cues. For example, in some embodiments, short-term cues are responded by regulation of transcriptional and translational programs, while cues that last within evolutionary time-scales are responded by permanent genetic alterations, e.g., mutations. Accumulation of these adaptive genetic alteration over the evolutionary time-scales leads to increase of fitness of the organism in a given environment, which in turn results in the dominance of the associated genotype. Such evolutionary process may be harnessed in a laboratory, termed “directed evolution,” in the form of iterative cycles of diversity generation and screening (Esvelt et al., Nature 472, 499-503 (2011), incorporated herein by reference). Using directed evolution, an organism, or a biological molecule, e.g., a protein or a nucleic acid, may be evolved toward a user-defined goal. To apply the genomic editing methods described herein to achieve directed evolution, the genomic editing constructs may be linked to a continuous selection/screening setup. Example 5 of the present disclosure demonstrates the continuous evolution of the P_{lac} locus in bacterial cells using the compositions and methods described herein.

In some embodiments, the evolution rate may be accelerated by counter selection against the undesired allele by designing the nucleotide sequence encoding the gRNA in the genomic editing constructs to target the wild type allele and providing an active Cas9 nuclease to introduce double-stranded DNA breaks in the wild type allele and cause cell death. In some embodiments, genomic editing efficiency improved by designing the nucleotide sequence encoding the gRNA in the genomic editing constructs to target cellular exonucleases, e.g., RecJ and/or XonA, and providing a catalytically inactive Cas9 (dCas9), to thereby downregulate the cellular exonucleases that negatively affect the genomic editing efficiency.

In some embodiments, the genomic editing compositions and methods of the present disclosure may be used to diversify a desired genomic locus. To diversify a genomic locus, the genomic editing construct of the present disclosure may be engineered to specifically increase the mutation rate at the desired genomic loci, without increasing the global mutation rate. For example, in some embodiments, diversity may be introduced into the targeting sequence in the msdDNA during its generation, via error-prone RNA polymerase and/or error-prone reverse transcriptase (Brakman et al., Chembiochem. 2001 Mar. 2; 2(3):212-9, Bebenek et al., The Journal of Biological Chemistry, Vol. 268, No. 14, Issue of May 15, pp. 10324-10334, 1993, and Pulsinelli et al., PNAS, Vol. 91, pp. 9490-9494, September 1994, incorporated herein by reference). In some embodiments, DNA modifying enzymes that modify RNA molecules or ssDNA molecules may be used in conjunction with the genomic editing construct of the present disclosure. Such DNA modifying enzymes introduce site-specification mutations into the msdRNA or the msdDNA after they are made. Suitable DNA modifying enzyme that may be used in accordance with the present disclosure include, without limitation, cytosine deaminases, e.g., AID (Bransteitter et al., PNAS, 100 (7): 4102-7 (2003), incorporated herein by reference) and adenosine deaminases, e.g., ADA (Keegan et al., Genome Biology 2004 5:209, incorporated herein by reference). In some embodiments, the repair machinery of the cell may be conditionally suppressed to increase the mutation rate. For example, the genomic editing construct may be engineered to express a gRNA that targets the MMR system or the uracil-DNA glycosylase in the cell. Such targeted diversification methods described herein may be used in different cells, e.g., a bacterial cell, or a B cell for the diversification of antibodies. The examples provided herein are not meant to be limiting.

In some embodiments, an evolvable cell may be constructed, e.g., an evolvable bacterial cell. In some embodiments, the evolvable cell may be engineered to express neutralizing antibodies on their surface. The genomic editing construct may be coupled with a signaling circuit, which signals the cell to express the msdDNA to modify a gene locus, e.g., a nucleotide sequence encoding the neutralizing antibody. In some embodiments, such signal may be triggered by the binding of a pathogen to the antibody on the cell surface. One of the many advantages of the genomic editing construct described herein is that it can be easily repurposed to targeted and re-target a desired sequence. This method would lead to rapid diversification of the antibody locus, thus expanding the antibody repertoire and enabling the fact evolving of antibodies in response to the evolving pathogen. Further, the targeted diversification process described herein may be useful in other applications such as engineering phage host range to adapt gene circuits. In summary, the genomic editing compositions and methods described herein open up a broad range of new capabilities for, e.g., biomedical research, synthetic biology, highly efficient directed evolution, targeted diversification, and in situ genomic editing of cells of any genetic background in any context.

Connectome Mapping.

In some embodiments, methods and compositions described herein may be used to map a cellular connectome. A donor barcode (d-barcode) may be transferred to a recipient cell, where it is written next to a unique barcode on the recipient genome (r-barcode). By sequencing the adjacent barcodes on the recipient genome, the connectivity matrix between the donors and recipients can be deduced.

A “donor cell” is a cell that transfers a unique barcode to a recipient cell. A donor cell may be a bacterial cell or a eukaryotic cell. In some embodiments, the donor cell is a presynaptic neural cell. A “recipient cell” is a cell that receives a barcode from the donor cell. A recipient cell may be a bacterial cell or a eukaryotic cell. In some embodiments, the recipient cell is a postsynaptic neural cell.

A “d-barcode” is a nucleotide sequence that uniquely barcodes the donor cell (the identity of the donor cell may be determined based on the d-barcode composition). In some embodiments, a d-barcode is encoded on a mobile genetic element, for example, which can then be transferred from the donor cell to the recipient cell. A “r-barcode” is a nucleotide sequence that uniquely barcodes the recipient cell and generally should not be mobilized. In some embodiments it is located on the recipient genome.

Both d-barcodes and r-barcodes may be synthesized in vitro, for example, and introduced to the donor or recipient cell, respectively, by transformation or transfection (or other delivery method). In some embodiments, a barcode may be introduced using a site-specific nuclease to induce a double-stranded DNA (dsDNA) break, resulting in error-prone non-homologous end joining (NHEJ) and leaving a scar that may be used as a barcode. In some embodiments, the site-specific nuclease is CRISPR-Cas9. The d-barcode may then be transferred to the recipient cell using, for example, a mobilizable delivery vehicle. In some embodiments, the delivery vehicle may be a virus or outer membrane vesicle. In other embodiments, the nucleotide conveyance between the two cells may be accomplished by direct cell-to-cell transfer.

In some embodiments, multiple d-barcodes may be transferred and written next to the recipient barcode, enabling the recordation of multiple interactions within a single cell. Once the d-barcode is transferred to the recipient cell, it is written next to the recipient barcode (in cis). In some embodiments, this is accomplished by genome editing techniques permitting efficient homologous recombination. For example, Synthetic Cellular Recorders Integrating Biological Events (SCRIBE) or other genome editing techniques that rely on site-specific nucleases to increase homologous recombination efficiency or techniques that enable efficient genome integration of the mobile genetic element may be used. In some embodiments, the site-specific nuclease may be CRISPR/Cas9 or NgAgo. In other embodiments, transposable elements may be used to achieve genome integration. The adjacent barcodes on the recipient cells may then be PCR amplified and read by high-throughput sequencing. The connectivity matrix may then be deduced by identifying d-barcodes and r-barcodes that are linked in the sequencing reads.

In some embodiments, methods and compositions of the present disclosure may be used for mapping transient interactions with dynamic genome engineering and DNA sequencing. The method may include, for example, the conditional transfer of a unique barcode from a prey-plasmid (p-barcode) next to a unique code on a bait plasmid (b-barcode). The writing only occurs if the two proteins, prey and bait, interact. Protein-protein interactions are one form of transient interaction contemplated herein. The prey and bait proteins may be expressed from plasmids, for example, harboring unique DNA barcodes. The conditional writing system writes the p-barcode next to the b-barcode upon the successful interaction between the bait and prey proteins. In some embodiments, two halves of a split protein are fused to bait and prey proteins. The split protein may be, but is not limited to, a split transcription factor.

In some embodiments, the split protein is GAL4. When bait and prey proteins successfully interact, a functional GAL4 is formed, leading to expression of a gRNA that, in the presence of Cas9, introduces a dsDNA break on the bait plasmid, initiating homologous recombination and writing of the p-barcode on the bait plasmid next to the b-barcode.

In some embodiments, the split protein is Cas9. The bait and prey proteins may be fused to halves of a Cas9, for example, so that if the bait and prey proteins interact a functional Cas9 is formed and the p-barcode is written next to the r-barcode by sequence homology. The adjacent barcodes on the bait plasmid are then PCR-amplified and read by high-throughput sequencing.

Interactions may be deduced by identifying p-barcodes and b-barcodes that are linked to the sequencing reads. Other types of interactions in addition to protein-protein interactions can be recorded in analogous ways.

### Compositions and Kits

Other aspects of the present disclosure also provide compositions and kits containing the engineered nucleic acid constructs and cells described herein. Such compositions and kits may be designed for any of the methods and applications described herein.

The compositions and kits described herein may include one or more engineered nucleic acid constructs to perform the genomic editing methods described herein and optionally instructions of uses. Specifically, such a composition or kit may include one or more agents described herein (for example, a bacterial strain that is competent in conjugation), along with instructions describing the intended application and the proper use of these agents. Compositions and kits (e.g., for research purposes) may contain the components in appropriate concentrations or quantities for running various experiments.

Any of the compositions or kits described herein may further comprise components needed for performing the assay methods. For example, they may contain components for use in detecting a signal released from the labeling agent, directly or indirectly. In some examples, the detection step of the assay methods involves enzyme reaction, the composition or kit may further contain the enzyme and a suitable substrate.

Each component of the compositions and kits, where applicable, may be provided in liquid form (e.g., in solution), or in solid form, (e.g., a dry powder). In certain cases, some of the components may be constitutable or otherwise processable (e.g., to an active form), for example, by the addition of a suitable solvent or other species (for example, water or certain organic solvents), which may or may not be provided with the kit.

In some embodiments, the compositions and kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which can also reflects approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention. Additionally, the kits may include other components depending on the specific application, as described herein.

The compositions and kits may contain any one or more of the components described herein in one or more containers. The components may be prepared sterilely, packaged in syringe and shipped refrigerated. Alternatively it may be housed in a vial or other container for storage. A second container may have other components prepared sterilely. Alternatively the kits may include the active agents premixed and shipped in a vial, tube, or other container.

The compositions and kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The compositions and kits may be sterilized after the accessories are added, thereby allowing the individual accessories in the container to be otherwise unwrapped. Compositions and kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following Examples demonstrate transient non-transcriptional biological information/events can be converted into DNA memory as well as how to map the spatial configuration/connectome of cells within a bacterial colony.

### Example 1: Recombineering in Cells Having an Activated MMR System

The efficiency of oligo-mediated recombineering is limited by the cellular mismatch repair system, but deactivating MMR leads to ˜two orders of magnitude increase in the recombination efficiency of synthetic oligos (1). Thus, deactivating a bacterial cell's MMR system, for example, by knocking out mutS, was thought to be necessary for achieving efficient genome editing when recombineering with synthetic oligonucleotides. ΔmutS strains, which have a deactivated mismatch repair system, have elevated background mutation rates. The data provided in this Example shows, unexpectedly, that efficient recombineering using the engineered constructs of the present disclosure can be performed in a bacterial strain having an active mismatch repair system.

Using a KanR reversion assay (in which premature stop codons within a genomic KanR cassette are reverted back to the wild-type sequence by intracellularly expressed ssDNAs (13)), the efficiency of recombination in different knockout backgrounds was measured. As, shown in FIG. 1A, deactivating the MMR system (ΔmutS background) resulted in a modest increase in the efficiency of recombination.

By contrast, knocking out cellular ssDNA-specific exonucleases (recJ and xonA, which encode 5′-specific and 3′-specific ssDNA exonucleases, respectively), which could limit the availability of ssDNA inside the cell, significantly increased the efficiency of recombination, suggesting that the performance of the engineered constructs is limited by the availability of intracellular ssDNAs. Surprisingly, there was a synergistic increase in the efficiency of recombination in the ΔrecJ ΔxonA background, resulting in recombination frequencies comparable with highest reported recombineering efficiency for oligo-mediated recombineering in a ΔmutS background (3, 14).

Knocking out cellular exonucleases also increased the background recombination frequency in the absence of SCRIBE induction (FIG. 1A). To investigate this result, the recombinant frequencies in the presence and absence of reverse transcriptase (RT) activity were measured. As shown in FIG. 1A, elevated recombination was observed even in the absence of the reverse transcriptase (RT) activity. Nonetheless, in all of the conditions tested, presence of RT activity resulted in about two orders of magnitude increase in the frequency of recombinants, demonstrating that the recombination efficiency was improved when ssDNA is expressed. This intracellular ssDNA pool is naturally degraded by cellular exonucleases, thus limiting the efficiency of recombination in the wild-type (WT) background. When cellular exonucleases are knocked out, the retron-encoded ssDNA, as well as the template double-stranded DNA, can contribute to the intracellular ssDNA pool and increase the recombination efficiency (FIG. 1B). Beta recombinase protects the intracellular oligonucleotide pool from cellular exonucleases and facilitate recombination between the ssDNAs and their corresponding genomic target loci (FIG. 1B).

Knocking out xseA, one of the two subunits of ExoVII, slightly reduced the recombination efficiency of the engineered constructs. ExoVII is a ssDNA-specific exonuclease that converts large ssDNA substrates into smaller oligonucleotides (18). This nuclease is responsible for removal of phosphorothioated nucleotides from flanking ends of recombineering oligos (19) and also for removal of the msr moiety from msdDNA of RNA-less retrons (20). These observations suggest that ExoVII, among other cellular factors, is involved in generating recombinogenic ssDNA intermediates. recBCD-mediated processing of double-stranded breaks may be another possible source of recombinogenic intracellular ssDNA pool (21).

To demonstrate that high-efficiency genome modification can be performed in a wild-type (WT) background (having an active MMR system), identified exonucleases were knocked down using CRISPRi (22). Two gRNAs targeting xonA and recJ as well as dCas9 under control of aTc-inducible promoters were cloned in to a CRISPRi-nuc2gRNA plasmid (FIG. 1C), which was then co-transformed along with the IPTG-inducible SCRIBE plasmid into DH5α PRO kanR_{OFF} reporter strain. Induction of either SCRIBE or CRISPRi systems resulted in a modest increase in the recombination efficiency. Co-induction resulted in 4-logs increase in the recombination efficiency. Recombinants were not detected in cells that were transformed with SCRIBE(NS) plasmid and frequency of recombinants was significantly lower when cells were transformed with a CRISPRi system lacking the gRNAs. These results further confirmed that the presence of recombinogenic oligonucleotides is limited by cellular exonucleases and indicated that high-efficiency genomic editing can be achieved with the engineered constructs of the present disclosure in the WT strain, and is not limited to a specific genetic background. The high-efficiency inducible DNA writing could be coupled to natural or synthetic regulatory circuits and combined with logic operations (such as the AND gate shown in this example) for single-cell computation-and-memory applications, for example.

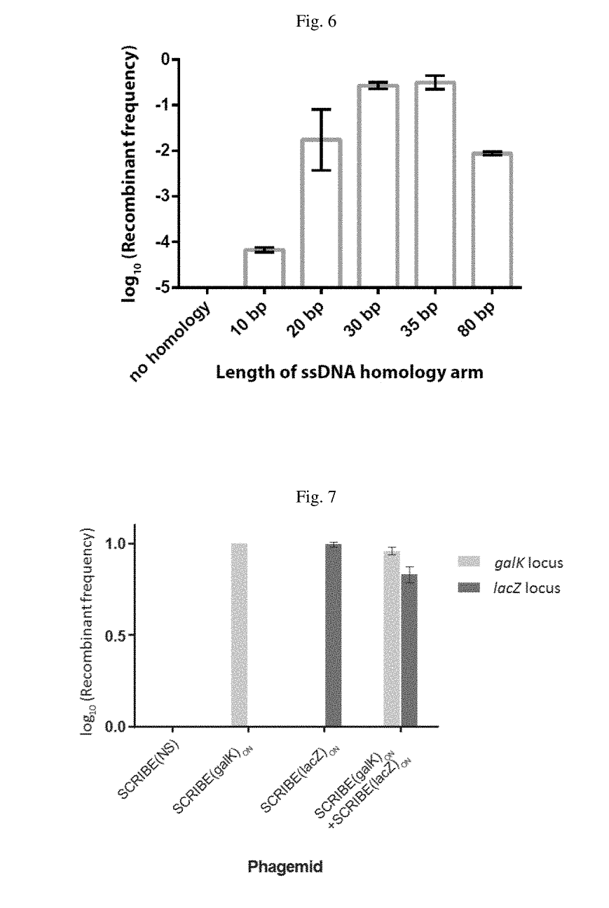

Despite an increased editing efficiency in the ΔrecJ ΔxonA background, full allele conversion was not observed in the kanR reversion assay within 10 generations; only ˜10% of cells became recombinant after 24 hours (corresponding to ˜10 generations) of induction (FIG. 1A). The recombination efficiency was increased to 36% when a strong ribosome binding site (RBS) was used to overexpress beta (FIG. 6). It is possible that in the PRO strain (which overexpresses tetR and lacI), P_{lacO} promoter is subjected to all-or-non induction, and even at high concentrations of the inducers, a fraction of cells did not express SCRIBE at high levels, thus lowering the maximal editing efficiency.

Furthermore, since beta-mediated recombineering is a replication-dependent process (17, 24), the recombination efficiency is increased if cells are allowed to grow for more generations (e.g., by spatially separating and growing them on plates). To overcome these limitations, a screening assay was developed based on reversion of galK negative cells (galK_{OFF} cells containing two premature stop codons within the middle of galK gene) to galK positive cells (galK_{ON}) by SCRIBE. Two stop codons were introduced into the galK ORF of MG1655 ΔrecJ ΔxonA strain (galK_{OFF} reporter strain). This reporter was converted from galK− to galK+ upon transformation of the SCRIBE(galK)_{ON} (SCRIBE plasmid encoding ssDNA homologous to the WT galK), and the galK+ bacterial cells were screened on screenable MacConkey+Gal plates. As shown in FIG. 1D, more than 99% of galK− (white) cells transformed with the SCRIBE(galK)_{ON} plasmid were converted to galactose fermenting galK+ (pink) colonies. Pink colonies were not detected when cells were transformed with a non-specific SCRIBE (SCRIBE(NS)) plasmid. Sanger sequencing of PCR amplicons of galK locus obtained from the pink colonies indicated the conversion of galK_{OFF} allele to galK_{ON} to the extent that the presence of galK_{OFF} allele was below the limit of detection.

### Example 2: Counter-Selection Against Undesired Alleles

The enrichment of a beneficial allele within a bacterial population directly correlates with its fitness. In the absence of a selective advantage, it may take many generations for a neutral allele to enrich within a population. The rate of this gene conversion process may be increased by putting a selective pressure against the wild-type (WT) allele at the nucleotide level. As shown in this Example, the engineered constructs of the present disclosure were used to edit a particular locus in the genome of a bacterial population, thereby introducing a modified (e.g., beneficial) allele. The CRISPR/Cas9 system is then used to counterselect against the corresponding WT allele. Surprisingly, this method enabled highly efficient modification of a bacterial genome in a particular population in a short period of time—after 12 hours induction of Cas9 nuclease—to the extent that WT allele in the population becomes undetectable (e.g., by ILLUMINA® sequencing).

An aTc-inducible gRNA against the galK_{OFF} allele was placed into the SCRIBE(galK)_{ON} plasmid and transformed into the galK_{OFF} reporter cells expressing aTc-inducible Cas9, or dCas9 (as negative control) plasmids. Single colonies of transformants were grown for 12 hours with or without aTc. galK allele frequencies within the population were measured by ILLUMINA® sequencing before and after induction by aTc. As shown in FIG. 1E, mutant alleles enriched in all the cultures over time, indicating that genomic editing via SCRIBE is a replication/time-dependent process. Upon induction with aTc, the mutant alleles were enriched faster in cells expressing Cas9 in comparison to cells expressing dCas9, approaching 100% editing efficiency within 12 hours after induction. These results demonstrate that genomic editing with the SCRIBE system can be combined with counter-selection via the CRISPR/Cas9 system to accelerate enrichment of modified (e.g., beneficial) alleles.

### Example 3: High-Efficiency Genomic Editing in MG1655 E. coli

Oligo-mediated recombineering is a powerful technique to introduce desired modifications into a bacterial genome. Nonetheless, since synthetic oligonucleotides are introduced to the target cells transiently (via electroporation) and intracellular oligonucleotides have a short half-life, the theoretical editing efficiency of oligo-mediated recombineering is limited to 25%, while the practical editing efficiency is often limited to a few percent (3, 14). Furthermore, the technique relies on a high-efficiency transformation protocol and is only applicable to conditions/organisms where high efficiency transformation is possible. In addition, to achieve high efficiencies of genomic editing, modification of the host by knocking down the MMR system is often required, which in turn elevates the global mutation rate and leads to off-target mutations (25). The engineered constructs of the present disclosure provide a persistent source of recombinogenic oligos intracellularly over many generations, and can be introduced to cells even with low efficiency delivery methods, thus bypassing both of the above-mentioned limitations. Furthermore, expression of ssDNAs that harbor mismatches in the stem region could to some extent titrate out MutS (15), thus providing a built in add-on to conditionally knockdown MMR system and increase genomic editing efficiency.

To demonstrate this, the SCRIBE system and the CRISPRi system described in Example 1 were placed into a single synthetic operon (as shown in FIG. 2A), cloned it into a plasmid and this plasmid was transformed into a MG1655 galK_{OFF} reporter strain. Cells were chemically transformed with either SCRIBE(galK)_{ON} or SCRIBE(NS), outgrown in LB for an hour, serially diluted and plated on MacConkey+Gal+antibiotic plates. More than 99% of cells transformed with the SCRIBE(galK)_{ON} plasmid formed pink colonies on these plates, indicating successful writing on the galK locus. Pink colonies were not detected in the samples transformed with SCRIBE(NS) plasmid. Since beta-mediated recombineering is a replication-dependent process (17, 24), the conversion of galK- to galK+ phenotype happens over the course of growth of the colonies, and a single pink colony observed on a transformation plate may contain a heterogeneous population of galK- and galK+ cells. The frequency of these alleles within single colonies 24 hours after transformation (corresponding to ˜31 generations) was assessed by PCR amplification of galK locus followed by ILLUMINA® sequencing. As shown in FIG. 2B, more than 75% of alleles in the singles colonies were mutated within 24 hours, while the mutant alleles were below the detection limit in the negative control.

### Example 4: Genomic Editing of Bacteria in Synthetic Bacterial Communities

Oligo-mediated recombineering is only limited to organisms and conditions where transformation with high efficiency (usually through electroporation) is achievable. On the other hand, the engineered constructs as provided herein can be delivered to cells via alternative delivery methods such as conjugation and transduction. SCRIBE plasmid can be encoded within a phagemid, packaged into phage particles and specifically delivered to desired cells within a bacterial community. To demonstrate this, SCRIBE phagemids were packaged (harboring M13 phage origin of replication) into M13 phage particles using a packaging strain (26) and the phagemid particles were concentrated and introduced it to the galK_{OFF} reporter strain harboring F plasmid (which encodes the receptor for M13 phage). As shown in FIG. 2A, more than 99% of reporter cells that were transduced with M13 phage particles formed pink colonies on the MacConkey+Gal plates. Further analysis of the few white colonies (less than 0.5%) found on these plates indicated that they harbored SCRIBE plasmids with deletions, likely generated during packaging of the phagemids (all sequenced plasmids had deletions at the exact nicking site of the M13 origin of replication. No pink colonies were detected on the negative control where cells were transformed with SCRIBE(NS) phagemid particles. Further, SCRIBE phage particles were shown to be able to target and edit specific cells within a synthetic bacterial community. First, spontaneous Streptomycin resistant ((St^{R}) mutants of the MG1655 F⁺ galK_{OFF} reporter strain was obtained. This reporter strain was then co-cultured with an undefined bacterial community obtained from mouse stool. The purified SCRIBE(galK)_{ON}-encoding phage particles were introduced into this synthetic community. As shown in FIG. 2C, more than 99% of the transductants formed pink colonies on the indicator plates, demonstrating successful editing of the reporter cells within these community. Pink colonies were not observed in the negative control where a non-specific SCRIBE phagemid was delivered to the community.

Similar to transduction, conjugation is another form of horizontal gene transfer in natural bacterial communities. The engineered constructs as provided herein can be delivered by conjugation to edit cells within a bacterial community. An origin of transfer of RP4 plasmid (oriT) was encoded into the SCRIBE(galK)_{ON} plasmid and the plasmid was introduced into DAP-auxothrophic MFDpir cells to produce a donor strain and showed that these cells can conjugate the SCRIBE(galK)_{ON} plasmid into the recipient cells (MG1655 Sp^{R} galK_{OFF}). More than 99% transconjugants formed pink colonies on MacConkey+gal+antibiotic plates. Pink colonies were not obtained in cells that had been conjugated with a non-specific SCRIBE plasmid. It was further demonstrated that conjugation can be performed in the context of bacterial synthetic community by conjugating SCRIBE(galK)_{ON} plasmid to the abovementioned synthetic community. Again, more than 99% of transconjugants that received the SCRIBE(galK)_{ON} plasmid formed pink colonies on the screening plates and pink colonies were not detected in cells conjugated with a non-specific SCRIBE plasmid (FIGS. 2C and 2D). These results demonstrate that different delivery methods can be used to successfully deliver the engineered constructs of the present disclosure into bacterial communities, thus opening up new avenues for performing genomic editing in situ for different applications. Unlike recent genomic editing strategies enabled by counter-selection using site-specific nucleases, the CRISPRi-SCRIBE platform provided herein does not rely on double-stranded breaks and its associated cytotoxicity (27), thus minimizing the associated fitness costs. This property could be especially important for genomic editing in situ in context of bacterial communities, where slight fitness effects could be extremely deleterious.

It was further shown that conjugation, a common strategy for horizontal gene transfer in natural bacterial communities, can be used to deliver the χHiSCRIBE plasmid for genome editing within bacterial communities (FIG. 2J). However, the efficiency of plasmid delivery by conjugation was lower than transduction (FIG. 2K). These results demonstrate that diverse strategies can be used to deliver HiSCRIBE constructs into complex bacterial communities with the potential for in situ genome editing applications.

To facilitate the delivery of HiSCRIBE for DNA writing in non-modified hosts, the HiSCRIBE and CRISPRi systems were placed into a single synthetic operon (referred to as χHiSCRIBE operon as shown in FIG. 2I), cloned it into a high-copy number plasmid, and assessed its performance in the WT MG1655 galK_{OFF} reporter strain, which harbors two stop codons within the galK locus. Cells were chemically transformed with either χHiSCRIBE(galK)_{ON} or χHiSCRIBE(NS), which expressed a galK_{ON} ssDNA or a non-specific ssDNA, respectively. The cells were recovered in LB for an hour, then plated on MacConkey+gal+antibiotic plates to select for χHiSCRIBE plasmid delivery and screen for galK_{OFF} to galK_{ON} editing. More than 99% of cells transformed with the χHiSCRIBE(galK)_{ON} plasmid formed pink colonies on these plates, indicating successful writing in the galK locus in all cells that received this plasmid (FIG. 2I). No pink colonies were detected in the samples transformed with the χHiSCRIBE(NS) plasmid. The frequency of editing within individual colonies was assessed by PCR amplification of galK locus followed by high-throughput sequencing at 24 hours after transformation, as well as after a re-streaking step as described before (FIG. 2I).

Similar to transduction, conjugation is a common strategy for horizontal gene transfer in natural bacterial communities. In addition to using transduction for delivering χHiSCRIBE plasmids, it was tested whether conjugation can be used to deliver and edit cells within a complex bacterial community. The origin of transfer from RP4 (oriT) was encoded into the χHiSCRIBE(galK)ON plasmid and then introduced this plasmid into MFDpirPRO cells (that harbor RP4 conjugation machinery) to produce a donor strain. It was shown that these cells could conjugate the χHiSCRIBE(galK)_{ON} plasmid into recipient cells (MG1655 StrR galK_{OFF}). More than 99% of transconjugants formed pink colonies on MacConkey+gal+antibiotic plates (FIG. 2J), while no pink colonies were obtained in recipients that had been conjugated with the non-specific χHiSCRIBE(NS) plasmid. The χHiSCRIBE(galK)_{ON} plasmid was then conjugated into a stool-derived bacterial community containing MG1655 StrR galKOFF, analogously to the transduction experiments (FIG. 2C). More than 99% of transconjugants that received the χHiSCRIBE(galK)ON plasmid formed pink colonies on the screening plates and no pink colonies were detected in cells conjugated with the non-specific χHiSCRIBE(NS) plasmid (FIG. 2J). However, the efficiency of delivery via conjugation was significantly lower than phagemid transduction (FIG. 2K). It was thought that more specific transduction delivery mechanisms are better suited for editing specific species within a community, while more generalized (albeit less efficient) conjugation delivery mechanism is better suited for situations where editing a larger subpopulation of bacteria in the community are desired.

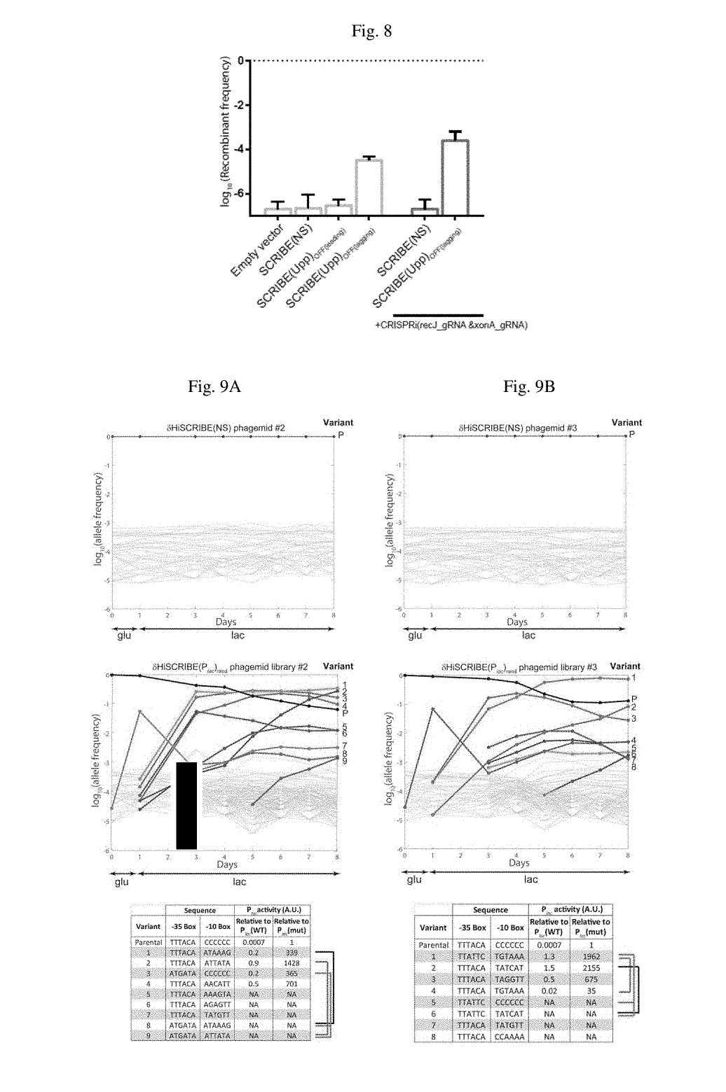

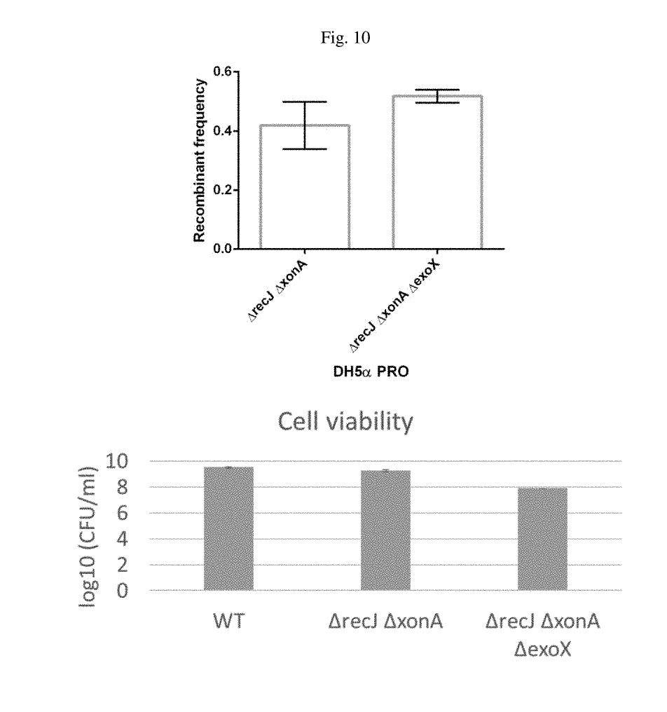

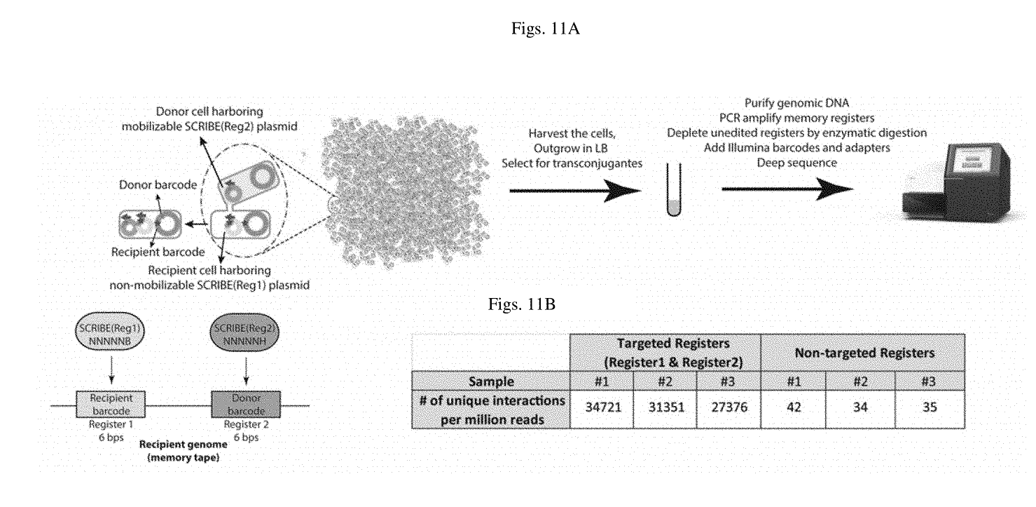

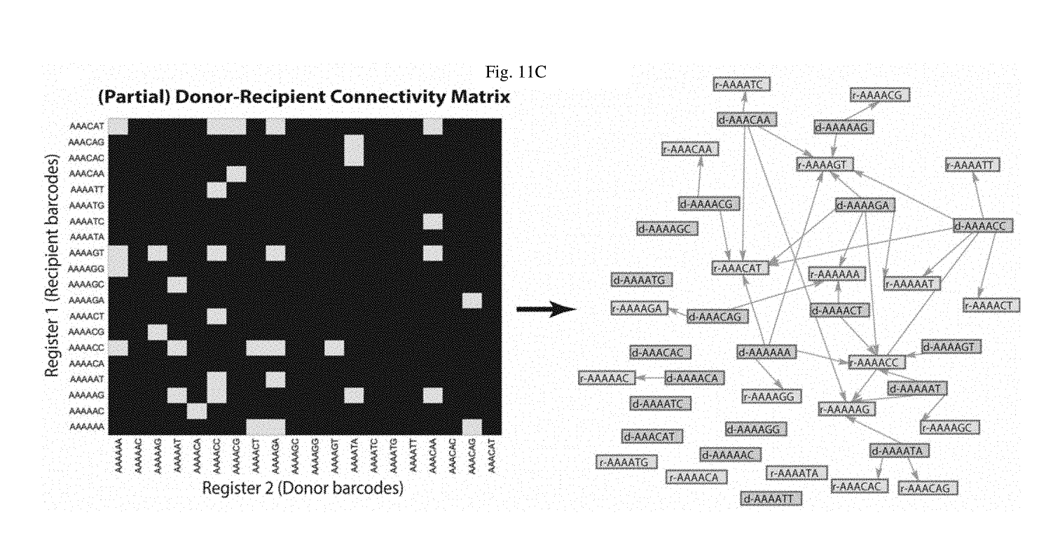

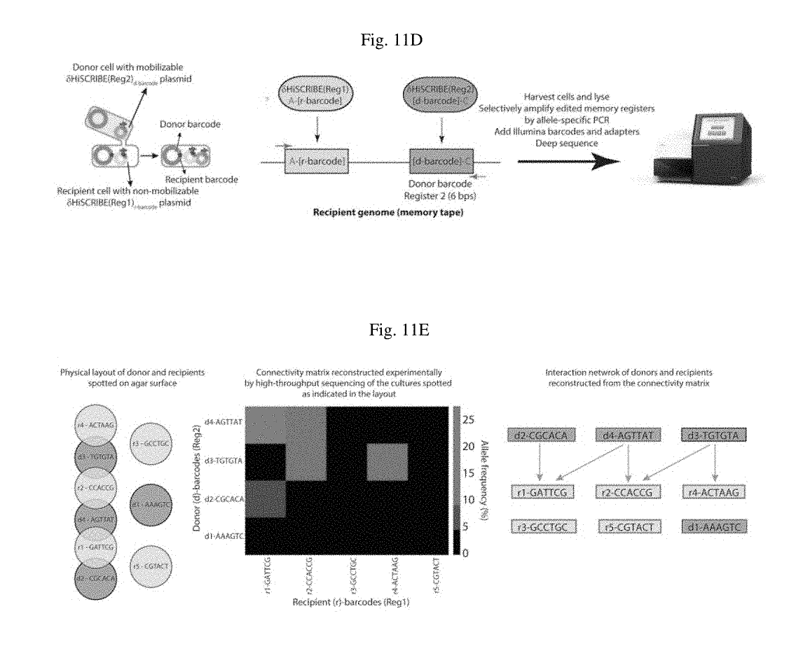

To demonstrate the applicability of the SCRIBE system for DNA writing in non-traditional hosts, this system was used for genome editing in Pseudomonas putida (P. putida). To this end, the SCRIBE(upp)OFF plasmids targeting either the lagging strand or the leading strand of the uracil phosphoribosyltransferase (upp) ORF were designed to introduce two premature stop codons into this ORF, thus making cells insensitive to 5-fluorouracil (5-FU). SCRIBE cassettes were cloned into a broad-host-range plasmid (harboring the pBBR1 origin of replication) and transformed into the P. putida KT2440 strain. Recombinant frequency was assayed by measuring the ratio of cells resistant to 5-FU to viable cells. While targeting the leading strand did not result in a significant increase in the editing efficiency, targeting the lagging strand improved the editing efficiency by about two orders of magnitude, demonstrating that SCRIBE is functional in P. putida (FIG. 8). The editing efficiency may be further improved by using strategies described in this work, including knocking out homologs of recJ and xonA in P. putida (or knocking down these genes using CRISPRi), counterselection by CRISPR-Cas9 nucleases, and using homologs of Beta that are more active in Pseudomonas.

Next, the DNA writing frequency was assessed in the entire population using a screenable plating assay, and observed that more than 99% of transformants (colony forming units (CFUs)) in the population underwent successful DNA editing after receiving the δHiSCRIBE plasmid (FIGS. 2E-2H). Similar to the previous experiment, more than 99% of WT alleles within each CFU were converted into mutated alleles within 2 days (˜60 generations). These results demonstrate that δHiSCRIBE is a highly efficient, broadly applicable, and scarless genome writing platform that can achieve ˜100% editing efficiency at both single-cell and population-level without requiring any cis-encoded sequence on the target, double-strand DNA breaks, or selection.

To systematically assess δHiSCRIBE writing efficiency in an entire population, a screening assay with colorimetric readout was used. Two stop codons were introduced into the galK ORF of the MG1655 ΔrecJ ΔxonA (exo− galKOFF) reporter strain. These reporter cells were transformed with δHiSCRIBE(galK)_{ON} (δHiSCRIBE plasmid encoding ssDNA identical to the WT galK). These cells were recovered for one hour in LB (37 C, 300 RPM) and plated on MacConkey+galactose (gal)+antibiotic plates in order to select for transformants. The conversion of the galK_{OFF} allele to galK_{ON} (i.e., the WT allele) was monitored by scoring the color of transformant colonies. As shown in FIGS. 2E-2H, all the galK_{OFF} (white) cells transformed with the δHiSCRIBE(galK)_{ON} plasmid formed galactose-fermenting galK_{ON} (pink) colonies on the indicator plates. No pink colonies were detected when cells were transformed with a non-specific δHiSCRIBE (δHiSCRIBE(NS)) plasmid. These results demonstrate that in the entire population of cells that received the δHiSCRIBE(galK)_{ON} plasmid, galK_{OFF} alleles were converted to galKON over the course of colony growth, resulting in a phenotypic change in colony color.

Since Beta-mediated recombineering is a replication-dependent process, the conversion of galKOFF to galKON occurs over the course of growth of the colonies, and a single pink colony observed on a transformation plate may contain a heterogeneous population of both edited and non-edited alleles. The frequency of these alleles within single colonies by PCR amplification of the galK locus followed was measured by Sanger sequencing as well as high-throughput sequencing. To avoid any difference in fitness between the two alleles in the presence of galactose, after the δHiSCRIBE(galK)_{ON} plasmid were transformed into exo− galK_{OFF} reporter cells, transformants were selected on LB plates, instead of MacConkey+gal plates. Sanger sequencing of PCR amplicons of the galK locus obtained from these transformants showed a mixture of peaks in the target site, suggesting that each colony on these plates may have contained a mixture of edited and non-edited alleles (FIGS. 2E-2H). To give the replication-dependent δHiSCRIBE writing system additional time to work, the colonies were restreaked on fresh plates. Sanger sequencing of galK locus amplicons obtained from these colonies indicated the full conversion of galK_{OFF} allele to galK_{ON}, to the extent that the galK_{OFF} allele was below the limit of detection. These results were further quantified and validated by high-throughput sequencing of galK amplicons. These results indicate that δHiSCRIBE system can be used to edit a desired genomic locus up to homogeneity (˜100% efficiency) in an entire population, and without the requirement for any double-strand DNA breaks and cis-encoded elements on the target.

### Example 5: Continuous Evolution of Genomic Loci

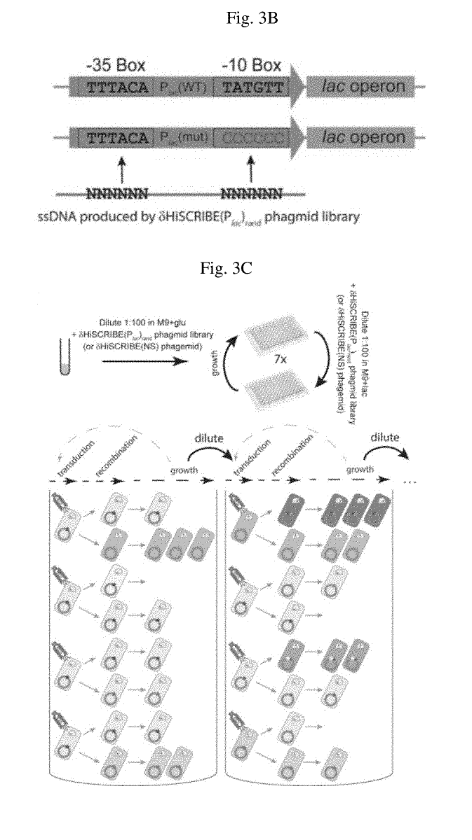

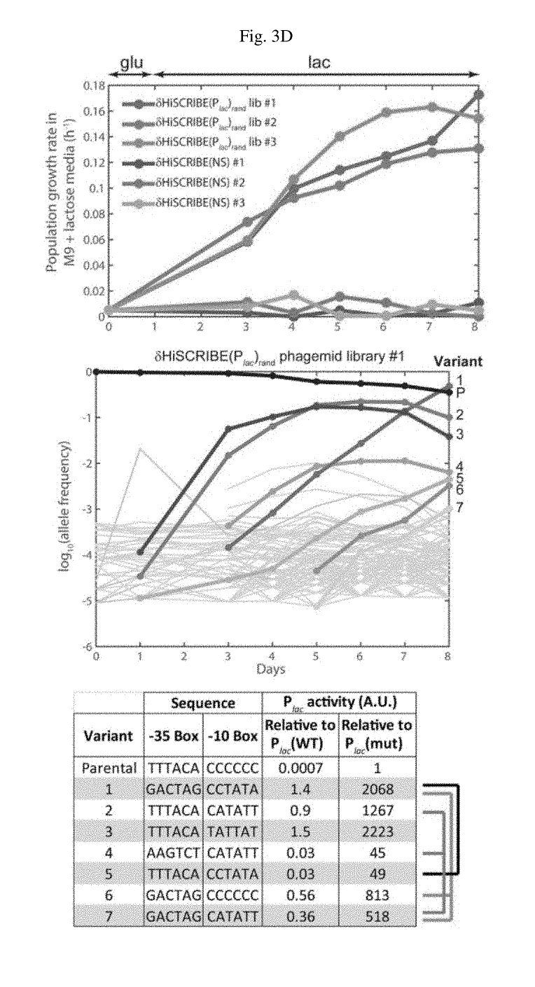

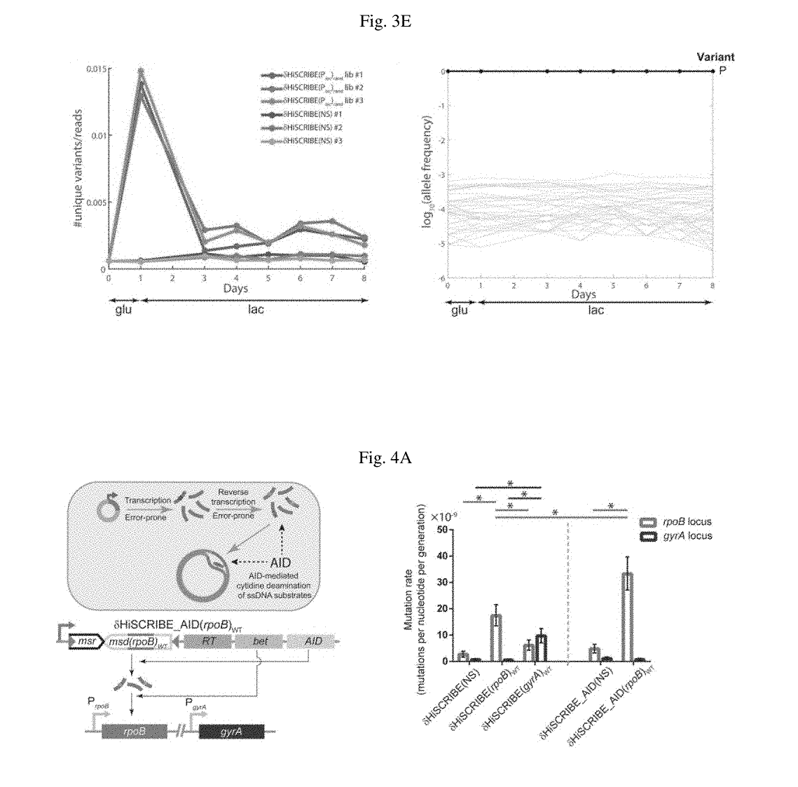

Evolution is a continuous process of genetic diversification and phenotypic selection that tunes the genetic makeup of living organisms and maximizes their fitness in a given environment over evolutionary timescales. Evolutionary design is a powerful approach for engineering living systems. Acting as analog sensors, living cells continuously sense and respond to environmental cues to optimize their fitness in a given environment. Depending on the time-scale of these cues, cells response could vary. While short-term cues are often responded by regulation of transcriptional and translational programs, the response to cues that last within evolutionary time-scales are often in the form of permanent genetic changes. Accumulation of these genetic changes over evolutionary time-scales would lead to adaptive genetic changes that result in increase of fitness of the organism in a given environment. Increased in fitness in turn results in faster replication and amplification of the associated genotype. The power of evolutionary process can be harnessed in the lab in the form of iterative cycles of diversity generation and screening. Nonetheless, due to practical limitations, with the in vitro diversity generation techniques, often very few cycles of directed evolution are feasible. Techniques that enable parallel and continuous cycles of evolution are key enablers towards harnessing the power of evolution in practical timescales in a lab. Continuous evolution could be achieved by the in vivo production of variants of a desired network and coupling it to a continuous selection setup. The ability to conditionally change information stored on a genome is a powerful strategy to dynamically control and engineer cellular phenotypes. Using evolutionary strategy for tuning cellular traits and driving cells towards certain evolutionary trajectories is only viable in evolutionary time-scales and not that practical in laboratory settings. The engineered constructs of the present disclosure provide a tractable tool for linking cellular and environmental cues to high-efficiency genomic editing and cellular fitness. Efficient DNA writers can enable the continuous and targeted diversification of desired loci in vivo in a temporally- and spatially-programmable manner. Targeted diversity generation can be coupled with a continuous selection or screening setup to achieve adaptive writing and tune cellular fitness continuously and autonomously with minimal human intervention (FIG. 3A).

Thus, further described herein is the tuning of cellular fitness and acceleration the rate of evolution of a desired target site by linking the high-efficiency genomic editing constructs to a continuous selection/screening setup. To demonstrate this with HiSCRIBE DNA writers, cellular fitness (i.e., growth rate) was linked to a cell's ability to consume lactose (lac) as the sole carbon source. To enable a wide dynamic range in fitness to be explored, the activity of the native lac operon promoter (P_{lac}) was first weakened by introducing mutations into its −10 box (P_{lac}(mut), FIG. 3B) in the MG1655 exo⁻ strain. Cells with the P_{lac}(mut) promoter (hereafter referred to as the parental strain) grew poorly in minimal media (M9) when lactose was present as the sole carbon source. Then, a randomized δHiSCRIBE phagemid library (δHiSCRIBE(P_{lac})_{rand}) was used to continuously introduce diversity into the −10 and −35 sequences of this promoter (FIG. 3B). Starting from an overnight culture, parental cells were diluted into M9+glucose media and divided into two groups, which were then treated with phagemid particles from either a δHiSCRIBE(P_{lac})_{rand} library or δHiSCRIBE(NS). After this initial growth in glucose, cells were diluted and regrown in M9+lactose in the presence of phagemid particles for six additional rounds to allow for concomitant diversification, selection, and propagation of beneficial mutations (FIG. 3C). As shown in FIG. 3D (top panel), the overall growth rates of cell populations in lactose increased when they were transduced with the δHiSCRIBE(P_{lac})_{rand} phagemid library. In contrast, the growth rates of cell populations exposed to the control δHiSCRIBE(NS) phagemid particles did not change over time. These results demonstrate that the δHiSCRIBE library can introduce targeted diversity into desired loci (−10 and −35 boxes of the P_{lac} promoter) that result in fitness increases of the population under selection over relatively short timescales, and much faster that what can be achieved by natural Darwinian evolution (i.e., in cells transformed with non-targeting δHiSCRIBE(NS)).

To monitor the dynamics of mutants in these cultures, the P_{lac} region was amplified by PCR and deep sequencing was performed at different time points over the course of the experiment. The diversity and frequency of P_{lac} alleles in samples that had been exposed to the δHiSCRIBE(NS) phagemid did not change significantly over time and the parental allele comprised ˜100% of the population at all analyzed time points (FIG. 3E). Further inspection of the rare variants observed in these samples revealed mostly single nucleotide changes compared to the parental allele, suggesting that these arose from sequencing errors. On the other hand, the diversity of P_{lac} alleles greatly increased in cultures that were exposed to the δHiSCRIBE(P_{lac})_{rand} phagemid library when they were initially grown in the M9+glucose condition (FIG. 3E). This initial increase in allele diversity was followed by a significant drop upon dilution of cells in lactose media, likely due to sampling drift and strong selection for alleles that allow for lactose metabolism. Throughout the experiment, however, the number of unique variants remained significantly higher in the δHiSCRIBE(P_{lac})_{rand} cultures than in the negative controls. Moreover, the frequency of P_{lac} alleles from samples that had been exposed to δHiSCRIBE(P_{lac})_{rand} changed dynamically over time (FIG. 3D, middle panel). Notably, by the end of the experiment, the frequency of the parental allele dropped to less than 50% and one variant (variant #1) became the dominant allele in the population. Further analysis of frequent variants within the diversified population indicated that multiple mutations occurred in the −10 and −35 boxes in discrete steps, in which secondary mutations arising on top of primary mutations led to an increase in fitness (FIG. 3D, bottom panel). For example, based on allele enrichment and P_{lac} activity data (see below), the dominant allele (variant #1) was likely produced from an initial, less active mutant (variant #5) and subsequently took over the population based on increased fitness (i.e., P_{lac} activity). The sequences of successful variants that evolved in our experiments were especially AT-rich (FIG. 3D, bottom panel, and FIGS. 9A and 9B), as is expected from the canonical sequences of these regulatory elements in E. coli.

To validate that the identified variants were indeed responsible for increases in fitness, these variants were reconstructed in the parental strain background and assessed their activity by measuring β-galactosidase activity. As shown in FIG. 3D (bottom panel), all the evolved variants showed a significant increase in β-galactosidase activity over the parental variant, indicating successful tuning of the activity of the P_{lac} promoter. For example, the dominant variant at the end of the experiment (variant #1) exhibited a >2000-fold increase in β-galactosidase activity relative to the parental strain, corresponding to a 1.4-fold increase over the wild-type P_{lac} promoter.

These results demonstrate that, once coupled to a continuous selection or screen, HiSCRIBE can be used for adaptive writing and continuous and autonomous diversity generation in desired target loci, enabling easy and flexible continuous evolution experiments requiring minimal human intervention. In the current setup, the continuous diversity generation system relies on the continuous and multiplexed (FIG. 7) delivery of phagemid-encoded HiSCRIBE variants that compete for writing on the target locus once inside the cells. Further incorporating a conditional origin of replication into phagemids or conjugative plasmids may help to increase the rate of evolution by enforcing writing and curing steps in a more controlled fashion.

### Example 6: In Vivo Targeted Mutagenesis

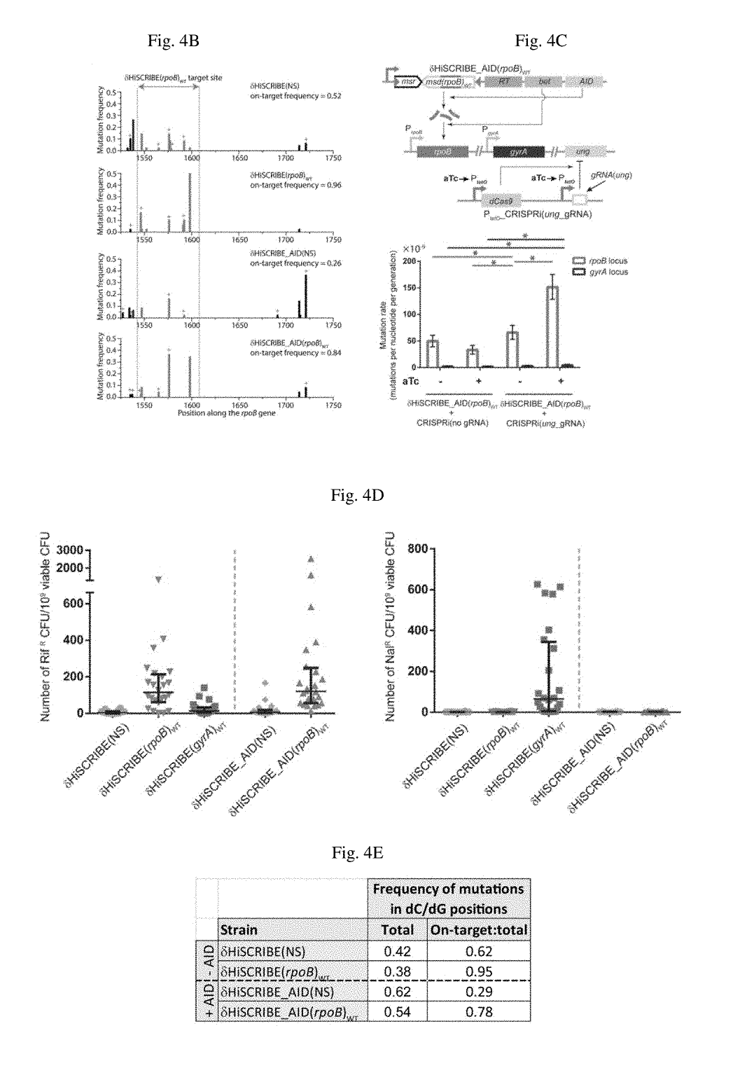

Evolutionary design is a powerful approach for engineering living systems, however, in many cases, the natural rate of mutagenesis is not high enough to allow making necessary genetic changes accessible on practical timescales in a lab. Platforms that enable to selectively increase the mutation rate in a desired genomic locus without increasing the global mutation rate, could enable engineering cellular evolvability and facilitate harnessing power of evolution for engineering living cells. Since transcription and reverse-transcription processes have a lower fidelity than DNA replication, it was investigated if this lower fidelity could be leveraged to increase the mutation rate of a target site without affecting mutation rate of the rest of a genome, by producing a library ssDNA variants in vivo followed by recombination of these variants into the target genomic site (FIG. 4A).

A well-established plating assay and fluctuation analysis was used to measure locus-specific de novo mutation rates induced by HiSCRIBE at targeted and non-targeted loci. Using this assay, mutation rates at two different loci, rpoB and gyrA, were estimated based on the frequency of rifampicin-resistant (RifR) and nalidixic acid-resistant (NalR) cells in the population, respectively. Specifically, locus-specific mutation rates were measured in MG1655 exo− cells harboring δHiSCRIBE(rpoB)WT (which encodes a 72-bp ssDNA with the same sequence as WT rpoB), δHiSCRIBE(gyrA)WT (which encodes a 72-bp ssDNA with the same sequence as WT gyrA), or δHiSCRIBE(NS). Targeting δHiSCRIBE to rpoB increased the mutation rate at this locus (measured by the frequency of RifR mutants) while having a minimal effect on the mutation rate at the gyrA locus (measured by the frequency of NalR mutants) (FIGS. 4A and 4D). Similarly, expressing δHiSCRIBE(gyrA)WT resulted in a significant increase in the mutation rate at the gyrA locus while having a minimal effect on the mutation rate at the rpoB locus. These results suggest that HiSCRIBE can selectively increase the mutation rate of a desired target site without increasing the background mutation rate.

Next, whether the rate or spectrum of targeted mutations could be modulated by overexpressing an ssDNA-specific modifying enzyme such as human activation-induced cytidine deaminase (AID) was investigated. AID is an ssDNA-specific cytidine deaminase that is involved in the diversification of the immunoglobulin locus in vertebrates and was previously shown to retain its functionality to deaminate cytidine in E. coli. AID could act on ssDNA substrates produced by HiSCRIBE and/or on unwound ssDNA segments generated during passage of the replication fork and are likely to be more accessible due to the presence of recombineering factors. As shown in FIG. 4A, overexpression of AID alongside δHiSCRIBE(rpoB)WT from a synthetic operon (hereafter referred to as δHiSCRIBE_AID(rpoB)_{WT}) increased the targeted mutation rate of the rpoB locus even further. However, it also slightly increased the background mutation rate as measured by the NalR phenotype at the gyrA locus, likely due to non-specific action of AID on genomic DNA

To identify the nature of the identified mutants, the rpoB locus of fifty RifR colonies from each strain was Sanger-sequenced and the observed frequency of each mutation versus its position along the rpoB gene was plotted (FIG. 4B). In cells expressing δHiSCRIBE(rpoB)_{WT}, RifR mutations were almost exclusively observed in the 72 bp target region. However, in cells expressing δHiSCRIBE(NS), RifR mutations occurred both inside and outside of this region. This suggests that δHiSCRIBE(rpoB)_{WT} not only increased the mutation rate of the rpoB locus, but more specifically did so by elevating the mutation rate within the target region defined by the δHiSCRIBE template. Consistent with the previous reports, overexpression of AID increased frequency of mutations at dC/dG positions (FIG. 4B and FIG. 4E). In cells expressing δHiSCRIBE_AID(rpoB)_{WT}, most mutations in dC/dG positions were observed within the 72 bp target window. This observation was in contrast to cells expressing δHiSCRIBE_AID(NS), where such mutations were observed mostly outside of the targeted region. These results demonstrate that HiSCRIBE can selectively increase the mutation rate at a desired target locus, and that the spectrum of mutations can be tuned by using ssDNA-modifying enzymes.

In order to increase the targeted mutation rate even further, the uracil DNA glycosylase gene (ung) of E. coli, which is responsible for the repair of deaminated cytidines, was conditionally knocked down with an aTc-inducible CRISPRi system. As shown in FIG. 4C, a significant increase in the mutation rate of the targeted locus (rpoB) was observed in cells expressing both δHiSCRIBE_AID (rpoB)WT and CRISPRi (ung_gRNA) upon induction of the CRISPRi system. The background mutation rate in the non-targeted locus (gyrA), measured by the NalR phenotype, was not significantly affected. These results suggest that by conditionally knocking down systems that repair introduced lesions, one can increase the rate of targeted mutations without affecting the global mutation rate. Targeted diversity generation could be further augmented by additional strategies, including using error-prone RNA polymerases and reverse-transcriptases, RNA and ssDNA modifying enzymes, and/or conditionally suppressing machinery involved in the repair of corresponding lesions (e.g., MMR) using CRISPRi. These targeted de novo mutagenesis strategies, which as opposed to using a generalized hypermutator genetic background or mutagen chemicals could elevate mutation rate of desired loci without increasing global mutation rate, could have broad utility in evolutionary engineering applications.

### Example 7. Recording Spatial Information into DNA Memory

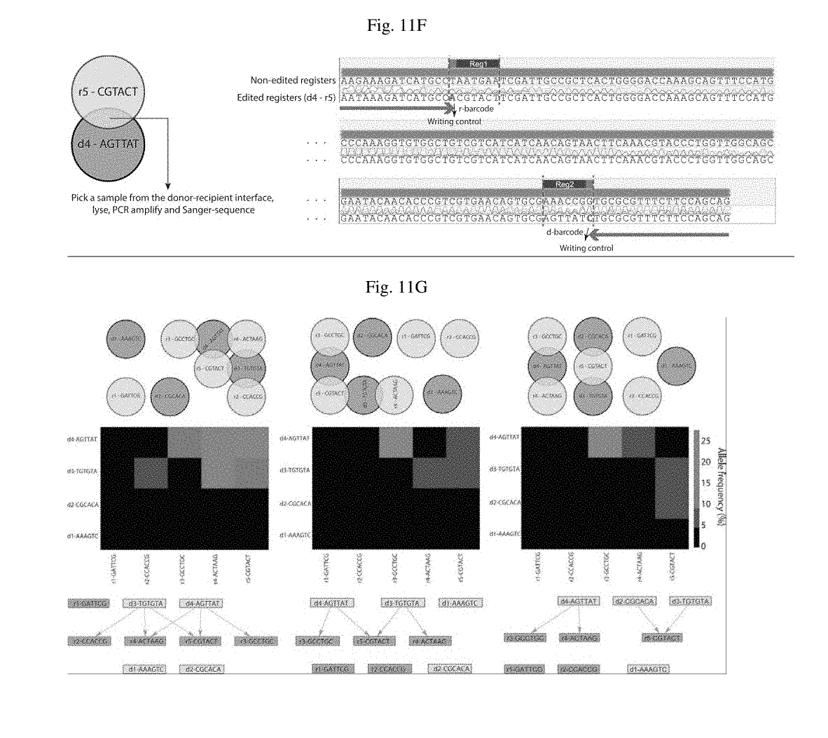

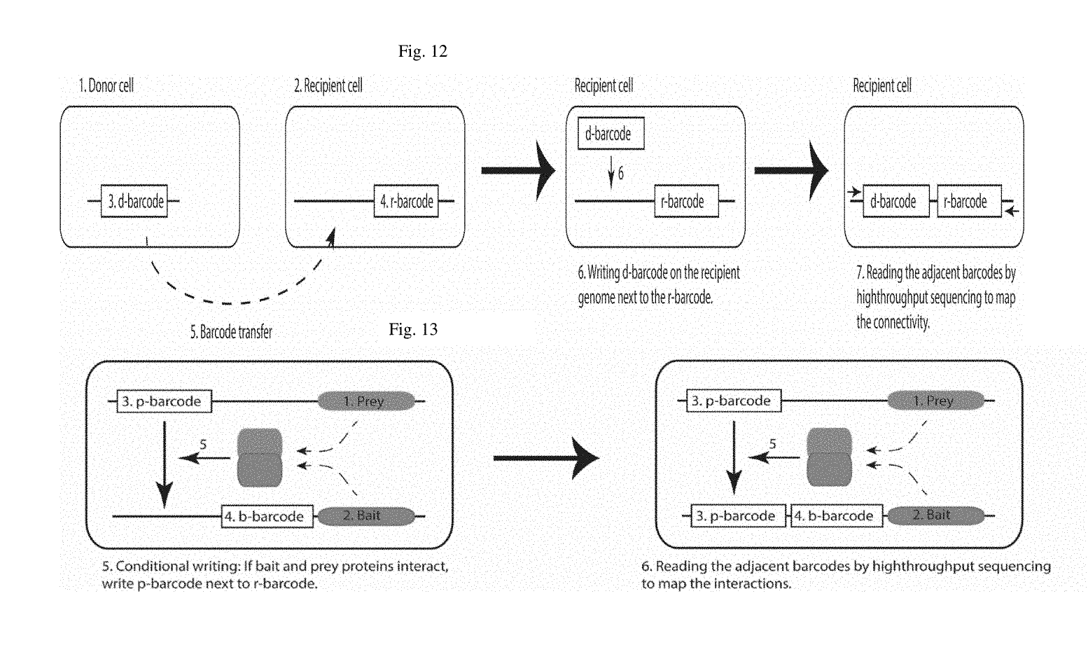

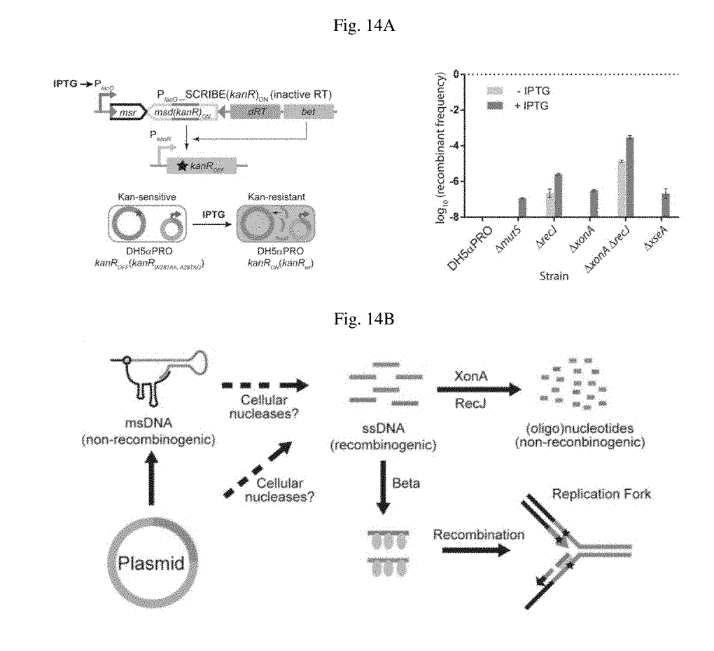

Many events and interactions that occur in biological systems, such as cell-cell interactions, are transient and thus hard to study in high throughput or with high resolution. If transient interactions are permanently recorded in DNA, they could be mapped by high-throughput sequencing even after samples are disrupted. Conjugation events within a bacterial population were mapped as an example of a “cellular connectome”. MG1655 exo⁻ galK_{OFF} cells were first transformed with a SCRIBE(Reg1) library, which encoded an ssDNA library with 6 randomized nucleotides targeting a 6 bp region (Register 1) within the galK locus. SCRIBE(Reg1) was used to write unique barcodes into the genome of these cells to make a barcoded recipient population (FIG. 11A). A conjugative SCRIBE library (SCRIBE(Reg2)), which targets a 6 bp sequence (Register 2) neighboring Register 1, was transformed into MFDpirPRO cells to make a conjugation donor library. Upon successful conjugation, Register 2 in recipient cells was expected to be written with a unique barcode and thus, sequencing the consecutive Register 1 and Register 2 from the recipient genomes should yield a record of this interaction. To test this method of recording mating interactions, donor and recipient populations were mixed and spotted on nitrocellulose filters on a solid agar surface to allow for conjugation of the SCRIBE(Reg2) library from donors to recipients (FIG. 11A). Samples were then disrupted and grown in liquid cultures to allow for propagation of the conjugated alleles and finalized writing in the memory registers. The two neighboring DNA memory registers were then amplified as a single amplicon by PCR, depleted from non-edited registers by enzymatic digestion of DNA still containing parental restriction sites, and deep sequenced (see Methods). Connectivity matrices between members of donor and recipient populations were then deduced based on the DNA barcodes obtained in the two specified memory registers (FIG. 11B). In order to estimate the rate of false positives due to sequencing errors or spontaneous mutations, connectivity matrices were calculated for two other 6-bp regions within the galK locus that were not targeted by SCRIBE. Only a limited number of connections were detected, and further inspection of the barcodes revealed mostly single-bp differences with the non-edited registers, suggesting that these arose from sequencing errors, which are reportedly ˜10⁻³-10⁻² mutations/nucleotide. False positives could be reduced by using error-reducing library preparations, computational correction methods, and/or more accurate sequencing platforms.

These results demonstrate that transient information, such as cell-cell mating events between bacterial strains, can be memorized in DNA for later retrieval by sequencing. For example, using two 6-bp barcodes, up to 412≈1.67×10⁷ bits of spatial information can be recorded in DNA for later retrieval by sequencing. The system's storage capacity can be scaled up by using longer barcodes (e.g., a Zettabyte of information can be recorded in a 36-bp piece of DNA), thus enabling unprecedented dynamic recording of biologically relevant information in living cells.

SCRIBE may be encoded in phages, conjugative plasmids or other mobile genetic elements and designed to write similar barcodes near identifiable genomic signatures (e.g., 16S rRNA gene) to assess the in situ host range of these mobile elements. While only pairwise interactions were recorded in this experiment, in principle, multiple interactions can be recorded into adjacent DNA registers to facilitate the mapping of multidimensional interactomes with high-throughput sequencing, particularly as sequencing fidelity and read length continue to improve. This is useful, for example, when mapping interaction networks with more than two counterparts, e.g., protein-protein interactions in a protein complex or neural connectome mapping. Furthermore, extending this approach to mammalian cells using analogous high-efficiency genome editing technologies, such as CRISPR-Cas9, will enable use of this genome editing system to record spatiotemporal interactions, such as neural connectomes, or transient events, such as protein-protein interactions, in a high-throughput fashion.

The concept of recording Spatial Information into DNA Memory was demonstrated by mapping conjugation events between bacterial populations. To this end, two neighboring 6 bp sequences on the galK locus were first designated as memory registers. Then, a series of δHiSCRIBE(Reg1)r-barcode and δHiSCRIBE(Reg2)d-barcode plasmids were constructed, each encoding a different barcoded ssDNA template. These plasmids each write a unique 7 bp DNA sequence (1 bp writing control+6 bp barcode) on the first and the second registers, respectively (FIG. 11D). The writing control nucleotide was designed as a mismatch to the unedited register and used to selectively amplify edited registers (see Methods). The δHiSCRIBE(Reg1)r-barcode plasmids was introduced into the MG1655 exo− strain to make a set of conjugation recipient cells. Upon transformation, these plasmids write a unique barcode in the first genomic register in these cells (Register 1), and uniquely mark these recipient populations. δHiSCRIBE(Reg2)d-barcode plasmids, harboring a RP4 origin of transfer, were transformed into MFDpirPRO cells to make a set of conjugation donor populations. Upon successful conjugation and transfer from donor to recipient, these plasmids write a unique barcode in the Register 2 in recipient cells. Thus, sequencing the consecutive Register 1 and Register 2 in recipient genomes yield a record of this interaction (FIG. 11D). Using this barcode joining strategy, it was demonstrated that the interaction between a barcoded donor population and a barcoded recipient population could be successfully recorded and faithfully retrieved by allele-specific PCR of conjugation mixtures followed by Sanger sequencing (FIGS. 11F and 11G). To this end, a donor population was spotted with a single donor barcode on filter paper, overlapped it with another filter paper with a recipient population containing a single recipient barcode, and then confirmed that our retrieval process was correct (FIG. 11F). More complex spatial layouts were then constructed by overlapping multiple different barcoded donor populations and barcoded recipient populations. It was demonstrated that allele-specific PCR combined with high-throughput sequencing could faithfully retrieve conjugative interactions between the distinct barcoded donor and recipient populations laid down in different patterns (FIGS. 11E and 11G).

### Materials and Methods

### Strains and Plasmids

Conventional cloning methods were used to construct the plasmids. Lists of strains and plasmids used in this study are provided in Tables 1 and 2, respectively. The sequences for the synthetic parts are provided in Tables 3.

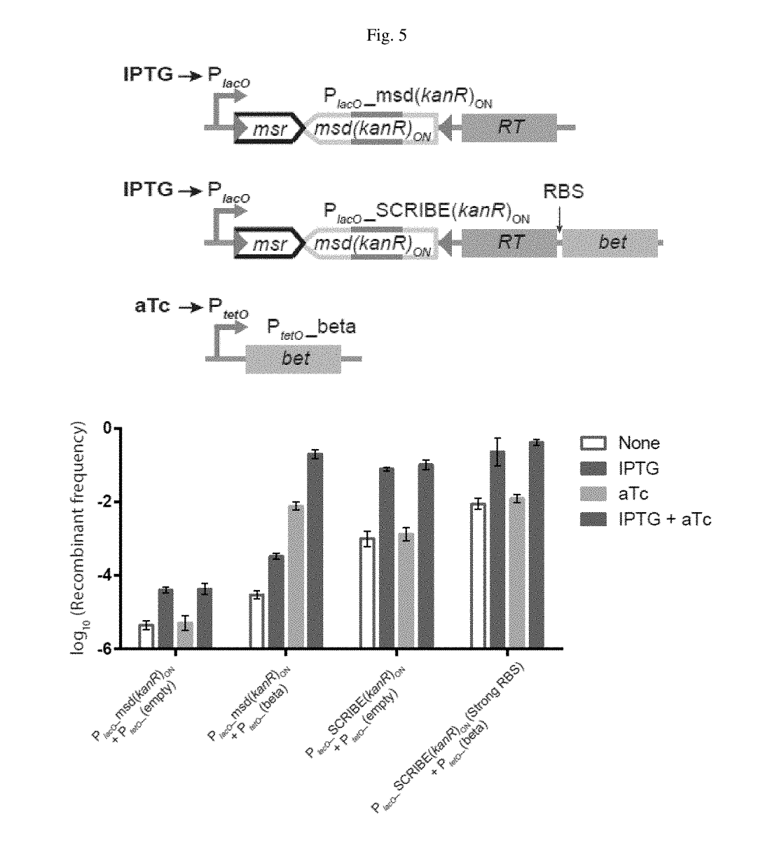

TABLE 2
List of the reporter strains used in this study

| | Strain | | |
|---|---|---|---|
| Name | Code | Genotype | Used in |
| kanR_{OFF} reporter | FFF144 | DH5αPRO galK::kanR_{W28TAA, A29TAG} | FIG. 1A |
| strain | | | FIG. 1C |
| kanR_{OFF} ΔmutS | FFF524 | DH5αPRO ΔmutS galK::kanR_{W28TAA, A29TAG} | FIG. 1A |
| kanR_{OFF} ΔrecJ | FFF525 | DH5αPRO ΔrecJ galK::kanR_{W28TAA, A29TAG} | FIG. 1A |
| kanR_{OFF} ΔxonA | FFF527 | DH5αPRO ΔxonA galK::kanR_{W28TAA, A29TAG} | FIG. 1A |
| kanR_{OFF} ΔxseA | FFF590 | DH5αPRO ΔxseA galK::kanR_{W28TAA, A29TAG} | FIG. 1A |
| kanR_{OFF} ΔrecJ | FFF589 | DH5αPRO ΔrecJ ΔxonA galK::kanR_{W28TAA, A29TAG} | FIG. 1A FIG. 5 |
| ΔxonA | | | FIG. 6 |
| MG1655 exo reporter | FFF964 | MG1655 ΔrecJ ΔxonA | FIGS. 4A-4E |
| strain | | | |
| galK_{OFF} reporter | FFF1087 | MG1655 ΔrecJ ΔxonA galK_{L187TAA, L188TGA} | FIG. 1D FIG. |
| strain | | | 1E FIG. 4 |
| galK_{OFF} reporter | FFF1086 | MG1655 galK_{L187TAA, L188TGA} | FIG. 2A FIG. |
| strain | | (For transduction experiment, F plasmid (from DH5α F⁺) | 2B |
| | | was introduced to this strain via conjugation) | |
| galK_{OFF} St^{R} reporter | FFF1296 | MG1655 St^{R} galK_{L187TAA, L188TGA} | FIG. 2A FIG. |
| strain | | (For transduction experiment, F plasmid (from DH5α F⁺) | 2C FIG. 2D |
| | | was introduced to this strain via conjugation) | |
| galK_{OFF} lacZ_{OFF} | FFF1265 | MG1655 ΔrecJ ΔxonA galK_{L187TAA, L188TGA} lacZ_{A35TAA, S36TAG} | FIG. 7 |
| reporter strain | | (For transduction experiment, F plasmid (from DH5α F⁺) | |
| | | was introduced to this strain via conjugation) | |
| MG1655 P_{lac}(mut) | FFF1032 | MG1655 P_{lac(mut)} where −10 Box of P_{lac} promoter is | FIG. 3 |
| | | mutated from TATGTT to CCCCC | FIG. 9 |
| | | (For transduction experiment, F plasmid (from CJ236 | |
| | | (NEB)) was introduced to this strain via conjugation) | |
| MFDₚᵢᵣ | FFF1040 | MG1655 | (39) |
| | | RP4-2-Tc::[Mu1::aac(3)IV-ΔaphA-Δnic35-ΔMu2::zeo] | |
| | | ΔdapA::(erm-pir) ΔrecA | |
| | | PRO plasmid (pZS4Int-lacI/tetR, Expressys) was | |
| | | transformed to this strain to make a PRO version | |
| Pseudomonas putida | FFF480 | | FIG. 8 |
| KT2440 | | | |

TABLE 3
List of the plasmids used in this study

| Name | Plasmid Code | Maker | Used in | Described in |
|---|---|---|---|---|
| PRO Plasmid (pZS4Int-lacI/tetR) | pFF187 | Spe/Str | FIGS. 11A-11G | Expressys |
| | | | | (44) |
| pKD46 | pFF59 | Carb | FIG. 2A | (2) |
| P_{lacO}_msd(kanR)_{ON} | pFF530 | Cm | FIG. 5 | (13) |
| P_{tetO}_bet | pFF145 | Carb | FIG. 5 | (13) |
| P_{lacO}_SCRIBE(kanR)_{ON} | pFF745 | Cm | FIG. 1A | (13) |
| | | | FIG. 5 | |
| P_{lacO}_SCRIBE(kanR)_{ON}_dRT | pFF755 | Cm | FIG. 1A | (13) |
| P_{lacO}_SCRIBE(kanR)_{ON} | pFF804 | Cm | FIG. 5 | This work |
| (Strong RBS) | | | | |
| P_{lacO}_SCRIBE(kanR)_{ON} | pFF944 | Carb | FIG. 1C | This work |
| (Strong RBS) | | | FIG. 6 | |
| P_{tetO}_CRISPRi(no gRNA) | pFF1156 | Cm | FIG. 1C | (22) |
| | | | FIG. 1E | Addgene |
| | | | | #44249 |
| P_{tetO}_CRISPRi(recJ gRNA & xonA gRNA) | pFF1165 | Cm | FIG. 1C | This work |
| Pₜₑₜ₀₋CRISPRi(ung gRNA) | pFF1369 | Cam | FIG. 4C | This work |
| SCRIBE(galK)_{ON} (Strong RBS) | pFF1081 | Carb | FIG. 1D | This work |
| SCRIBE(galK)_{ON}_P_{tetO}_gRNA(galK_{OFF}) | pFF1220 | Carb | FIG. 1E | This work |
| P_{tetO}_Cas9 | pFF1172 | Cm | FIG. 1E | This work |
| SCRIBE(galK)_{ON}_CRISPRi(recJ gRNA & | pFF1298 | Carb | FIG. 2 | This work |
| xonA gRNA) | | | | |
| SCRIBE(rpoB) (Strong RBS) | pFF1328 | Kan | FIG. 4 | This work |
| SCRIBE(gyrA) (Strong RBS) | pFF1336 | Kan | FIG. 4 | This work |
| SCRIBE_AID(rpoB) | pFF1329 | Kan | FIG. 4 | This work |
| (Strong RBS) | | | | |
| δHiSCRIBE(lacZ)_{ON} | pFF1299 | Carb | FIG. 7 | This work |
| (Strong RBS) | | | | |
| SCRIBE(lacZ)_{ON} (Strong RBS) | pFF1084 | Carb | FIG. 7 | This work |
| SCRIBE(Upp)_{OFF(leading)} (Strong RBS) | pFF1113 | Kan | FIG. 8 | This work |
| SCRIBE(Upp)_{OFF(lagging)} (Strong RBS) | pFF1114 | Kan | FIG. 8 | This work |
| SCRIBE(Upp)_{OFF(lagging)}_CRISPRi(recJ gRNA | pFF1145 | Kan | FIG. 8 | This work |
| & xonA gRNA) (Strong RBS) | | | | |

TABLE 4
List of the synthetic parts and their
corresponding sequences used in this study

| | | | SEQ | |
|---|---|---|---|---|
| | | | ID | |
| Part name | Type | Sequence | NO. | Ref |
| P_{lacO} | Promoter | AATTGTGAGCGGATAACAATTGACATTGTGAGCGG | 4 | (44) |
| | | ATAACAAGATACTGAGCACATCAGCAGGACGCAC | | |
| | | TGACC | | |
| P_{tetO} | Promoter | TCCCTATCAGTGATAGAGATTGACATCCCTATCAG | 5 | (44) |
| | | TGATAGAGATACTGAGCACATCAGCAGGACGCAC | | |
| | | TGACC | | |
| msr | Primer for the | ATGCGCACCCTTAGCGAGAGGTTTATCATTAAGGT | 6 | (13) |
| | RT | CAACCTCTGGATGTTGTTTCGGCATCCTGCATTGA | | |
| | | ATCTGAGTTACT | | |
| msd(kanR)_{ON} | Template for | GTCAGAAAAAACGGGTTTCCTGAATTCCAACATGG | 7 | (13) |
| | the RT | ATGCTGATTTATATGGGTATAAATGGGCCCGCGAT | | |
| | | AATGTCGGGCAATCAGGTGCGACAATCTATCGGAA | | |
| | | TTCAGGAAAACAGACAGTAACTCAGA | | |
| msd(galK)_{ON} | Template for | GTCAGAAAAAACGGGTTTCCTGAATTCCAGCTAAT | 8 | (13) |
| | the RT | TTCCGCGCTCGGCAAGAAAGATCATGCCCTCTTGA | | |
| | | TCGATTGCCGCTCACTGGGGACCAAAGCAGTTTCC | | |
| | | GAATTCAGGAAAACAGACAGTAACTCAGA | | |
| msd(lacZ)_{ON} | Template for | GTCAGAAAAAACGGGTTTCCTGAATTCACCCAACT | 9 | (13) |
| | the RT | TAATCGCCTTGCAGCACATCCCCCTTTCGCCAGCT | | |
| | | GGCGTAATAGCGAAGAGGCCCGCACCGATCGCCC | | |
| | | TGAATTCAGGAAAACAGACAGTAACTCAGA | | |
| RT Ec86 | Reverse | As described in (13) | | (13) |
| | Transcriptase | | | |
| Beta | ssDNA-specific | As described in (13) | | (13) |
| | recombinase | | | |
| | protein | | | |
| kanR_{OFF} | Reporter gene | As described in (13) | | (13) |
| galK_{OFF} | Reporter gene | ATGAGTCTGAAAGAAAAAACACAATCTCTGT | 28 | (13) |
| | The two | TTGCCAACGCATTTGGCTACCCTGCCACTCAC | | |
| | premature | ACCATTCAGGCGCCTGGCCGCGTGAATTTGAT | | |
| | stop codons | TGGTGAACACACCGACTACAACGACGGTTTC | | |
| | in this ORF | GTTCTGCCCTGCGCGATTGATTATCAAACCGT | | |
| | are underlined. | GATCAGTTGTGCACCACGCGATGACCGTAAA | | |
| | The location | GTTCGCGTGATGGCAGCCGATTATGAAAATCA | | |
| | of Reg1 and | GCTCGACGAGTTTTCCCTCGATGCGCCCATTG | | |
| | Reg2 in this | TCGCACATGAAAACTATCAATGGGCTAACTAC | | |
| | ORF are | GTTCGTGGCGTGGTGAAACATCTGCAACTGCG | | |
| | italicized. | TAACAACAGCTTCGGCGGCGTGGACATGGTG | | |
| | ClaI and | ATCAGCGGCAATGTGCCGCAGGGTGCCGGGT | | |
| | AgeI sites are | TAAGTTCTTCCGCTTCACTGGAAGTCGCGGTC | | |
| | shown in bold. | GGAACCGTATTGCAGCAGCTTTATCATCTGCC | | |
| | | GCTGGACGGCGCACAAATCGCGCTTAACGGT | | |
| | | CAGGAAGCAGAAAACCAGTTTGTAGGCTGTA | | |
| | | ACTGCGGGATCATGGATCAGCTAATTTCCGCG | | |
| | | CTCGGCAAGAAAGATCATGCCTAATGA [symbol] TCGA | | |
| | | TTGCCGCTCACTGGGGACCAAAGCAGTTTCCA | | |
| | | TGCCCAAAGGTGTGGCTGTCGTCATCATCAAC | | |
| | | AGTAACTTCAAACGTACCCTGGTTGGCAGCGA | | |
| | | ATACAACACCCGTCGTGAACAGTGCGAAACCG | | |
| | | GTGCGCGTTTCTTCCAGCAGCCAGCCCTGCGT | | |
| | | GATGTCACCATTGAAGAGTTCAACGCTGTTGC | | |
| | | GCATGAACTGGACCCGATCGTGGCAA [symbol] | | |
| | | GTGCGTCATATACTGACTGAAAACGCCCGCAC | | |
| | | CGTTGAAGCTGCCAGCGCGCTGGAGCAAGGC | | |
| | | GACCTGAAACGTATGGGCGAGTTGATGGCGG | | |
| | | AGTCTCATGCCTCTATGCGCGATGATTTCGAA | | |
| | | ATCACCGTGCCGCAAATTGACACTCTGGTAGA | | |
| | | AATCGTCAAAGCTGTGATTGGCGACAAAGGT | | |
| | | GGCGTACGCATGACCGGCGGCGGATTTGGCG | | |
| | | GCTGTATCGTCGCGCTGATCCCGGAAGAGCTG | | |
| | | GTGCCTGCCGTACAGCAAGCTGTCGCTGAACA | | |
| | | ATATGAAGCAAAAACAGGTATTAAAGAGACT | | |
| | | TTTTACGTTTGTAAACCATCACAAGGAGCAGG | | |
| | | ACAGTGCTGA | | |
| lacZ_{OFF} | Reporter gene | As described in (13) | | (13) |
| beta_RBS | Natural beta | GGTTGATATTGATTCAGAGGTATAAAACGA | 10 | (13) |
| | RBS | | | |
| RBS_A | Strong RBS | AGGAGGTTTGGA | 11 | (45) |
| msd(kanR)_{ON} | Template for | GTCAGAAAAAACGGGTTTCCTGAATTCGGGTATAA | 12 | This |
| (10 bp | the RT | ATGGGCCCGCGATAATGGAATTCAGGAAAACAGA | | work |
| homology arm) | | CAGTAACTCAGA | | |
| msd(kanR)_{ON} | Template for | GTCAGAAAAAACGGGTTTCCTGAATTCTGATTTAT | 13 | This |
| (20 bp | the RT | ATGGGTATAAATGGGCCCGCGATAATGTCGGGCA | | work |
| homology arm) | | ATCGAATTCAGGAAAACAGACAGTAACTCAGA | | |
| msd(kanR)_{ON} | Template for | GTCAGAAAAAACGGGTTTCCTGAATTCACATGGAT | 14 | This |
| (30 bp | the RT | GCTGATTTATATGGGTATAAATGGGCCCGCGATAA | | work |
| homology arm) | | TGTCGGGCAATCAGGTGCGACAGAATTCAGGAAA | | |
| | | ACAGACAGTAACTCAGA | | |
| msd(kanR)_{ON} | Template for | GTCAGAAAAAACGGGTTTCCTGAATTCCAACATGG | 15 | (13) |
| (35 bp | the RT | ATGCTGATTTATATGGGTATAAATGGGCCCGCGAT | | |
| homology arm) | | AATGTCGGGCAATCAGGTGCGACAATCTATCGGAA | | |
| | | TTCAGGAAAACAGACAGTAACTCAGA | | |
| msd(kanR)_{ON} | Template for | GTCAGAAAAAACGGGTTTCCTGAATTCGAGCCATA | 16 | This |
| (80 bp | the RT | TTCAACGGGAAACGTCTTGCTCGAGGCCGCGATTA | | work |
| homology arm) | | AATTCCAACATGGATGCTGATTTATATGGGTATAA | | |
| | | ATGGGCCCGCGATAATGTCGGGCAATCAGGTGCG | | |
| | | ACAATCTATCGATTGTATGGGAAGCCCGATGCGCC | | |
| | | AGAGTTGTTTCTGAAACAGAATTCAGGAAAACAG | | |
| | | ACAGTAACTCAGA | | |
| msd(Upp)_{OFF(leading)} | Template for | GTCAGAAAAAACGGGTTTCCTGAATTCGGTGATCT | 17 | This |
| | the RT | TCTTGCCGGCGATTTTTTCAACCGAGACTCACTAA | | work |
| | | CACCAGCCGTCGATCTCGTAGGTTTCGAGGGGCAG | | |
| | | GAATTCAGGAAAACAGACAGTAACTCAGA | | |
| msd(Upp)_{OFF(lagging)} | Template for | GTCAGAAAAAACGGGTTTCCTGAATTCCTGCCCCT | 18 | This |
| | the RT | CGAAACCTACGAGATCGACGGCTGGTGTTAGTGAG | | work |
| | | TCTCGGTTGAAAAAATCGCCGGCAAGAAGATCACC | | |
| | | GAATTCAGGAAAACAGACAGTAACTCAGA | | |
| msd(rpoB) | Template for | GTCAGAAAAAACGGGTTTCCTGAATTCACCGCCTG | 19 | This |
| | the RT | GGCCGAGTGCGGAGATACGACGTTTGTGCGTAATC | | work |
| | | TCAGACAGCGGGTTGTTCTGGTCCATAAAGAATTC | | |
| | | AGGAAAACAGACAGTAACTCAGA | | |
| msd(gyrA) | Template for | GTCAGAAAAAACGGGTTTCCTGAATTCGAATGGCT | 20 | This |
| | the RT | GCGCCATGCGGACGATCGTGTCATAGACCGCCGAG | | work |
| | | TCACCATGGGGATGGTATTTACCGATTACGAATTC | | |
| | | AGGAAAACAGACAGTAACTCAGA | | |
| msd(P_{lac}) | Template for | GTCAGAAAAAACGGGTTTCCTGAATTCAATGTGAG | 21 | This |
| (highlighted | the RT | TTAGCTCACTCATTAGGCACCCCAGGCNNNNNNCT | | work |
| regions | | TTATGCTTCCGGCTCGNNNNNNGTGTGGAATTGTG | | |
| indicated | | AGCGGATAACAATTTCACACAGGAATTCAGGAAA | | |
| positions in | | ACAGACAGTAACTCAGA | | |
| the msd | | | | |
| corresponding | | | | |
| to the | | | | |
| randomized | | | | |
| -10 and -35 | | | | |
| boxes of P_{lac} | | | | |
| msd(Reg1) | Template for | GTCAGAAAAAACGGGTTTCCTGAATTCGCTAA | 29 | This |
| (underlined | the RT | TTTCCGCGCTCGGCAAGAAAGATCATGCCTNN | | work |
| region | | NNNBTCGATTGCCGCTCACTGGGGACCAAAG | | |
| indicates | | CAGTTTCCATGCGAATTCAGGAAAACAGACA | | |
| positions in | | GTAACTCAGA | | |
| the msd | | | | |
| corresponding | | | | |
| to the | | | | |
| randomized | | | | |
| Register 1 | | | | |
| msd(Reg2) | Template for | GTCAGAAAAAACGGGTTTCCTGAATTCGTTGG | 30 | This |
| (underlined | the RT | CAGCGAATACAACACCCGTCGTGAACAGTGC | | work |
| region | | GNNNNNHGTGCGCGTTTCTTCCAGCAGCCAG | | |
| indicates | | CCCTGCGTGATGTGAATTCAGGAAAACAGAC | | |
| positions in | | AGTAACTCAGA | | |
| the msd | | | | |
| corresponding | | | | |
| to the | | | | |
| randomized | | | | |
| Register 2 | | | | |
| AID | Activated- | ATGGACAGCCTCTTGATGAACCGGAGGAAGTTTCT | 22 | This |
| | induced | TTACCAATTCAAAAATGTCCGCTGGGCTAAGGGTC | | work |
| | Cytidine | GGCGTGAGACCTACCTGTGCTACGTAGTGAAGAGG | | |
| | Deaminase | CGTGACAGTGCTACATCCTTTTCACTGGACTTTGGT | | |
| | | TATCTTCGCAATAAGAACGGCTGCCACGTGGAATT | | |
| | | GCTCTTCCTCCGCTACATCTCGGACTGGGACCTAG | | |
| | | ACCCTGGCCGCTGCTACCGCGTCACCTGGTTCACC | | |
| | | TCCTGGAGCCCCTGCTACGACTGTGCCCGACATGT | | |
| | | GGCCGACTTTCTGCGAGGGAACCCCAACCTCAGTC | | |
| | | TGAGGATCTTCACCGCGCGCCTCTACTTCTGTGAG | | |
| | | GACCGCAAGGCTGAGCCCGAGGGGCTGCGGCGGC | | |
| | | TGCACCGCGCCGGGGTGCAAATAGCCATCATGACC | | |
| | | TTCAAAGATTATTTTTACTGCTGGAATACTTTTGTA | | |
| | | GAAAACCATGAAAGAACTTTCAAAGCCTGGGAAG | | |
| | | GGCTGCATGAAAATTCAGTTCGTCTCTCCAGACAG | | |
| | | CTTCGGCGCATCCTTTTGCCCCTGTATGAGGTTGAT | | |
| | | GACTTACGAGACGCATTTCGTACTTTGGGACTTTG | | |
| | | A | | |
| galK_{OFF}_gRNA | gRNA | TGAGCGGCAATCGATTCATT | 23 | This |
| | protospacer | | | work |
| recJ_gRNA | gRNA | TCACGCGAATTATTTACCGC | 24 | This |
| | protospacer | | | work |
| xonA_gRNA | gRNA | GCTTACCGTCATTCATCATT | 25 | This |
| | protospacer | | | work |
| xonA_gRNA | gRNA | GGCGATCTAACGCG | 31 | This |
| (14 bps) | protospacer | | | work |
| | (used in the | | | |
| | χHiSCRIBE | | | |
| | cassette) | | | |
| ung_gRNA | gRNA | GGACTGCCGCTCGCTGGCGA | 32 | This |
| | protospacer | | | work |

TABLE 5
List of the sequencing primers
used in this study

| Primer | | | SEQ |
|---|---|---|---|
| code | Name | Sequence | ID NO |
| FF_oligo_1831 | lacZ(+) | ACACGACGCTCTTCCGAT | 33 |
| | | CTNNNNNCTG GAA AGC | |
| | | GGG CAG TGA GC | |
| FF_oligo_1833 | lacZ(-) | CGGCATTCCTGCTGAACC | 34 |
| | | GCTCTTCCGATCTNNNNN | |
| | | CCCAGTCACGACGTTGTA | |
| | | AAACGAC | |
| FF_oligo_1890 | galK(+) | ACACGACGCTCTTCCGAT | 35 |
| | | CTNNNNNGTTTGTAGGCT | |
| | | GTAACTGCGGGATCATGG | |
| FF_oligo_1891 | galK(-) | CGGCATTCCTGCTGAACC | 36 |
| | | GCTCTTCCGATCTNNNNN | |
| | | TCACGCAGGGCTGGCTGC | |
| | | TG | |
| FF_oligo_2444 | galK_1n(+) | ACACGACGCTCTTCCGAT | 37 |
| | | CTNNNNNGCTCGGCAAGA | |
| | | AAGATCATGCCa | |
| FF_oligo_2445 | galK-1n(-) | CGGCATTCCTGCTGAACC | 38 |
| | | GCTCTTCCGATCTNNNNN | |
| | | CTGCTGGAAGAAACGCGC | |
| | | Ag | |

### Cells and Antibiotics.

Chemically competent E. coli DH5α F′ lac^{q} (NEB) was used for cloning. Unless otherwise noted, antibiotics were used at the following concentrations: carbenicillin (Carb, 50 μg/ml), kanamycin (Kan, 20 μg/ml), chloramphenicol (Cm, 30 μg/ml), streptomycin (St, 50 μg/ml), spectinomycin (Sp, 100 μg/ml), rifampicin (Rif, 100 μg/ml), and nalidixic acid (Nal, 30 μg/ml).

### Induction of Cells and Plating Assays.

KanR reversion assay was performed as described previously (13). Briefly, for each experiment, single colony transformants were separately inoculated in LB+appropriate antibiotics and grown overnight (37° C., 300 RPM) to obtain seed cultures. Unless otherwise noted, inductions were performed by diluting the seed cultures (1:1000) in LB+antibiotics±inducers followed by 24 hours incubation (37° C., 700 RPM) in 96-well plates. Aliquots of the samples were then serially diluted and spotted on selective media to determine the number of recombinant and viable cells in each culture. The number of viable cells was determined by plating aliquots of cultures on LB plates containing antibiotic marker present on the SCRIBE plasmid (Carb or Cm). LB+Kan plates were used to determine the number of recombinants. For each sample, the recombinant frequency was reported as the mean of the ratio of recombinants to viable cells for three independent replicates.

In the galK reversion assay, SCRIBE plasmids were delivered to galK_{OFF} reporter cells (with either chemical transformation, transduction or conjugation), cells were outgrown in LB for one hour without selection and plated on MacConkey+Gal+appropriate antibiotic. The ratio of pink colonies (galK_{ON}) to transformants was used as a measure of recombinant frequency. For each sample, the recombinant frequency was reported as the mean of the ratio of recombinants to viable cells for three independent replicates.

### Phagemid Packaging and Transduction.

SCRIBE phagemids were packaged into M13 phage particles as described previously (26). Briefly, SCRIBE plasmids harboring M13 origin of replication were transformed into M13 packaging strain (DH5α F⁺ PRO harboring m13cp helper plasmid (26)). Single colony transformants were grown overnight in 2 ml LB+antibiotics. The cultures were then diluted (1:100) in 50 ml fresh media and grown up to saturation. Phage particles were purified from the cultures supernatant by PEG/NaCl precipitation (38) and stored in 4° C. in SM buffer (50 mM Tris-HCl [pH 7.5]), 100 mM NaCl, 10 mM MgSO₄) for later use.

For transduction experiments, overnight cultures of the reporter strains harboring F plasmid were diluted (1:1000) in fresh media and transduced by adding purified phage particles encoding SCRIBE (MOI=50). After 1 hour incubation (37° C., 700 RPM), dilutions of the cultures were spotted on MacConkey+Gal plates and recombinant frequency was calculated as described above (galK reversion assay).

### Construct Delivery by Conjugation.

SCRIBE plasmids harboring RP4 origin of transfer were transformed into MFDpir strain (39) to produce donor strains. A spontaneous streptomycin-resistant mutant of the galK_{OFF} reporter strain was used as the recipient strain. Donor and recipient strains were grown overnight in LB with appropriate antibiotics (media for the donor strains were supplemented with 0.3 mM diaminopimelic acid (DAP) throughout the experiment). Overnight cultures of donor and recipient strains were diluted (1:100) in fresh media and grown to an OD₆₀₀˜1. Cells were pelleted and resuspended in LB, and mating pairs were mixed at a donor to recipient ratio of 1000:1 and potted onto nitrocellulose filters placed on LB agar supplemented with 0.3 mM DAP. The plates were incubated at 37° C. for 6 h to allow conjugation. Conjugation mixtures were then collected by vigorously vortexing the filters in 1 ml PBS, serially diluted and spotted on MacConkey+Gal+antibiotics plates as described in the galK reversion assay. The ratio of pink colonies per transconjugants was used as a measure of recombinant frequency.

In experiments shown in FIGS. 2C and 2D, an overnight culture of an unidentified bacterial community obtained from mouse stool was mixed (1:1) with an spontaneous streptomycin-resistant mutant of the galK_{OFF} reporter strain to build a synthetic bacterial community. This synthetic community was used as the recipient culture in the experiments shown in FIGS. 2C and 2D. The transduction and conjugation protocols were performed as described above.

### High-Throughput Sequencing.

The allele frequencies of the SCRIBE target sites (galK locus in FIGS. 1E, and 2B) were analyzed using Illumina Mi-Seq. To analyze the dynamic of enrichment of galK_{ON} allele at presence and absence of counterselection by CRISPR nuclease, SCRIBE(galK_{ON}) plasmid was transformed into galK_{OFF} reporter strain harboring either aTc-inducible Cas9 or dCas9 plasmids (shown in FIG. 1E) and transformants were selected on LB+Carb+Cm plates. After 24 hours of incubation at 37° C. (corresponding to log 2(3*10⁹)≈31 generations of growth (40)), single-colonies from transformation plate were resuspended in LB+Carb+Cm, diluted (1:1000) in fresh media and grown (37° C., 700 RPM) up to saturation (corresponding to log 2(1000)≈10 generations of growth) at presence or absence of aTc (200 ng/ml). The galK locus was amplified and 1 ul of the liquid culture (or resuspended colony) as templates. Barcodes and Illumina adopters were then added using an additional round of PCR. Samples were then gel-purified, multiplexed, and sequenced by Illumina Mi-Seq. The obtained reads were then demultiplexed into individual samples based on the attached barcodes and mapped to the reference sequence. Any reads that lacked the expected “ATGCCXXXXXXATCGAT” (SEQ ID NO: 26) motif, where “XXXXXX” corresponds to the 6 base-pair variable site (bolded) in the galK alleles (ATGCCCTCTTGATCGAT (SEQ ID NO: 41) for galK_{ON} or ATGCCTAATGAATCGAT (SEQ ID NO: 42) for galK_{OFF}), or contained ambiguous nucleotides within this region were discarded. Editing efficiency was reported as the ratio of galK_{ON} reads to the total number of galK_{ON}+galK_{OFF} reads. For galKWT to galKSYN experiment, editing efficiency was reported as the ratio of galKSYN reads to the total number of galKSYN+galKWT reads. For galK reversion experiments, editing efficiency was calculated as the ratio of galKON reads to the total number of galKON+galKOFF reads The enrichment of recombinant alleles in the WT E. coli MG1655 background (FIG. 2I) was investigated similarly. Single colonies of transformants were picked 24 h (or 48 h) after transformation, resuspended in water, and used as templates for PCR. The samples were processed as described above.

The enrichment of recombinant alleles in the WT background (FIG. 2B) was investigated similarly. 24 hours after transformation, single colonies of transformants were picked and resuspended in water and used as template for PCR, and the samples were processed as described above.

Similar strategy was used to analyze the dynamics of P_{lac} locus in the experiment shown in FIG. 3. The P_{lac} locus was amplified using primers XX and 1 ul of the liquid cultures obtained from samples at different time points throughout the experiment. Barcodes and Illumina adopters were then added using an additional round of PCR. Samples were then gel-purified, multiplexed, and sequenced by paired-end Illumina Mi-Seq for increased accuracy. Any reads that lacked the expected “YYYYYYCTTTATGCTTCCGGCTCGZZZZZZ” (SEQ ID NO: 27) motif, where “YYYYYY” and “ZZZZZZ” correspond to positions of the −35 and −10 boxes of the P_{lac} promoter respectively, or contained ambiguous nucleotides within this region were discarded. The variant frequencies were calculated as the ratio of the number of reads for a given variant to the total number of reads for that sample.

For the bacterial spatial organization recording and connectome mapping experiments (shown in FIG. 11D and FIG. 11A, respectively), barcoded donor and recipient populations were conjugated as described above. For the former experiment, conjugation mixtures were resuspended in LB and the memory registers in the galK locus were amplified by allele-specific PCR to deplete unedited registers. As shown in FIG. 11F, primers that specifically bind to the writing control nucleotide but have a mismatched nucleotide at the 3′-end position with the unedited registers were designed. These primers were then used with HiDi DNA polymerase (a selective variant of DNA polymerase that can only amplify templates that are perfectly matched at the 3′-end with a given primer, myPLOS Biotec, DE) to specifically amplify edited registers from 1 μL of conjugation mixtures while depleting the unedited registers. Illumina barcodes and adapters were then added to the samples by a second round of PCR. Samples were gel-purified, multiplexed, and sequenced by Illumina MiSeq. Samples were then computationally demultiplexed, and any reads that contained non-edited registers, which lacked any of the two expected motifs flanking the two memory registers (ATGCCTMMMMMMTCGATT (SEQ ID NO: 39) and AGTGCGNNNNNNGTGCGC (SEQ ID NO: 40), where “MMMMMM” and “NNNNNN” correspond to positions of the memory Registers 1 and 2, respectively), or that contained ambiguous nucleotides within this region were discarded. The frequencies of variants that were observed simultaneously in a single read in the two registers were then calculated and presented as weighted connectivity matrices (FIG. 11E and FIG. 11G).

### Continuous Evolution of the P_{lac} Promoter.

The efficient genomic editing achieved by SCRIBE can be coupled to a continuous selection/screening setup to allow continuous evolution of a desired target loci. In order to demonstrate this, the P_{lac} or E. coli was evolved. To achieve a wider dynamic range of evolution, a weaken P_{lac} promoter was used. This was achieve by mutating the −10 sequence of P_{lac} promoter from “TATGTT” to “CCCCCC”. This mutation leads to poor growth of cells in M9 media at presence of lactose as the sole carbon source. An overnight culture of the parental strain harboring the mutated P_{lac} promoter (MG1655 ΔrecJ ΔxonA F⁺ P_{lac(TATGTT“→”CCCCCC)}) was diluted (1:100) into M9+Glu (0.2%). The culture was divided into two sets (with three samples in each set). On set of samples received SCRIBE(P_{lac}) phagemid library and the other set received SCRIBE(NS) phagemid (MOI=100), incubated in a 96-well plate inside plate reader at 37° C. with shaking (300 RPM). After one hour incubation, Carbenicilin was added to the cultures to select for phagemid delivery. Cells were incubated in the plate reader for additional 23 hours. Samples were diluted (1:100) in 200 μl fresh M9+Lac (0.2%) media containing same phagemid composition, and the cultures were incubated for 48 hours as before. After this initial incubation, the samples were diluted (1:100) and regrown (24 hours) in M9+Lac (0.2%) containing the same composition of phagemid for 5 additional cycles. OD600 was monitored and samples were taken for Illumina sequencing throughout the experiment.

To verify the activity of the identified variants in the P_{lac} evolution experiments, these variants were reconstructed in the parental background using oligo-mediated recombineering (41). The reconstructed variants were grown overnight in LB, diluted (1:100) in fresh media supplemented with IPTG (1 mM) and grown for 8 hours at 37° C. The activity of reconstructed P_{lac} promoter variants were measured by Miller assay using Fluorescein di-β-D-galactopyranoside (FDG) as substrate. 50 μl of each culture was mixed with 50 μl of B-PER II reagent (Pierce Biotechnology) and FDG (0.005 mg/ml final concentration). The fluorescence signal (absorption/emission: 485/515) was monitored in a plate reader with continuous shaking for 2 hours. β-galactosidase activity was calculated by normalizing the rate of FDG hydrolysis (obtained from fluorescence signal) to the initial OD. For each sample, β-galactosidase activity was reported as the mean of three independent biological replicates.

### SCRIBE(P_{lac}) Phagemid Library Construction.

SCRIBE(P_{lac}) randomized phagemid library was constructed by a modified Quik-Change protocol. Briefly, a SCRIBE phagemid was PCR amplified that contain the randomized regions corresponding to −35 and −10 regions of P_{lac}. The primers also contain compatible sites for type IIS enzyme Esp3I. The PCR product was then used in a Golden-gate assembly to circularize the linear vector. The circularized vector library was then amplified by transformation into Electro-ten Blue electrocompetent cells. The amplified library then was then packaged into phagemid particles as described above.

### Calculating Mutation Rate.

Different SCRIBE plasmids (as shown in FIG. 4) were transformed into MG1655 ΔrecJ ΔxonA strain. Six single colonies from each transformation plates were inoculated in 1 mL LB+Kan media in 24-well plates and incubated (37° C., 700 RPM) for 24 hours. The number of Rif^{R} and Nal^{R} mutants in each sample were determined by plating 400 μl of each sample on LB+Rif and LB+Nal plates. The experiment was repeated 4 times (total 24 parallel culture for each sample). The mutation rate was calculated using Maximum Likelihood Estimator (MSS-MLE) method (42) using FALCOR (43).

To investigate the nature and spectrum of Rif^{R} mutations, the rpoB locus from 50 Rif^{R} colonies from each sample were PCR amplified using primers XX and after column purification were analyzed by Sanger sequencing. More than 98% of the samples contained mutations within the sequenced region.

### REFERENCES

1. N. Costantino, D. L. Court, Enhanced levels of lambda Red-mediated recombinants in mismatch repair mutants. Proceedings of the National Academy of Sciences of the United States of America 100, 15748-15753 (2003); published online EpubDec 23 (10.1073/pnas.2434959100).
2. K. A. Datsenko, B. L. Wanner, One-step inactivation of chromosomal genes in Escherichia coli K-12 using PCR products. Proc Natl Acad Sci USA 97, 6640-6645 (2000); published online EpubJun 6 (10.1073/pnas.120163297 [pii]).
3. G. Pines, E. F. Freed, J. D. Winkler, R. T. Gill, Bacterial Recombineering: Genome Engineering via Phage-Based Homologous Recombination. ACS synthetic biology 4, 1176-1185 (2015); published online EpubNov 20 (10.1021/acssynbio.5b00009).
4. B. Swingle, E. Markel, N. Costantino, M. G. Bubunenko, S. Cartinhour, D. L. Court, Oligonucleotide recombination in Gram-negative bacteria. Molecular microbiology 75, 138-148 (2010); published online EpubJan (10.1111/j.1365-2958.2009.06976.x).
5. D. Yu, H. M. Ellis, E. C. Lee, N. A. Jenkins, N. G. Copeland, D. L. Court, An efficient recombination system for chromosome engineering in Escherichia coli. Proceedings of the National Academy of Sciences of the United States of America 97, 5978-5983 (2000); published online EpubMay 23 (10.1073/pnas.100127597).
6. H. H. Wang, F. J. Isaacs, P. A. Carr, Z. Z. Sun, G. Xu, C. R. Forest, G. M. Church, Programming cells by multiplex genome engineering and accelerated evolution. Nature 460, 894-898 (2009); published online EpubAug 13 (10.1038/nature08187).
7. J. W. Drake, A constant rate of spontaneous mutation in DNA-based microbes. Proceedings of the National Academy of Sciences of the United States of America 88, 7160-7164 (1991).
8. M. Lynch, Evolution of the mutation rate. Trends in genetics: TIG 26, 345-352 (2010); published online EpubAug (10.1016/j.tig.2010.05.003).
9. H. Guo, D. Arambula, P. Ghosh, J. F. Miller, Diversity-generating Retroelements in Phage and Bacterial Genomes. Microbiology spectrum 2, (2014); published online EpubDec (10.1128/microbiolspec.MDNA3-0029-2014).
10. K. W. Deitsch, S. A. Lukehart, J. R. Stringer, Common strategies for antigenic variation by bacterial, fungal and protozoan pathogens. Nature reviews. Microbiology 7, 493-503 (2009); published online EpubJul (10.1038/nrmicro2145).
11. G. H. Palmer, T. Bankhead, H. S. Seifert, Antigenic Variation in Bacterial Pathogens. Microbiology spectrum 4, (2016); published online EpubFeb (10.1128/microbiolspec.VMBF-0005-2015).
12. L. Salaun, L. A. Snyder, N. J. Saunders, Adaptation by phase variation in pathogenic bacteria. Advances in applied microbiology 52, 263-301 (2003).
13. F. Farzadfard, T. K. Lu, Synthetic biology. Genomically encoded analog memory with precise in vivo DNA writing in living cell populations. Science 346, 1256272 (2014); published online EpubNov 14 (10.1126/science. 1256272).
14. J. A. Sawitzke, N. Costantino, X. T. Li, L. C. Thomason, M. Bubunenko, C. Court, D. L. Court, Probing cellular processes with oligo-mediated recombination and using the knowledge gained to optimize recombineering. Journal of molecular biology 407, 45-59 (2011); published online EpubMar 18 (10.1016/j.jmb.2011.01.030).
15. W. K. Maas, C. Wang, T. Lima, A. Hach, D. Lim, Multicopy single-stranded DNA of Escherichia coli enhances mutation and recombination frequencies by titrating MutS protein. Molecular microbiology 19, 505-509 (1996).
16. B. E. Dutra, V. A. Sutera, Jr., S. T. Lovett, RecA-independent recombination is efficient but limited by exonucleases. Proceedings of the National Academy of Sciences of the United States of America 104, 216-221 (2007); published online EpubJan 2 (10.1073/pnas.0608293104).
17. K. C. Murphy, M. G. Marinus, RecA-independent single-stranded DNA oligonucleotide-mediated mutagenesis. F1000 biology reports 2, 56 (2010); published online EpubJul 22 (10.3410/B2-56).
18. J. W. Chase, C. C. Richardson, Exonuclease VII of Escherichia coli. Mechanism of action. The Journal of biological chemistry 249, 4553-4561 (1974).
19. J. A. Mosberg, C. J. Gregg, M. J. Lajoie, H. H. Wang, G. M. Church, Improving lambda red genome engineering in Escherichia coli via rational removal of endogenous nucleases. PloS one 7, e44638 (2012) 10.1371/journal.pone.0044638).
20. H. Jung, J. Liang, Y. Jung, D. Lim, Characterization of cell death in Escherichia coli mediated by XseA, a large subunit of exonuclease VII. Journal of microbiology 53, 820-828 (2015); published online EpubDec (10.1007/s 12275-015-5304-0).
21. M. S. Dillingham, S. C. Kowalczykowski, RecBCD enzyme and the repair of double-stranded DNA breaks. Microbiology and molecular biology reviews: MMBR 72, 642-671, Table of Contents (2008); published online EpubDec (10.1128/MMBR.00020-08).
22. L. S. Qi, M. H. Larson, L. A. Gilbert, J. A. Doudna, J. S. Weissman, A. P. Arkin, W. A. Lim, Repurposing CRISPR as an RNA-guided platform for sequence-specific control of gene expression. Cell 152, 1173-1183 (2013); published online EpubFeb 28 (10.1016/j.cell.2013.02.022).
23. A. Novick, M. Weiner, Enzyme Induction as an All-or-None Phenomenon. Proceedings of the National Academy of Sciences of the United States of America 43, 553-566 (1957).
24. M. S. Huen, X. T. Li, L. Y. Lu, R. M. Watt, D. P. Liu, J. D. Huang, The involvement of replication in single stranded oligonucleotide-mediated gene repair. Nucleic acids research 34, 6183-6194 (2006) 10.1093/nar/gk1852).
25. R. M. Schaaper, R. L. Dunn, Spectra of spontaneous mutations in Escherichia coli strains defective in mismatch correction: the nature of in vivo DNA replication errors. Proceedings of the National Academy of Sciences of the United States of America 84, 6220-6224 (1987).
26. L. Chasteen, J. Ayriss, P. Pavlik, A. R. Bradbury, Eliminating helper phage from phage display. Nucleic acids research 34, e145 (2006) 10.1093/nar/gk1772).
27. R. J. Citorik, M. Mimee, T. K. Lu, Sequence-specific antimicrobials using efficiently delivered RNA-guided nucleases. Nature biotechnology 32, 1141-1145 (2014); published online EpubNov (10.1038/nbt.3011).
28. M. G. Ross, C. Russ, M. Costello, A. Hollinger, N. J. Lennon, R. Hegarty, C. Nusbaum, D. B. Jaffe, Characterizing and measuring bias in sequence data. Genome biology 14, R51 (2013) 10.1186/gb-2013-14-5-r51).
29. J. K. Rogers, N. D. Taylor, G. M. Church, Biosensor-based engineering of biosynthetic pathways. Current opinion in biotechnology 42, 84-91 (2016); published online EpubMar 18 (10.1016/j.copbio.2016.03.005).
30. D. J. Jin, C. A. Gross, Mapping and sequencing of mutations in the Escherichia coli rpoB gene that lead to rifampicin resistance. Journal of molecular biology 202, 45-58 (1988).
31. Y. A. Ovchinnikov, G. S. Monastyrskaya, S. O. Guriev, N. F. Kalinina, E. D. Sverdlov, A. I. Gragerov, I. A. Bass, I. F. Kiver, E. P. Moiseyeva, V. N. Igumnov, S. Z. Mindlin, V. G. Nikiforov, R. B. Khesin, RNA polymerase rifampicin resistance mutations in Escherichia coli: sequence changes and dominance. Molecular & general genetics: MGG 190, 344-348 (1983).
32. J. Hrebenda, H. Heleszko, K. Brzostek, J. Bielecki, Mutation affecting resistance of Escherichia coli K12 to nalidixic acid. Journal of general microbiology 131, 2285-2292 (1985); published online EpubSep (10.1099/00221287-131-9-2285).
33. S. K. Petersen-Mahrt, R. S. Harris, M. S. Neuberger, AID mutates E. coli suggesting a DNA deamination mechanism for antibody diversification. Nature 418, 99-103 (2002); published online EpubJul 4 (10.1038/nature00862).
34. A. S. Bhagwat, W. Hao, J. P. Townes, H. Lee, H. Tang, P. L. Foster, Strand-biased cytosine deamination at the replication fork causes cytosine to thymine mutations in Escherichia coli. Proceedings of the National Academy of Sciences of the United States of America 113, 2176-2181 (2016); published online EpubFeb 23 (10.1073/pnas.1522325113).
35. S. Brakmann, S. Grzeszik, An error-prone T7 RNA polymerase mutant generated by directed evolution. Chembiochem: a European journal of chemical biology 2, 212-219 (2001).
36. K. Bebenek, J. Abbotts, S. H. Wilson, T. A. Kunkel, Error-prone polymerization by HIV-1 reverse transcriptase. Contribution of template-primer misalignment, miscoding, and termination probability to mutational hot spots. The Journal of biological chemistry 268, 10324-10334 (1993).
37. B. Medhekar, J. F. Miller, Diversity-generating retroelements. Current opinion in microbiology 10, 388-395 (2007); published online EpubAug (10.1016/j.mib.2007.06.004).
38. K. R. Yamamoto, B. M. Alberts, R. Benzinger, L. Lawhorne, G. Treiber, Rapid bacteriophage sedimentation in the presence of polyethylene glycol and its application to large-scale virus purification. Virology 40, 734-744 (1970).
39. L. Ferrieres, G. Hemery, T. Nham, A. M. Guerout, D. Mazel, C. Beloin, J. M. Ghigo, Silent mischief: bacteriophage Mu insertions contaminate products of Escherichia coli random mutagenesis performed using suicidal transposon delivery plasmids mobilized by broad-host-range RP4 conjugative machinery. Journal of bacteriology 192, 6418-6427 (2010); published online EpubDec (10.1128/JB.00621-10).
40. R. Milo, P. Jorgensen, U. Moran, G. Weber, M. Springer, BioNumbers—the database of key numbers in molecular and cell biology. Nucleic acids research 38, D750-753 (2010); published online EpubJan (10.1093/nar/gkp889).
41. W. Chan, N. Costantino, R. Li, S. C. Lee, Q. Su, D. Melvin, D. L. Court, P. Liu, A recombineering based approach for high-throughput conditional knockout targeting vector construction. Nucleic acids research 35, e64 (2007) 10.1093/nar/gkm163).
42. S. Sarkar, W. T. Ma, G. H. Sandri, On fluctuation analysis: a new, simple and efficient method for computing the expected number of mutants. Genetica 85, 173-179 (1992).
43. B. M. Hall, C. X. Ma, P. Liang, K. K. Singh, Fluctuation analysis CalculatOR: a web tool for the determination of mutation rate using Luria-Delbruck fluctuation analysis. Bioinformatics 25, 1564-1565 (2009); published online EpubJun 15 (10.1093/bioinformatics/btp253).
44. R. Lutz, H. Bujard, Independent and tight regulation of transcriptional units in Escherichia coli via the LacR/O, the TetR/O and AraC/I1-I2 regulatory elements. Nucleic Acids Res 25, 1203-1210 (1997); published online EpubMar 15 (gka167 [pii]).
45. L. Zelcbuch, N. Antonovsky, A. Bar-Even, A. Levin-Karp, U. Barenholz, M. Dayagi, W. Liebermeister, A. Flamholz, E. Noor, S. Amram, A. Brandis, T. Bareia, I. Yofe, H. Jubran, R. Milo, Spanning high-dimensional expression space using ribosome-binding site combinatorics. Nucleic acids research 41, e98 (2013); published online EpubMay 50 (10.1093/nar/gkt151).
46. W. Jiang, D. Bikard, D. Cox, F. Zhang, L. A. Marraffini, RNA-guided editing of bacterial genomes using CRISPR-Cas systems. Nature biotechnology 31, 233-239 (2013); published online EpubMar (10.1038/nbt.2508).
47. C. Ronda, L. E. Pedersen, M. O. Sommer, A. T. Nielsen, CRMAGE: CRISPR Optimized MAGE Recombineering. Scientific reports 6, 19452 (2016); published online EpubJan 22 (10.1038/srep 19452).
48. L. Cui, D. Bikard, Consequences of Cas9 cleavage in the chromosome of Escherichia coli. Nucleic acids research 44, 4243-4251 (2016); published online EpubMay 19 (10.1093/nar/gkw223).
49. B. J. Caliando, C. A. Voigt, Targeted DNA degradation using a CRISPR device stably carried in the host genome. Nature communications 6, 6989 (2015); published online EpubMay 19 (10.1038/ncomms7989).
50. Y. Gao, Y. Zhao, Self-processing of ribozyme-flanked RNAs into guide RNAs in vitro and in vivo for CRISPR-mediated genome editing. Journal of integrative plant biology 56, 343-349 (2014); published online EpubApr (10.1111/jipb.12152).
51. D. I. Lou, J. A. Hussmann, R. M. McBee, A. Acevedo, R. Andino, W. H. Press, S. L. Sawyer, High-throughput DNA sequencing errors are reduced by orders of magnitude using circle sequencing. Proceedings of the National Academy of Sciences of the United States of America 110, 19872-19877 (2013); published online EpubDec 3 (10.1073/pnas.1319590110).
52. M. W. Schmitt, S. R. Kennedy, J. J. Salk, E. J. Fox, J. B. Hiatt, L. A. Loeb, Detection of ultra-rare mutations by next-generation sequencing. Proceedings of the National Academy of Sciences of the United States of America 109, 14508-14513 (2012); published online EpubSep 4 (10.1073/pnas.1208715109).
53. M. Kirschner, J. Gerhart, Evolvability. Proceedings of the National Academy of Sciences of the United States of America 95, 8420-8427 (1998); published online EpubJul 21
54. A. Mayer, T. Mora, O. Rivoire, A. M. Walczak, Diversity of immune strategies explained by adaptation to pathogen statistics. Proceedings of the National Academy of Sciences of the United States of America 113, 8630-8635 (2016); published online EpubAug 2 (10.1073/pnas.1600663113).
55. J. M. Di Noia, M. S. Neuberger, Molecular mechanisms of antibody somatic hypermutation. Annual review of biochemistry 76, 1-22 (2007) 10.1146/annurev.biochem.76.061705.090740).
56. P. Horvath, R. Barrangou, CRISPR/Cas, the immune system of bacteria and archaea. Science 327, 167-170 (2010); published online EpubJan 8 (10.1126/science.1179555).
57. R. Sorek, C. M. Lawrence, B. Wiedenheft, CRISPR-mediated adaptive immune systems in bacteria and archaea. Annual review of biochemistry 82, 237-266 (2013) 10.1146/annurev-biochem-072911-172315).
58. S. H. Sternberg, H. Richter, E. Charpentier, U. Qimron, Adaptation in CRISPR-Cas Systems. Molecular cell 61, 797-808 (2016); published online EpubMar 17 (10.1016/j.molcel.2016.01.030). microbiology 10, 388-395 (2007); published online EpubAug (10.1016/j.mib.2007.06.004).
59. K. Nishikura, Functions and regulation of RNA editing by ADAR deaminases. Annual review of biochemistry 79, 321-349 (2010) 10.1146/annurev-biochem-060208-105251).
60. N. Roquet, A. P. Soleimany, A. C. Ferris, S. Aaronson, T. K. Lu, Synthetic recombinase-based state machines in living cells. Science 353, aad8559 (2016); published online EpubJul 22 (10.1126/science.aad8559).
61. S. L. Shipman, J. Nivala, J. D. Macklis, G. M. Church, Molecular recordings by directed CRISPR spacer acquisition. Science 353, aaf1175 (2016); published online EpubJul 29 (10.1126/science.aaf1175).
62. S. D. Perli, C. H. Cui, T. K. Lu, Continuous genetic recording with self-targeting CRISPR-Cas in human cells. Science 353, (2016); published online EpubSep 09 (10.1126/science.aag0511).
63. R. I. Zeitoun, A. D. Garst, G. D. Degen, G. Pines, T. J. Mansell, T. Y. Glebes, N. R. Boyle, R. T. Gill, Multiplexed tracking of combinatorial genomic mutations in engineered cell populations. Nature biotechnology 33, 631-637 (2015); published online EpubJun (10.1038/nbt.3177).
64. T. Aparicio, S. I. Jensen, A. T. Nielsen, V. de Lorenzo, E. Martinez-Garcia, The Ssr protein (T1E_1405) from Pseudomonas putida DOT-T1E enables oligonucleotide-based recombineering in platform strain P. putida EM42. Biotechnology journal 11, 1309-1319 (2016); published online EpubOct (10.1002/biot.201600317).
65. C. D. Nadell, K. Drescher, K. R. Foster, Spatial structure, cooperation and competition in biofilms. Nature reviews. Microbiology 14, 589-600 (2016); published online EpubSep (10.1038/nrmicro.2016.84).
66. A. M. Zador, J. Dubnau, H. K. Oyibo, H. Zhan, G. Cao, I. D. Peikon, Sequencing the connectome. PLoS biology 10, e1001411 (2012) 10.1371/journal.pbio.1001411).
67. J. I. Glaser, B. M. Zamft, G. M. Church, K. P. Kording, Puzzle Imaging: Using Large-Scale Dimensionality Reduction Algorithms for Localization. PloS one 10, e0131593 (2015) 10.1371/journal.pone.0131593).
68. I. D. Peikon, J. M. Kebschull, V. V. Vagin, D. I. Ravens, Y. C. Sun, E. Brouzes, I. R. Correa, Jr., D. Bressan, A. M. Zador, Using high-throughput barcode sequencing to efficiently map connectomes. Nucleic acids research, (2017); published online EpubApr 26 (10.1093/nar/gkx292).
69. S. L. Shipman, J. Nivala, J. D. Macklis, G. M. Church, CRISPR-Cas encoding of a digital movie into the genomes of a population of living bacteria. Nature 547, 345-349 (2017); published online EpubJul 20 (10.1038/nature23017).
70. V. A. Risso, J. A. Gavira, D. F. Mejia-Carmona, E. A. Gaucher, J. M. Sanchez-Ruiz, Hyperstability and substrate promiscuity in laboratory resurrections of Precambrian beta-lactamases. Journal of the American Chemical Society 135, 2899-2902 (2013); published online EpubFeb 27 (10.1021/ja311630a).
71. J. W. Thornton, Resurrecting ancient genes: experimental analysis of extinct molecules. Nature reviews. Genetics 5, 366-375 (2004); published online EpubMay (10.1038/nrg1324).
72. T. M. Jermann, J. G. Opitz, J. Stackhouse, S. A. Benner, Reconstructing the evolutionary history of the artiodactyl ribonuclease superfamily. Nature 374, 57-59 (1995); published online EpubMar 2 (10.1038/374057a0).
73. D. M. Weinreich, N. F. Delaney, M. A. Depristo, D. L. Hartl, Darwinian evolution can follow only very few mutational paths to fitter proteins. Science 312, 111-114 (2006); published online EpubApr 7 (10.1126/science. 1123539).
74. C. Pal, B. Papp, G. Posfai, The dawn of evolutionary genome engineering. Nature reviews. Genetics 15, 504-512 (2014); published online EpubJul (10.1038/nrg3746).
75. D. G. Gibson, Enzymatic assembly of overlapping DNA fragments. Methods in enzymology 498, 349-361 (2011) 10.1016/B978-0-12-385120-8.00015-2).
76. C. Engler, S. Marillonnet, Golden Gate cloning. Methods in molecular biology 1116, 119-131 (2014) 10.1007/978-1-62703-764-8_9).
77. B. G. Hall, H. Acar, A. Nandipati, M. Barlow, Growth rates made easy. Molecular biology and evolution 31, 232-238 (2014); published online EpubJan (10.1093/molbev/mst187).
78. A. C. Komor, Y. B. Kim, M. S. Packer, J. A. Zuris, D. R. Liu, Programmable editing of a target base in genomic DNA without double-stranded DNA cleavage. Nature 533, 420-424 (2016); published online EpubMay 19 (10.1038/nature17946).
79. P. Siuti, J. Yazbek, T. K. Lu, Synthetic circuits integrating logic and memory in living cells. Nature biotechnology 31, 448-452 (2013); published online EpubMay

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An engineered nucleic acid construct comprising:
(a) a nucleotide sequence encoding a guide RNA targeting an exonuclease;
(b) a nucleotide sequence encoding a single-stranded msrRNA and a single-stranded msdDNA modified to contain a targeting sequence, wherein (b) is flanked by a pair of inverted repeat sequences; and
(c) a nucleotide sequence encoding a reverse transcriptase protein.

2. The engineered nucleic acid construct of claim 1, wherein the nucleotide sequence of (a) further encodes at least one other guide RNA targeting at least one other exonuclease and/or at least one ribozyme downstream from a guide RNA of (a).

3. (canceled)

4. The engineered nucleic acid construct of claim 2, wherein the at least one ribozyme is selected from a Hepatitis delta virus ribozyme (HDVR) and a hammerhead ribozyme (HHR).

5. The engineered nucleic acid construct of claim 1, wherein an exonuclease of (a) is selected from RecJ, XonA and ExoX.

6. The engineered nucleic acid construct of claim 5, wherein a guide RNA of (a) targets RecJ and at least one other guide RNA of (a) targets XonA, and optionally wherein at least one other guide RNA of (a) targets ExoX.

7. (canceled)

8. The engineered nucleic acid construct of claim 1, wherein the engineered nucleic acid construct further comprises a nucleotide sequence encoding catalytically-inactive Cas9 (dCas9) and/or a nucleotide sequence encoding a single-stranded DNA (ssDNA)-annealing recombinase protein.

9. (canceled)

10. The engineered nucleic acid construct of claim 8, wherein the ssDNA-annealing recombinase protein is a bacteriophage lambda Beta recombinase protein or a bacteriophage lambda Beta recombinase protein homolog.

11. The engineered nucleic acid construct of claim 1, wherein (a) is upstream of (b), wherein (b) is upstream of (c), and/or wherein (a), (b) and (c) are operably linked to a promoter, optionally wherein the promoter is an inducible promoter.

12-14. (canceled)

15. The engineered nucleic acid construct of claim 1, wherein (a) is operably linked to a promoter, (b) is operably linked to a promoter that is different from the promoter operably linked to (a), and (c) is operably linked to a promoter that is different from the promoter operably linked to (a) and the promoter operably linked to (b).

16-19. (canceled)

20. The engineered nucleic acid construct of claim 1, wherein the targeting sequence of (b) targets an undesired allele of a gene of a bacterial cell.

21. The engineered nucleic acid construct of claim 20, wherein the gene of the bacterial cell is a wild-type gene that adversely effects cell growth and/or viability under a stress condition.

22. A composition, kit, or cell comprising the engineered nucleic acid construct of claim 1.

23-28. (canceled)

29. A cell, comprising:
(a) an engineered nucleic acid encoding a guide RNA targeting an exonuclease;
(b) an engineered nucleic acid encoding a single-stranded msrRNA and a single-stranded msdDNA modified to contain a targeting sequence, wherein (b) is flanked by a pair of inverted repeat sequences; and
(c) an engineered nucleic acid encoding a reverse transcriptase protein, optionally wherein the engineered nucleic acid of (b) and (c) are components of a single nucleic acid molecule.

30-35. (canceled)

36. A method comprising delivering to a cell an engineered nucleic acid construct of claim 1, wherein the cell comprises at least one target nucleotide sequence that is complementary to the targeting sequence of the single-stranded msdDNA, optionally further comprising delivering to the cell a single-stranded DNA-annealing recombinase protein and a catalytically-inactive Cas9 protein.

37-48. (canceled)

49. The method of claim 36, wherein the targeting sequence targets a gene specific to a bacterial cell subpopulation, the cell is a bacterial cell of the bacterial cell subpopulation, and delivery of the engineered nucleic acid construct results in modification of the bacterial cell subpopulation.

50-53. (canceled)

54. A method of mapping cellular interactions, comprising:
(a) delivering to a donor cell within a population of recipient cells (i) a transfer vector comprising a gene editing system that introduces a genetic d-barcode into a locus of the genome of the donor cells and is capable of introducing a d-barcode into a locus of the genome of the recipient cells or (ii) d-barcode that is introduced into a locus of the genome of the donor cells and is capable of being introduced into a locus of the genome of the recipient cells, wherein the recipient cells comprise a r-barcode that is different from the d-barcode, optionally located in a locus of the genome of the recipient cells;
(b) collecting the donor cell and at least one recipient cell; and
(c) sequencing the loci of the genome of the donor cells and the at least one recipient cell to map interactions among the donor cell and the at least one recipient cell.

55-69. (canceled)

70. A method improving fitness of bacterial cells, comprising
(a) delivering to bacterial cells an engineered nucleic acid construct comprising:
(i) a nucleotide sequence encoding a guide RNA targeting an exonuclease;
(ii) a nucleotide sequence encoding a single-stranded msrRNA and a single-stranded msdDNA modified to contain a targeting sequence that targets an allele of a bacterial cell gene that adversely effects fitness of the bacterial cell under a stress condition; and
(iii) a nucleotide sequence encoding an error-prone reverse transcriptase protein,
wherein (ii) is flanked by a pair of inverted repeat sequences;
(b) culturing bacterial cells of (a) under a stress condition; and
(c) collecting viable bacterial cells of (b).

71-74. (canceled)

75. The method of claim 36, wherein the targeting sequence targets a genomic locus in the cell; and
optionally a nucleotide sequence encoding an error-prone RNA polymerase or a reverse transcriptase protein, wherein delivery of the engineered nucleic acid construct results in diversification of the genomic locus of the cell, and optionally wherein the method further comprises delivering to the cell a nucleic acid-modifying enzyme or a nucleic acid encoding a nucleic acid-modifying enzyme, and error-prone RNA polymerase or a nucleic acid encoding error-prone RNA polymerase.

76-88. (canceled)

89. The method of claim 36, wherein the targeting sequence targets a naturally silent gene in the cell, the cell is a bacterial cell, and delivery of the engineered nucleic acid results in activation of the naturally silent gene in the cell.

90-92. (canceled)

93. A bacterial cell that displays surface antibodies, comprising an engineered nucleic acid construct comprising:
(a) a nucleotide sequence encoding a guide RNA targeting an exonuclease;
(b) a nucleotide sequence encoding a single-stranded msrRNA and a single-stranded msdDNA modified to contain a targeting sequence that targets in a bacterial cell a nucleotide sequence encoding an antibody, wherein (b) is flanked by a pair of inverted repeat sequences; and
(c) a nucleotide sequence encoding an error-prone reverse transcriptase protein.
